(19) United States
(12) Patent Application Publication
(10) Pub. No.: US 2025/0244419 A1
(43) Pub. Date: Jul. 31, 2025

(54) INFUSION DEVICE FOR THE PREPARATION AND DELIVERY OF MRI PROBES

(71) Applicant: The United States of America, as represented by the Secretary, Department of Health and Human Servic, Bethesda, MD (US)

(72) Inventors: Rolf E. Swenson, Silver Spring, MD (US); Jessica H. Ettedgui-Benjamini, North Bethesda, MD (US); Murali K. Cherukuri, Potomac, MD (US); Carolyn Woodroofe Hitko, Olney, MD (US); Natarajan Raju, Germantown, MD (US)

(73) Assignee: The United States of America, as represented by the Secretary, Department of Health and Human Servic, Bethesda, MD (US)

(21) Appl. No.: 18/854,462

(22) PCT Filed: Apr. 7, 2023
(86) PCT No.: PCT/US2023/017895

§ 371 Date: Oct. 4, 2024

Related U.S. Application Data
(60) Provisional application No. 63/328,556, filed on Apr. 7, 2022.

(51) Int. Cl. G01R 33/28; A61K 49/10; A61M 5/00

(52) U.S. Cl. CPC G01R 33/282; A61K 49/10; A61M 5/007

(57) ABSTRACT

Disclosed is a sterile MRI probe infusion device for preparing and administering a hyperpolarized MRI probe to a patient in need thereof. The device includes one or more reaction chambers, where a hyperpolarized MRI probe is prepared, separated from the reaction mixture, concentrated, and a solution of suitable concentration for administration to a patient is prepared. Also disclosed is a method of preparing and administering a hyperpolarized MRI probe by the use of the device to a patient in need thereof for diagnosing stages of a disease or an adverse condition or monitoring progress of a treatment of the patient having a disease or an adverse condition.

### CROSS-REFERENCE TO RELATED APPLICATIONS

This application claims priority to U.S. Provisional Patent Application No. 63/328,556, filed Apr. 7, 2022, which is incorporated by reference herein in its entirety.

### STATEMENT REGARDING FEDERALLY SPONSORED RESEARCH OR DEVELOPMENT

This invention was made with Government support and the Government has certain rights in this invention.

### BACKGROUND OF THE INVENTION

Nuclear magnetic resonance spectroscopy (NMR) and magnetic resonance imaging (MRI) methods are powerful tools widely used in biomedical, chemical, and materials science applications. These methods rely on the population difference of nuclear spin energy levels (called polarization) created after applying a strong magnetic field. Spins aligned with or against the applied field produce a net polarization, which is detected. Unfortunately, the nuclear polarization at thermal equilibrium (i.e., normal conditions) is inherently poor and remains a limitation to sensitivity and scope of the capabilities of magnetic resonance in general (Günther, NMR Spectrosc. Basic Princ. Concepts Appl. Chem., 13-28 (2013)).

Hyperpolarization techniques have been developed to overcome this problem and allow orders of magnitude NMR/MRI signal enhancement. The most widely used hyperpolarization techniques employ polarization transfer from electrons (dynamic nuclear polarization, DNP) (Hausser et al., Adv. Magn. Opt. Reson., 3, 79-139 (1968); Abagam et al., Reports Prog. Phys., 41, 395-467 (1978); and Ardenkjaer-Larsen et al., Proc. Natl. Acad. Sci. U.S.A., 100, 10158-10163 (2003)), photons (spin exchange optical pumping) (Bhaskar et al., Phys. Rev. Lett., 49, 25 (1982); Ebert et al., Lancet, 347, 1297 (1996); Albert et al., Nature, 370, 199-201 (1994); and Schroder et al., Science, 314, 446-449 (2006)), or parahydrogen (parahydrogen-induced polarization, PHIP) (Bowers et al., Phys. Rev. Lett., 57, 2645 (1986); Bowers et al., J. Am. Chem. Soc., 109, 5541-5542 (1987); Eisenschmid et al., J. Am. Chem. Soc., 109, 8089 (1987); Haake et al. J. Am. Chem. Soc., 118, 8688 (1996); Goldman et al., C. R. Phys., 6, 575 (2005); and Chekmenev et al., J. Am. Chem. Soc., 130, 4212 (2008)). Hyperpolarized magnetic resonance (MR) is an emerging molecular imaging method to monitor metabolism, enzymatic conversions, or biochemical pathways, previously inaccessible using MR.

Current hyperpolarized imaging with dissolution DNP and superconducting MRI scanners is very powerful because of its unique ability to track chemical transformations in vivo. However, DNP based experiments are relatively burdensome, slow, and expensive.

The PHIP approach and its subcategory SABRE (signal amplification by reversible exchange) allow the transfer of the 100% pure singlet spin order of parahydrogen (para-H₂) into a target molecule. The PHIP method is a traditional hydrogenative method and relies on a catalytic hydrogenation reaction where a precursor, in the form of a hydrogen acceptor, is reduced by the parahydrogen and polarized. In contrast, the reversible exchange using SABRE leaves the hyperpolarized agent chemically unchanged. It is also not limited to one para-H₂ molecule per molecule and therefore multiple spin transfer steps can lead to impressive levels of hyperpolarization. This effect has also been shown to transfer polarization to nuclei such as ¹H, ¹³C, ¹⁹F, ³¹P and ¹⁵N and/or ²⁹Si nuclei in a wide range of biologically relevant molecules (Barskiy et al., ChemPhysChem, 18, 1493-1498 (2017); Theis et al., J. Am. Chem. Soc., 137, 1404-1407 (2015); Shchepin et al., ChemPhysChem, 18, 1961-1965 (2017); Zhivonitko et al., Chem. Commun., 51, 2506-2509 (2015); Iali et al., Angew. Chemie-Int. Ed., 58, 10271-10275 (2019); and Gemeinhardt et al., Angew. Chemie Int. Ed., 59, 418-423 (2019)).

High polarization percentage, short signal build-up times, low cost, and scalability make SABRE a promising modality for studying metabolism in vivo using magnetic resonance spectroscopy technique.

The presently available hyperpolarized contrast agents come associated with a spin transfer catalyst component which contains a heavy metal, e.g., a transition metal atom, necessary to enable polarization transfer from para-H₂ to the substrate. Toxicity concerns are raised when the hyperpolarization contrast agents are administered in vivo due to the presence of potentially toxic heavy metal-based complexes (e.g., catalysts are typically Ir-based organometallic compounds) in solution along with hyperpolarized contrast agents.

Another obstacle to the successful implementation of the SABRE process is the lower solubility of parahydrogen (H₂ solubility in water is about 1.6 mg/L) in water compared to that in alcohol-based solvents. The SABRE hyperpolarization is mostly active in organic solvents, preferably methanol, which is not compatible with in vivo administration.

The foregoing shows that there exists a need for improved SABRE catalysts that are easily separable from a hyperpolarized substrate such that the hyperpolarized substrate is free of a heavy metal. There further exists a need for a method for separating a hyperpolarized substrate from the SABRE catalyst and/or hyperpolarized SABRE catalyst complex containing a heavy metal. There also exists a need for a method of administering a hyperpolarized substrate in a solvent medium that is suitable for in vivo administration.

Since the hyperpolarized MRI probes have very short life times, there is an unmet need for a device that can in situ generate and administer the hyperpolarized MRI probe to a patient in need thereof. Devices have been proposed in the art with a view to meet this need (Maslana et al., J Autom. Methods Manag. Chem., 22(6), 187-194 (2000); Kainz et al., “Synthesis of perfluoroalkyl-substituted aryl bromides and their purification over fluorous reverse phase silica,” Synthesis (10), 1425-1427 (1998)); however, the proposed devices may not be effective due to the disclosed method of hyperpolarization and/or the separation of the hyperpolarized probe from the reaction mixture. Previous attempts to automate and design instrumentation for generating hyperpolarized MRI probes or other molecules of interest have suffered from several limitations. Frequently the polarity of the Jr catalyst and the hyperpolarized MRI probe have similar solubility in methanol or other solvents in order to have efficient hyperpolarization. Attempts to use extraction between an aqueous layer and an organic layer do not always separate the hyperpolarized MRI probe from the catalyst, as the activated catalyst is to some extent soluble in water (Iali et al., “Achieving High Levels of NMR-Hyperpolarization in Aqueous Media with Minimal Catalyst Contamination using SABRE,” Chem.—A. Eur. J., 23, 10491-10495 (2017)). The Ir catalyst has been attached to silica gel to allow filtration, but the hyperpolarization levels have suffered. The Ir catalyst has been captured by solid supported thiols, with mixed success (Barskiy et al., “Rapid Catalyst Capture Enables Metal-Free Parahydrogen-Based Hyperpolarized Contrast Agents,” J. Phys. Chem. Lett. 9(11), 2721-2724 (2018); Kidd et al., “Facile Removal of Homogenous SABRE Catalysts for Purifying Hyperpolarized Metronidazole, a Potential Hypoxia Sensor,” J. Phys. Chem. 122, 16848-16852 (2018)). Furthermore, the previous instrumentation was lacking in clinical capabilities due to a lack of controls for purity and safety. Moreover, a device that disclosed the exhausting of used gases into a fume hood would not be practical in a clinical setting where such devices are useful.

The foregoing shows that there exists a need for improved SABRE catalysts that are easily separable from a hyperpolarized substrate such that the hyperpolarized substrate is free of a heavy metal. There further exists a need for a method for separating a hyperpolarized substrate from the SABRE catalyst and/or hyperpolarized SABRE catalyst complex containing a heavy metal. There also exists a need for a method of administering a hyperpolarized substrate in a solvent medium that is suitable for in vivo administration.

The invention provides such SABRE catalysts and methods. These and other advantages of the invention, as well as additional inventive features, will be apparent from the description of the invention provided herein.

### BRIEF SUMMARY OF THE INVENTION

The present disclosure provides an MRI probe infusion device that satisfies the unresolved needs highlighted above. In accordance with a first aspect of the present disclosure, an MRI probe infusion device is provided, comprising:
(i) one or more reaction chambers, each reaction chamber comprising:
   (a) a structure configured to attenuate a magnetic field within the reaction chamber from an external source, wherein a strength of the magnetic field within the reaction chamber from the external source is less than a threshold value;
   (b) one or more inlet ports;
   (c) a coil configured to generate an electro-magnetic field within the reaction chamber;
   (d) one or more temperature control devices; and
   (e) one or more outlet ports;
(ii) one or more MRI probe separators configured to receive a reaction mixture containing a perfluorinated SABRE catalyst, a solvent, and a hyperpolarized MRI probe from the one or more reaction chambers and extract the hyperpolarized MRI probe from the reaction mixture; and
(iii) one or more MRI probe collectors configured to form a solution containing a desired concentration of the hyperpolarized MRI probe.

In accordance with an embodiment of the first aspect, the structure is a mu-metal shield that attenuates a magnetic field from the external source to have a strength of less than or equal to 10 nT in the one or more reaction chambers.

In accordance with an embodiment of the first aspect, the one or more inlet ports include one or more gas ports and one or more liquid ports.

In accordance with an embodiment of the first aspect, each reaction chamber is configured to withstand a gas pressure of at least 10 bars.

In accordance with an embodiment of the first aspect, the magnetic field within the reaction chamber induced by the coil is between 0-200 milliTeslas.

In accordance with the first aspect, the one or more temperature control devices comprise a non-magnetic heating element and/or cooling element configured to maintain a temperature within the reaction chamber between −25° C. to 100° C.

In accordance with an embodiment of the first aspect, the one or more temperature control devices are configured to cycle a temperature within the reaction chamber between at least two different temperatures between −25° C. to 100° C. over a period of time.

In accordance with an embodiment of the first aspect, the one or more reaction chambers are equipped to perform hyperpolarization with a reaction mixture containing the perfluorinated SABRE catalyst, the solvent, and a substrate to be hyperpolarized into the hyperpolarized MRI probe.

In accordance with an embodiment of the first aspect, the solvent is a one phase system or a two phase system comprising water, methanol, ethanol, a fluorous solvent, or a mixture thereof.

In accordance with an embodiment of the first aspect, wherein the one or more MRI probe separators are configured to separate the hyperpolarized MRI probe from the perfluorinated SABRE catalyst by one of filtration; extraction; or column chromatography.

In accordance with an embodiment of the first aspect, the MRI probe infusion device further comprising a gas trap, a gas leak detector, and/or an oxygen level monitor.

In accordance with an embodiment of the first aspect, further comprising a processor configured to execute instructions that cause the processor to: control a flow of gas and/or liquid through the device, monitor a safety metric of the device and/or environment, administer a desired quantity of the hyperpolarized MRI probe to the patient, or calculate a decay rate of the hyperpolarized MRI probe as a function of a rate of flow of the gas and/or the liquid.

In accordance with an embodiment of the first aspect, wherein the one or more MRI probe collectors include one or more dryers.

In accordance with an embodiment of the first aspect, the one or more reaction chambers include at least two reaction chambers configured to be operable in series or in parallel.

In accordance with an embodiment of the first aspect, components of the MRI probe infusion device are made of non-magnetic materials or plastics.

In accordance with a second aspect of the present disclosure, a method is provided of administering a hyperpolarized MRI probe to a patient in need thereof. The method includes:
(i) providing an MRI probe infusion device according to the first aspect,
(ii) supplying to one or more of the reaction chambers a reaction mixture comprising a perfluorinated SABRE catalyst comprising a d-block element and a perfluorinated ligand, a solvent, a co-ligand, and a substrate to be hyperpolarized into an MRI probe,
(iii) agitating the reaction mixture, wherein the agitation is provided via bubbling parahydrogen gas or a mixture of parahydrogen and nitrogen gas through the reaction mixture,
(iv) applying a magnetic field suitable for hyperpolarization of the perfluorinated SABRE catalyst and the substrate to hyperpolarize the substrate into a hyperpolarized MRI probe,
(v) separating the hyperpolarized MRI probe from the reaction mixture by at least one of filtration, extraction, or column chromatography to obtain a solution containing the hyperpolarized MRI probe,
(vi) concentrating the hyperpolarized MRI probe present in the solution obtained in step (v) to obtain a concentrate and reconstituting the concentrate into a solution of desired concentration of the hyperpolarized MRI probe for administering to the patient;
(vii) analyzing at least one of a purity or a concentration of the hyperpolarized MRI probe present in the solution; and
(viii) administering the hyperpolarized MRI probe to the patient.

In accordance with an embodiment of the second aspect, the solvent is selected from a perfluorohexane/diethyl ether mixture, a methoxy nonafluorobutane and ethyl acetate mixture with a non-polar solvent, a perfluorohexane and ether mixture, a perfluorobutyl methyl ether and ethyl acetate mixture, an ether, a fluorocarbon derivative of THF FC 75, a decafluoromethoxy trifluoromethyl pentane, a hexafluoro propanol, a nonafluorobutyl methyl ether, a perfluoromethyl cyclohexane, a perfluoroalkane, a perfluorohexane, and a methoxy nonafluorobutane.

In accordance with an embodiment of the second aspect, the substrate is selected from 1-¹³C-ketoglutarate, 1-¹³C-5-¹²C-ketoglutarate, 1-¹³C-pyruvate, 1-¹³C—N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

In accordance with an embodiment of the second aspect, the perfluorinated ligand is of Formula (I): [Lₘ-(NHC)—(Y—Z)_{q}] or a salt thereof, and wherein:
each L is independently selected from hydrogen, adamantyl, a substituted or unsubstituted aromatic, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4, and
q is an integer from 1 to 3.

In accordance with an embodiment of the second aspect, the perfluorinated tag is one of a perfluorinated C₃₋₆₀ group comprising only carbon and fluorine atoms; a perfluorinated C₃₋₄₀ group comprising only carbon and fluorine atoms; or a perfluorinated C₃₋₂₀ group.

In accordance with an embodiment of the second aspect, the perfluorinated ligand is selected from one of:

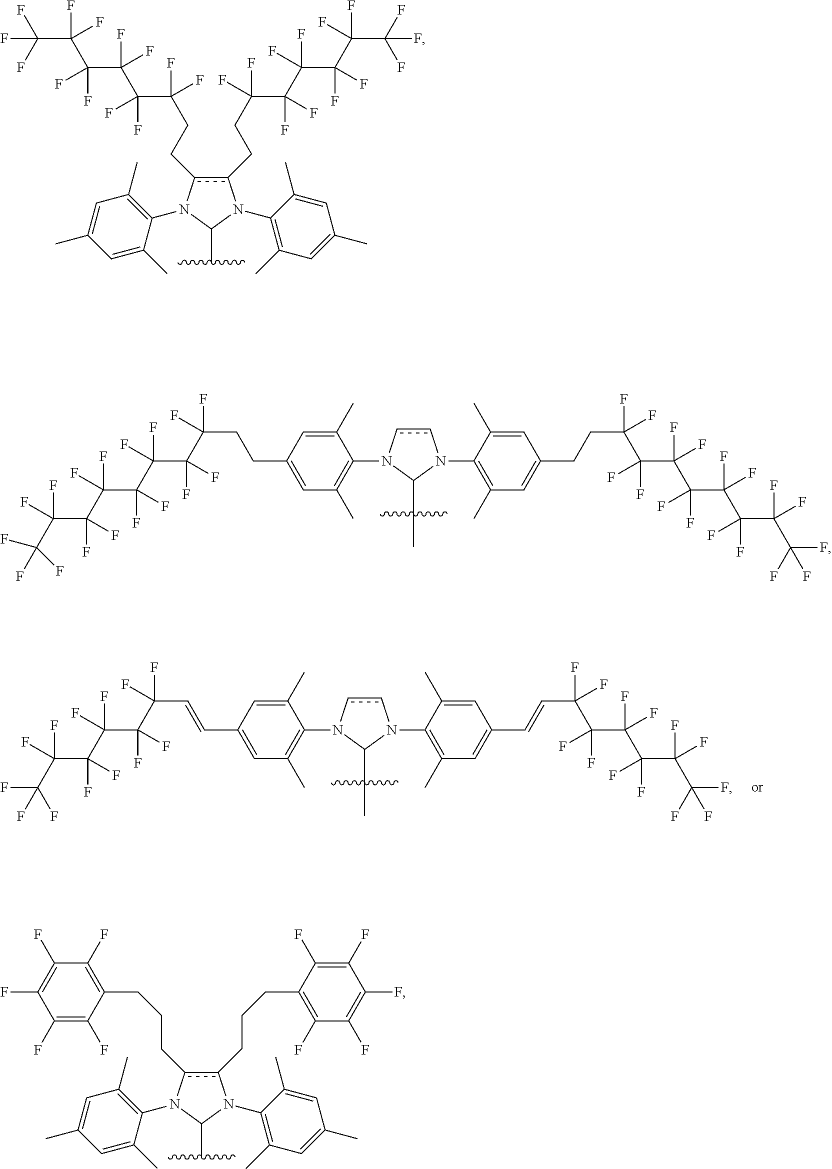

or a salt thereof, and wherein
[symbol] is a single bond or a double bond, and
[symbol] represents the bond to the d-block element via the carbene.

### BRIEF DESCRIPTION OF THE DRAWINGS

FIG. 1 illustrates a reaction chamber and a separator of the hyperpolarized MRI probe, in accordance with an aspect of the disclosure.

FIG. 2 illustrates the internal structure of a reaction chamber, in accordance with an aspect of the disclosure.

FIG. 3 schematically illustrates certain components of the MRI probe infusion device, and their arrangement, in accordance with an aspect of the disclosure.

FIG. 4 illustrates an MRI probe infusion device equipped with multiple reaction chambers and the separation of the hyperpolarization probe from the reaction mixture from each reaction chamber, in accordance with an aspect of the disclosure.

FIG. 5 illustrates some of the steps involved in the method of preparing an MRI probe for administering to a patient, in accordance with an aspect of the disclosure.

FIG. 6 illustrates a fluorous solid phase extraction (F-SPE) process wherein an organic fraction containing a hyperpolarized probe is separated from the fluorous fraction on a fluorous silica gel with a fluorophobic solvent, i.e., by a fluorophilic pass, whereby the hyperpolarized probe is eluted into the fluorous fraction, followed by recovering the catalyst from the fluorous silica gel by a fluorophilic pass, which includes passing aqueous/organic solvent such as methanol, ethanol, ethyl acetate, acetonitrile, or THF, over the silica gel, in accordance with an aspect of the disclosure.

FIG. 7 illustrates the substrate polarization process and the extraction of the probe as illustrated in FIG. 6 by a fluorophilic pass, in accordance with an aspect of the disclosure.

FIG. 8 illustrates a ‘reverse’ F-SPE process wherein the probe is separated from the reaction mixture by a combination of a fluorophilic pass and a fluorophobic process. Thus, the reaction mixture is contacted with a standard silica gel with a fluorophilic solvent, whereby the probe is retained on the silica gel. The probe is then recovered from the silica gel by eluting with a standard organic solvent, or by a fluorophobic pass.

FIG. 9 illustrates the partitioning of the SABRE catalyst and the hyperpolarized substrate between two immiscible phases (fluorous solvent fraction and water or other water immiscible solvent such as chlorinated solvents and water or other hydrophilic solvent) in accordance with an aspect of the disclosure, which allows the principles of phase-transfer catalysis to be employed in conjunction with para hydrogen to produce high levels of hyperpolarization in the aqueous phase without catalyst contamination.

FIG. 10 illustrates the hyperpolarization of a fluorinated SABRE catalyst containing a metal coordinated to a co-ligand and a substrate containing a half spin nucleus to form a hyperpolarized substrate that is free of a metal, in accordance with an aspect of the disclosure.

### DETAILED DESCRIPTION OF THE INVENTION

The present disclosure provides an MRI probe infusion device for producing and administering a hyperpolarized MRI probe (i.e., a hyperpolarized substrate) to a patient. The MRI probe infusion device includes one or more reaction chambers, one or more MRI probe separators, and one or more MRI probe collectors. The MRI probe infusion device may also include a hyperpolarized MRI probe administrator for administering the hyperpolarized MRI probe to a patient. Each reaction chamber may also include:
(a) a structure configured to attenuate a magnetic field within the reaction chamber from an external source, wherein a strength of the magnetic field within the reaction chamber from the external source is less than a threshold value;
(b) one or more inlet ports;
(c) a coil configured to generate an electro-magnetic field within the reaction chamber;
(d) one or more temperature control devices; and
(e) one or more outlet ports.

The present disclosure provides a method for administering the hyperpolarized MRI probe to a subject (e.g., person or animal). The method includes:
(i) providing an MRI probe infusion device according to the first aspect,
(ii) supplying to one or more of the reaction chambers a reaction mixture comprising a perfluorinated SABRE catalyst comprising a d-block element and a perfluorinated ligand, a solvent, a co-ligand, and a substrate to be hyperpolarized into an MRI probe,
(iii) agitating the reaction mixture, wherein the agitation is provided via bubbling parahydrogen gas or a mixture of parahydrogen and nitrogen gas through the reaction mixture,
(iv) applying a magnetic field suitable for hyperpolarization of the perfluorinated SABRE catalyst and the substrate to hyperpolarize the substrate into a hyperpolarized MRI probe,
(v) separating the hyperpolarized MRI probe from the reaction mixture by at least one of filtration, extraction, or column chromatography to obtain a solution containing the hyperpolarized MRI probe,
(vi) concentrating the hyperpolarized MRI probe present in the solution obtained in step (v) to obtain a concentrate and reconstituting the concentrate into a solution of desired concentration of the hyperpolarized MRI probe for administering to the patient;
(vii) analyzing at least one of a purity or a concentration of the hyperpolarized MRI probe present in the solution; and
(viii) administering the hyperpolarized MRI probe to the patient.

The present disclosure also provides a perfluorinated SABRE catalyst introduced to the reaction chamber of at least one MRI probe infusion device, the perfluorinated SABRE catalyst comprising a d-block element and a perfluorinated ligand, wherein the perfluorinated ligand is of Formula (I):

[Lₘ-(NHC)—(Y—Z)_{q}] Formula (I),
or a salt thereof, and wherein
each L is independently selected from hydrogen, adamantyl, a substituted or unsubstituted aromatic group, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4 (e.g., 1, 2, 3, or 4), and
q is an integer from 1 to 3 (e.g., 1, 2, or 3).

The perfluorinated SABRE catalyst comprises a d-block element such as, for example, Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, and/or Hg. In some embodiments, the d-block element is a transition metal such as, for example, Co, Rh, Ir, Ru, Pd, Pt, or Mt. In certain embodiments, the perfluorinated SABRE catalyst comprises an element of group 9 of the periodic table, i.e., Co, Rh, Ir, or Mt. In preferred embodiments, the perfluorinated SABRE catalyst comprises Ir or Co. For example, the perfluorinated SABRE catalyst can be prepared from [Ir(COD)(IMes)(Cl)].

The perfluorinated SABRE catalyst comprises a perfluorinated ligand of Formula (I):

[Lₘ-(NHC)—(Y—Z)_{q}] Formula (I),

or a salt thereof, and wherein
each L is independently selected from hydrogen, adamantyl, a substituted or unsubstituted aromatic group, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4 (e.g., 1, 2, 3, or 4), and
q is an integer from 1 to 3 (e.g., 1, 2, or 3).

In some aspects, NHC comprises an azolyl moiety, i.e., a five membered heterocyclic group having a nitrogen atom and at least one other hetero atom selected from nitrogen, sulfur, and oxygen. Thus, in some embodiments, NHC is a 5-membered N-heterocyclic carbenyl group. For example, the 5-membered N-heterocyclic carbenyl group can be imidazole-based, imidazoline-based, or thiazole-based. In other words, the 5-membered N-heterocyclic carbenyl group can be the resulting carbene formed from treatment of a perfluorinated ligand having an imidazole, an imidazoline, or a thiazole core.

In some embodiments, NHC is a 4,5-disubstituted, a 1,3-disubstituted, or a 1,3,4,5-tetrasubstituted imidazole-based or imidazoline-based 5-membered N-heterocyclic carbenyl group. For example, NHC can be a 4,5-disubstituted imidazolidinyl, a 1,3-disubstituted imidazolidinyl, a 1,3,4,5-tetrasubstituted imidazolidinyl, a 4,5-disubstituted 2,3-dihydro-imidazolyl, a 1,3-disubstituted 2,3-dihydro-imidazolyl, or a 1,3,4,5-tetrasubstituted 2,3-dihydro-imidazolyl. Examples of the imidazolylidinyl moiety include N,N′-di-(2,4,6-trimethylphenyl)-imidazolylidinyl moiety, N,N′-di-(2,6-diisopropylphenyl)-imidazolidinyl moiety, N,N′-di-(2,6-dicyclohexyl)-imidazolidinyl moiety, N,N′-di-(2,6-t-butyl)-imidazolidinyl moiety, and N,N′-di-(1-adamantyl)-imidazolidinyl moiety.

In some embodiments, the perfluorinated ligand is of Formula (Ia) or (Ib):

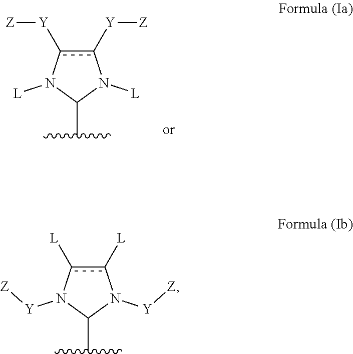

or a salt thereof, and wherein
each L independently is hydrogen, adamantyl, a substituted or unsubstituted aromatic group, or a substituted or unsubstituted heteroaromatic group,
each Y independently is a bond or a spacer group,
each Z independently is a perfluorinated tag,
[symbol] is a single bond or a double bond, and
[symbol] represents the bond to the d-block element via the carbene.

In some embodiments, the perfluorinated ligand is of Formula (Ic) or (Id):

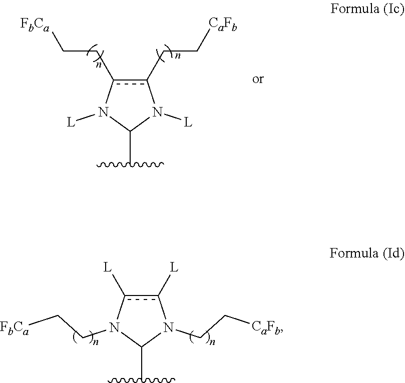

or a salt thereof, and wherein
each L independently is hydrogen, adamantyl, a substituted or unsubstituted aromatic group, or a substituted or unsubstituted heteroaromatic group,
a is 4 to 20 (e.g., 4, 5, 6, 7, 8, 9, 10, 11, 12, 13, 14, 15, 16, 17, 18, 19, or 20),
b=2a+1 or b=a−1,
each n independently is an integer from 0 to 4 (e.g., 0, 1, 2, 3, or 4),
[symbol] is a single bond or a double bond, and
[symbol] represents the bond to the d-block element via the carbene. In some embodiments of Formula (Ic) or (Id) a is 4 to 10 (e.g., 4, 5, 6, 7, 8, 9, or 10).

In some embodiments, the perfluorinated ligand is of Formula (Ie) or (If):

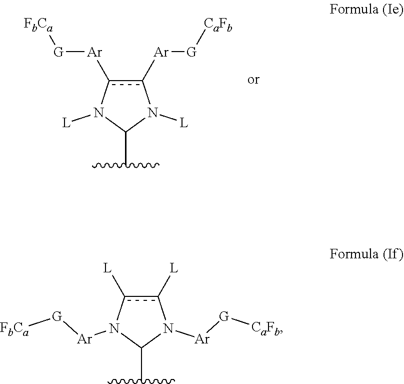

or a salt thereof, and wherein
each L independently is hydrogen, adamantyl, a substituted or unsubstituted aromatic group, or a substituted or unsubstituted heteroaromatic group,
each Ar independently is a substituted or unsubstituted aromatic group, or a substituted or unsubstituted heteroaromatic group,
each G independently is a bond, C₁₋₆ alkyl, C₁₋₆ alkenyl, or C₁₋₆ heteroalkyl,
a is 4 to 20 (e.g., 4, 5, 6, 7, 8, 9, 10, 11, 12, 13, 14, 15, 16, 17, 18, 19, or 20),
b=2a+1 or b=a−1,
[symbol] is a single bond or a double bond, and
[symbol] represents the bond to the d-block element via the carbene. In some embodiments of Formula (Ie) or (If), a is 4 to 10 (e.g., 4, 5, 6, 7, 8, 9, or 10).

In any of Formulae (I) and (Ia)-(If), each L is independently selected from hydrogen, adamantyl, a substituted or unsubstituted aromatic group, or a substituted or unsubstituted heteroaromatic group.

As used herein, “substituted or unsubstituted aromatic” refers to a substituted (e.g., C₁₋₆ alkyl substituted) or unsubstituted aromatic ring having 5 to 60 ring carbon atoms, e.g., phenyl, naphthyl, phenanthryl, and anthracenyl. As used herein, “substituted or unsubstituted heteroaromatic” refers to a substituted (e.g., C₁₋₆ alkyl substituted) or unsubstituted aromatic ring having from 1 to 2 heteroatoms chosen from N, O, and S, with remaining ring atoms being carbon, or a stable bicyclic or tricyclic system containing at least one 5- to 7-membered aromatic ring which contains from 1 to 3, or in some aspects, from 1 to 2, heteroatoms chosen from N, O, and S, with remaining ring atoms being carbon. Monocyclic heteroaryl groups typically have from 5 to 7 ring atoms, in some aspects, bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Heteroaromatic groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, henzofrijoxazolyl, benzofuranyl, benzothiazolyl, benzolhiophenyl, benzoxadiazolyl, dihydrobenzodioxynyl, furanyl, imidazolyl, indolyl, isothiazolyl, and isoxazolyl.

In some embodiments, each L independently is hydrogen, adamantyl, 2-methylphenyl, 3-methylphenyl, 4-methylphenyl, 2,4-dimethylphenyl, 2,5-dimethylphenyl, 2,6-dimethylphenyl, 3,5-dimethylphenyl, 2,4,6-trimethylphenyl, 2-ethylphenyl, 3-ethylphenyl, 4-ethylphenyl, 2,4-diethylphenyl, 2,5-diethylphenyl, 2,6-diethylphenyl, 3,5-diethylphenyl, 2,4,6-triethylphenyl, 2-npropylphenyl, 3-npropylphenyl, 4-npropylphenyl, 2,4-di-npropylphenyl, 2,5-di-npropylphenyl, 2,6-di-npropylphenyl, 3,5-di-npropylphenyl, 2,4,6-tri-npropylphenyl, 2-isopropylphenyl, 3-isopropylphenyl, 4-isopropylphenyl, 2,4-di-isopropylphenyl, 2,5-di-isopropylphenyl, 2,6-di-isopropylphenyl, 3,5-di-isopropylphenyl, 2,4,6-tri-isopropylphenyl, 2-isobutylphenyl, 3-isobutylphenyl, 4-isobutylphenyl, 2,4-di-isobutylphenyl, 2,5-di-isobutylphenyl, 2,6-di-isobutylphenyl, 3,5-di-isobutylphenyl, 2,4,6-tri-isobutylphenyl, 2-secbutylphenyl, 3-secbutylphenyl, 4-secbutylphenyl, 2,4-di-secbutylphenyl, 2,5-di-secbutylphenyl, 2,6-di-secbutylphenyl, 3,5-di-secbutylphenyl, 2,4,6-tri-secbutylphenyl, 2-tbutylphenyl, 3-tbutylphenyl, 4-tbutylphenyl, 2,4-di-tbutylphenyl, 2,5-di-tbutylphenyl, 2,6-di-tbutylphenyl, 3,5-di-tbutylphenyl, 2,4,6-tri-tbutylphenyl, 2-cyclohexylphenyl, 3-cyclohexylphenyl, 4-cyclohexylphenyl, 2,4-di-cyclohexylphenyl, 2,5-di-cyclohexylphenyl, 2,6-di-cyclohexylphenyl, 3,5-di-cyclohexylphenyl, or 2,4,6-tri-cyclohexylphenyl. In certain embodiments of Formulae (I) and (Ia)-(If), each L independently is hydrogen or 2,4,6-trimethylphenyl.

In any of Formulae (I) and (Ia)-(If), each Y independently is a bond or a spacer group. For example, Y can be a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group (e.g., polyethylene oxide, polypropylene oxide, or a combination thereof).

In some embodiments, each Y independently is a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group. In certain embodiments, each Y independently is a bond, a substituted or unsubstituted C₁₋₁₀ alkyl group, a substituted or unsubstituted C₂₋₁₀ alkenyl group, a substituted or unsubstituted aryl group, a substituted or unsubstituted heteroaryl group, a substituted or unsubstituted alkaryl group, or a substituted or unsubstituted arylalkyl group. In preferred embodiments, each Y independently is a bond, a substituted or unsubstituted C₁₋₁₀ alkyl group, a substituted or unsubstituted C₂₋₁₀ alkenyl group, a substituted or unsubstituted aryl group, a substituted or unsubstituted heteroaryl group, a substituted or unsubstituted alkaryl group, or a substituted or unsubstituted arylalkyl group.

The perfluorinated ligand comprises a perfluorinated tag. For example, in any of Formulae (I) and (Ia)-(If), each Z is a perfluorinated tag. The perfluorinated tag can be any perfluorinated group such as, for example, a perfluorinated alkyl (e.g., linear or branched), aryl, alkyarl, or arylalkyl group containing up to 60 carbon atoms. In some embodiments, the perfluorinated tag is a perfluorinated C₃₋₆₀ group comprising only carbon and fluorine atoms. In certain embodiments, the perfluorinated tag is a perfluorinated C₃₋₄₀ group comprising only carbon and fluorine atoms. In other embodiments, the perfluorinated tag is a perfluorinated C₃₋₂₀ group. For example, the perfluorinated tag can be selected from a C₄F₉ group, a C₅F₁₁ group, a C₆F₁₃ group, a C₇F₁₅ group, a C₈F₁₇ group, a C₉F₁₉ group, a C₁₀F₂₁ group, a C₆F₅ group, C₄F₇ group, a C₅F₉ group, a C₆F₁₁ group, a C₇F₁₃ group, a C₈F₁₅ group, a C₉F₁₇ group, and a C₁₀F₁₉ group, each of which can be a linear or branch alkyl, aryl, alkyarl, or arylalkyl group.

In an aspect, Z is a perfluoroalkyl chain, linear or branched, having a chain length of up to 60 or more carbon atoms, for example, the perfluoroalkyl chain has a chain length of 3-60, particularly, 3 to 40, more particularly 3 to 20, and even more particularly 3 to 10 or more, carbon atoms. For example, the perfluoroalkyl chain is selected from the group consisting of C₄F₉, C₆F₁₃, C₇F₁₅, C₈F₁₇, C₉F₁₉, and C₁₀F₂₁, preferably selected from the group consisting of C₆F₁₃, C₈F₁₇, and C₁₀F₂₁, each of which can be linear or branched and combinations thereof, wherein each of which can be linear or branched.

In an aspect, the perfluorinated ligand is of formula (Ig):

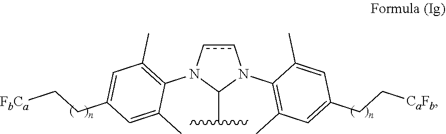

or a salt thereof, and wherein
a is 4 to 20 (e.g., 4, 5, 6, 7, 8, 9, 10, 11, 12, 13, 14, 15, 16, 17, 18, 19, or 20),
b=2a+1 or b=a−1,
[symbol] is a single bond or a double bond, and
[symbol] represents the bond to the d-block element via the carbene. In some embodiments of Formula (Ig), a is 4 to 10 (e.g., 4, 5, 6, 7, 8, 9, or 10).

In an aspect, the perfluorinated ligand is of formula (Ih):

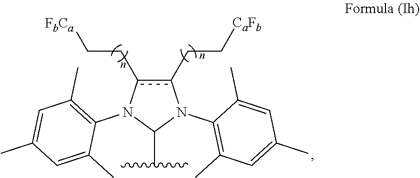

or a salt thereof, and wherein
a is 4 to 20 (e.g., 4, 5, 6, 7, 8, 9, 10, 11, 12, 13, 14, 15, 16, 17, 18, 19, or 20),
b=2a+1 or b=a−1,
[symbol] is a single bond or a double bond, and
[symbol] represents the bond to the d-block element via the carbene. In some embodiments of Formula (Ig), a is 4 to 10 (e.g., 4, 5, 6, 7, 8, 9, or 10).

Exemplary perfluorinated ligands include:

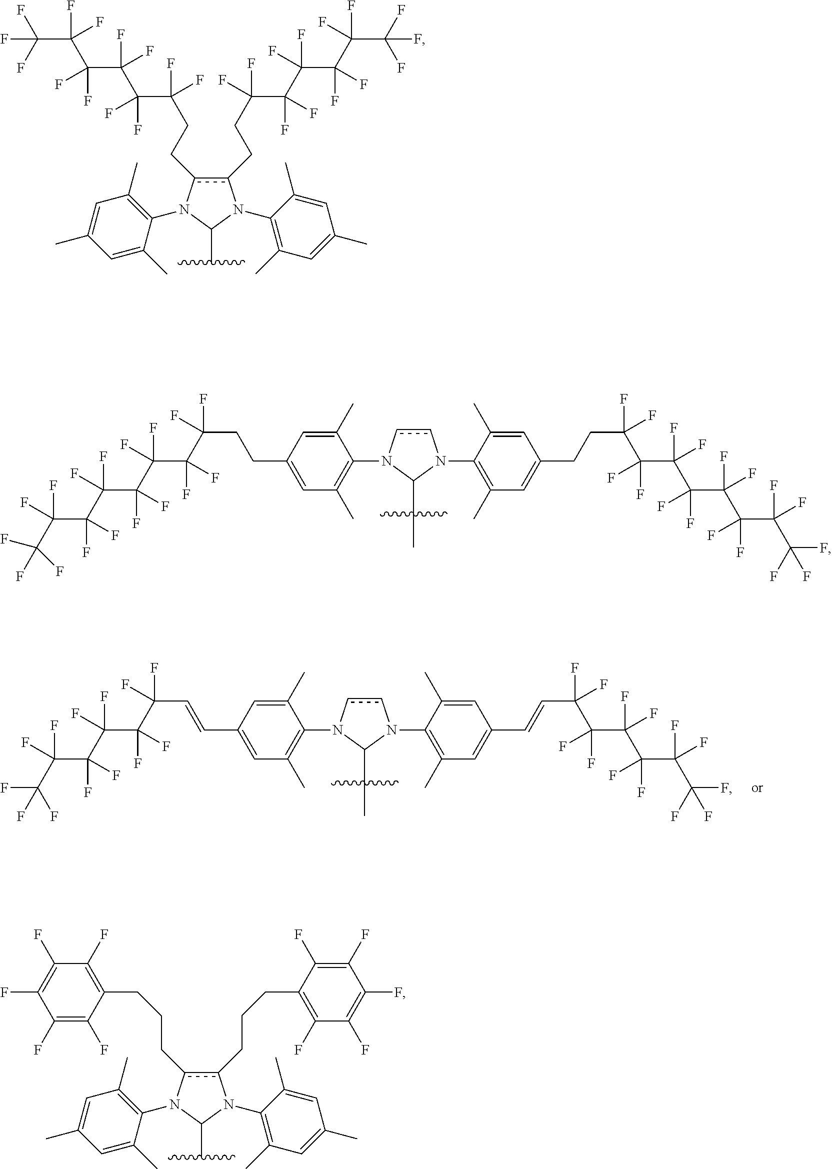

or a salt thereof, and wherein [symbol] is a single bond or a double bond, and [symbol] represents the bond to the d-block element via the carbene.

As used herein, the symbol “ [symbol] ” represents a single bond or a double bond. In some embodiments, [symbol] is a single bond. In other embodiments, [symbol] is a double bond. In embodiments where [symbol] is a single bond, the orientation of the two substituents stemming from [symbol] can have any suitable stereochemistry, i.e., can be cis or trans. In preferred embodiments, when [symbol] is a single bond, the stereochemistry of the substituents stemming from [symbol] is trans.

As used herein, the symbol “ [symbol] ” represents the bond to the d-block element via the carbene. In other words, [symbol] represents the bond to the metal of the catalyst.

In some embodiments, the perfluorinated SABRE catalyst further comprises an additional ligand. For example, the perfluorinated SABRE catalyst may further comprise an additional ligand selected from phosphine ligands, carbene ligands, imidazole ligands, pincer chelating ligands, and compounds comprising a sulfoxide group. In certain embodiments, the perfluorinated SABRE catalyst comprises one or more phosphine ligands. Examples of phosphine ligands include, but are not limited to the following:

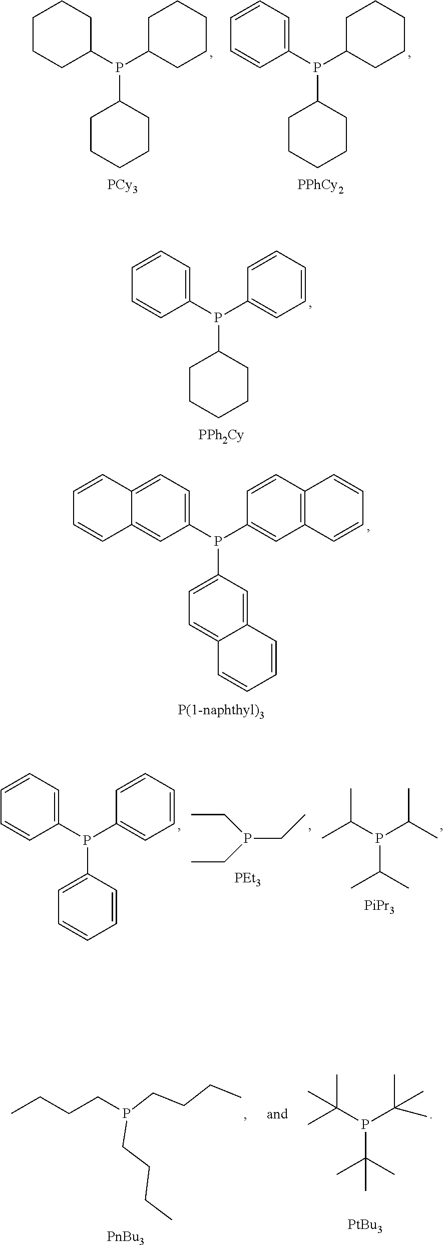

In some embodiments, the perfluorinated SABRE catalyst comprises a pincer chelating ligand. Generally, when the perfluorinated SABRE catalyst comprises a phosphine ligand or a pincer chelating ligand, the perfluorinated SABRE catalyst is in pre-catalyst form. In some embodiments, the perfluorinated SABRE catalyst comprises a ligand that is a compound comprising a sulfoxide group. Examples of compounds comprising a sulfoxide group can be selected from the group consisting of dimethylsulfoxide (DMSO), phenyl trifluoromethyl sulfoxide, phenyl methyl sulfoxide, phenyl chloromethyl sulfoxide, diphenyl sulfoxide, dibenzoyl sulfoxide, and dibutyl sulfoxide. Generally, when the perfluorinated SABRE catalyst comprises a compound comprising a sulfoxide group, the perfluorinated SABRE catalyst is in active form. As used herein, “the perfluorinated SABRE catalyst” can refer to the active perfluorinated SABRE catalyst or the perfluorinated SABRE precatalyst.

The active perfluorinated SABRE catalyst can be prepared by any suitable method. Generally, the active perfluorinated SABRE catalyst is prepared by combining the perfluorinated SABRE precatalyst with a substrate, parahydrogen, and optionally a co-ligand in a solvent to form a mixture comprising an active perfluorinated SABRE catalyst. In some embodiments, the active perfluorinated SABRE catalyst is prepared by combining the perfluorinated SABRE precatalyst with a substrate, parahydrogen, and a co-ligand in a solvent to form a mixture comprising an active perfluorinated SABRE catalyst. The co-ligand, when included in the preparation of the active perfluorinated SABRE catalyst, can be combined with the perfluorinated SABRE precatalyst in any order and by any suitable means. For example, the co-ligand, when included in the preparation of the active SABRE catalyst, can be provided first to interact with the transfer precatalyst to facilitate formation of the active perfluorinated SABRE catalyst. Alternatively, the co-ligand, when included in the preparation of the active perfluorinated SABRE catalyst, can be added together with the substrate to facilitate formation of the active perfluorinated SABRE catalyst. In some embodiments, the co-ligand, the substrate, and parahydrogen are essentially combined with the perfluorinated SABRE precatalyst in the solvent at the same time to facilitate formation of the active perfluorinated SABRE catalyst. In other embodiments, the substrate is provided first to interact with the perfluorinated SABRE precatalyst to facilitate formation of the active SABRE catalyst. In some embodiments, the co-ligand and the substrate are combined with the perfluorinated SABRE precatalyst in the solvent, and the parahydrogen is added to (e.g., bubbled through) the resulting mixture. In other embodiments, the substrate is combined with the perfluorinated SABRE precatalyst in the solvent, and the parahydrogen is added to (e.g., bubbled through) the resulting mixture. In some embodiments, the active perfluorinated SABRE catalyst is prepared by combining the perfluorinated SABRE precatalyst with a co-ligand in addition to the substrate and parahydrogen.

In some embodiments, the active perfluorinated SABRE catalyst is of formula

[Ir(H)₂(F-IMes)(η²-SUBSTRATE)(Co-ligand)] or

[Ir(H)₂(F-IMes)(η¹-SUBSTRATE)(Co-ligand)₂],

wherein SUBSTRATE is a target substrate to be hyperpolarized by the transfer of the pure singlet spin order of parahydrogen by the spin transfer catalyst, preferably a target substrate having enriched with an atom having ½ spin nuclei, for example, ¹H, ¹³C, ¹⁵N, ¹⁹F, ³¹P and/or ²⁹Si. Without wishing to be bound by any particular theory, the co-ligand interacts with the spin transfer pre-catalyst to form the activated polarization transfer catalyst and enhances the polarization transfer to the target substrate. F-IMes refers to a perfluorinated form of N-heterocyclic carbenyl (NHC) ligand such as 1,3-Bis(2,4,6-trimethylphenyl)-1,3-dihydro-2H-imidazol-2-ylidene group.

In some embodiments, the perfluorinated SABRE catalyst (e.g., the perfluorinated SABRE precatalyst) can be prepared by a method comprising reacting a perfluorinated compound with a base to form a carbene, and reacting the carbene with [(d-block element)(COD)Cl]₂, wherein COD stands for cyclooctadienyl. In certain embodiments, the method comprises reacting a perfluorinated compound with a base to form a carbene, and reacting the carbene with [Ir(COD)Cl]₂. For example, a perfluorinated SABRE catalyst of formula:

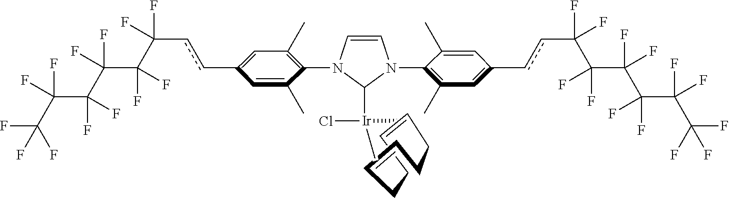

can be prepared by reacting a carbene of formula:

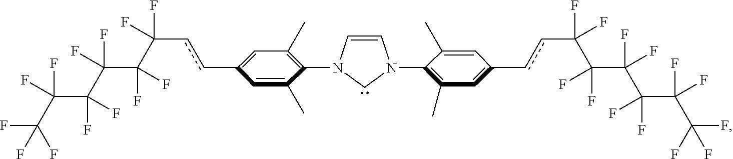

with [Ir(COD)Cl]₂.

Exemplary perfluorinated SABRE catalysts (e.g., the perfluorinated SABRE precatalyst) include:

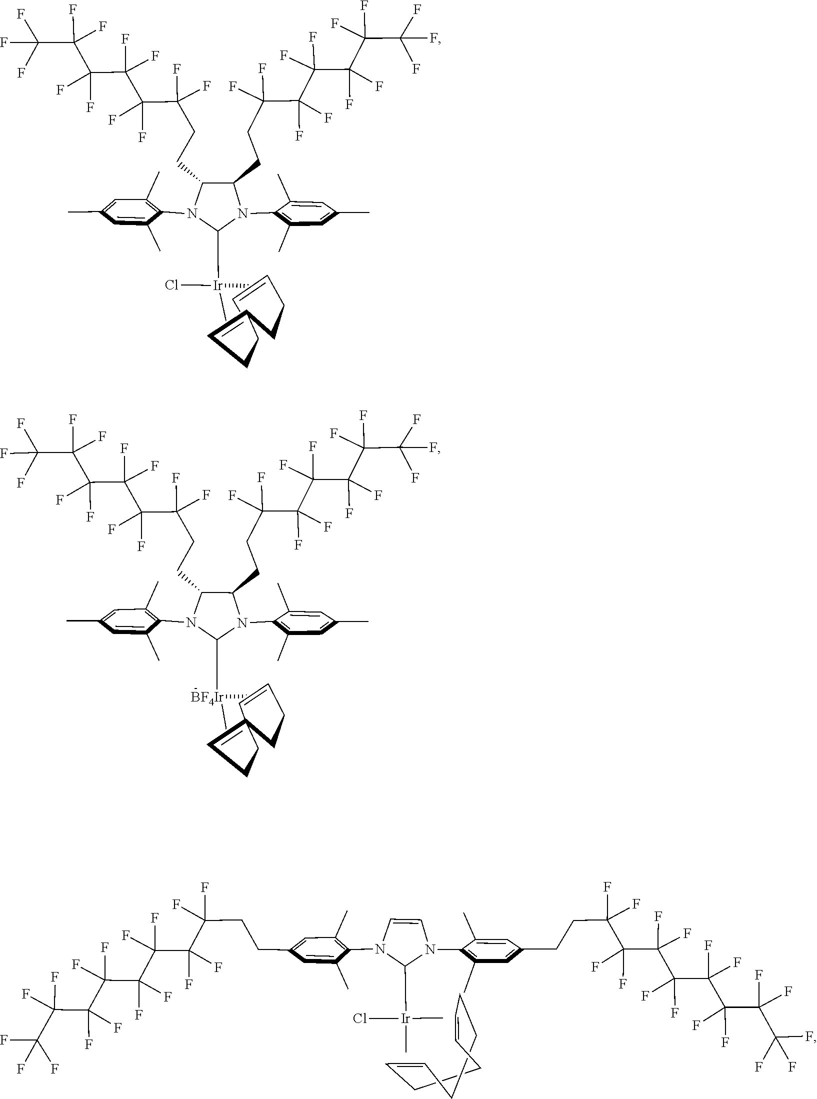

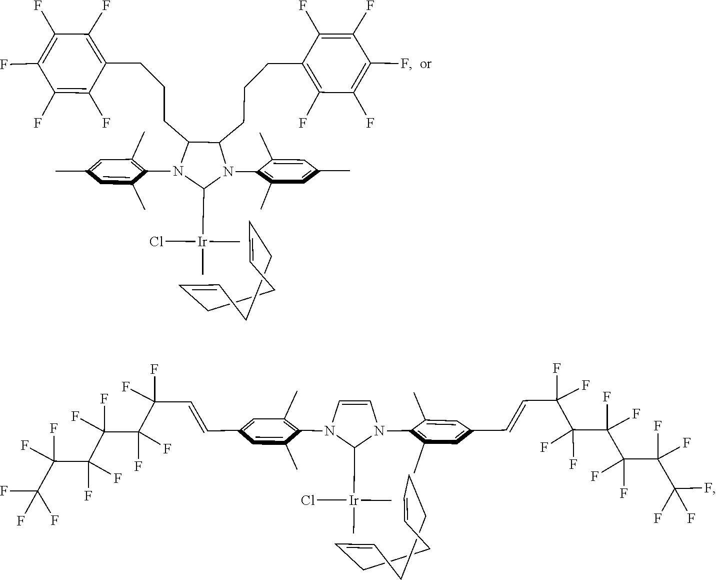

or salts thereof.

As used herein, the terms “hyperpolarized substrate” and “hyperpolarized MRI probe” are used interchangeably to refer to the desired hyperpolarized compound.

The methods of preparing a hyperpolarized substrate, described herein, may comprise hyperpolarizing the mixture comprising the perfluorinated SABRE catalyst (e.g., the active perfluorinated SABRE catalyst) by exposing the mixture to a magnetic field or radiofrequency excitation to transfer the polarization from parahydrogen to the substrate to form the hyperpolarized substrate. Initially, the hyperpolarized substrate is complexed with the hyperpolarized perfluorinated SABRE catalyst; however, it will be understood by a person of ordinary skill in the art that the hyperpolarized substrate can be replaced by another substrate molecule such that the process can be repeated and the free hyperpolarized substrate bolus is produced.

The transfer of polarization from parahydrogen to the substrate to form the hyperpolarized substrate can occur under any suitable magnetic field or radiofrequency excitation. For example, the transfer of polarization from parahydrogen to the substrate can occur at a magnetic field below the magnetic field of earth. The suitable level of magnetic field or radiofrequency excitation necessary to transfer the polarization from parahydrogen to the substrate to form the hyperpolarized substrate will be readily apparent to a person of ordinary skill in the art.

In some embodiments, the method comprises replenishing the parahydrogen in the mixture during the step of hyperpolarizing the mixture comprising the perfluorinated SABRE catalyst (e.g., the active perfluorinated SABRE catalyst) by exposing the mixture to a magnetic field or radiofrequency excitation to transfer the polarization from parahydrogen to the substrate to form the hyperpolarized substrate. In other words, in some embodiments, the method comprises bubbling parahydrogen through the mixture comprising the perfluorinated SABRE catalyst (e.g., the active perfluorinated SABRE catalyst) during the step of hyperpolarizing the mixture comprising the perfluorinated SABRE catalyst (e.g., the active perfluorinated SABRE catalyst) by exposing the mixture to a magnetic field or radiofrequency excitation to transfer the polarization from parahydrogen to the substrate to form the hyperpolarized substrate.

The method of preparing a hyperpolarized substrate may comprise providing a co-ligand to interact with the perfluorinated SABRE catalyst to facilitate formation of an active perfluorinated SABRE catalyst. The co-ligand can be any suitable compound containing one or more sulfoxide groups, thioester groups, phosphine groups, amine groups, CO groups, isonitrile groups, nitrogen-containing heterocyclic groups, or a combination thereof. In some embodiments, the co-ligand is a compound comprising a sulfoxide group. Examples of compounds comprising a sulfoxide group can be selected from the group consisting of DMSO, phenyl methyl sulfoxide, phenyl chloromethyl sulfoxide, diphenyl sulfoxide, dibenzoyl sulfoxide, phenyl trifluoromethyl sulfoxide, and dibutyl sulfoxide. In certain embodiments, the co-ligand is dimethyl sulfoxide or phenyl trifluoromethyl sulfoxide.

In some embodiments of the method of preparing a hyperpolarized substrate, the magnetic field is an electro-magnetic field. For example, the strength of the electro-magnetic field can be in the range of 0-200 milliTeslas (mT). In some embodiments, the electro-magnetic field may be at least partially supplied by one or more permanent magnets in addition to or in lieu of the coil. In some embodiments, the electro-magnetic field may be an alternating magnetic field supplied at a frequency adapted to a particular nuclei. The alternating magnetic field can change directions (i.e., alternate between positive and negative relative to a positive direction). In some embodiments, the frequency can be a radio frequency, and preferably between 50 to 500 MHz, although other frequencies outside this range are contemplated as within the scope of the present disclosure.

The perfluorinated SABRE catalyst (e.g., active perfluorinated SABRE catalyst) is combined with parahydrogen and a substrate comprising a ½ spin nucleus or nuclei in a solvent to obtain a reaction mixture. The solvent can be any suitable solvent capable of forming a heterogeneous or homogeneous mixture. In some embodiments, the solvent comprises water, methanol, ethanol, a fluorous solvent, or a mixture thereof. For example, the solvent can be ethanolic or methanolic, i.e., comprising at least ethanol or methanol in combination with water. In certain embodiments, the solvent comprises a fluorous solvent. In other embodiments, the solvent is deuterated such that a deuterated solvent can be prepared without (i.e., with limited) deuterium-hydrogen exchange.

The fluorous solvent can be any organic solvent comprising at least one compound having a fluorine atom. Without wishing to be bound by any particular theory, it is believed that the fluorous solvent increases the solubility of the perfluorinated SABRE catalyst. In some embodiments, the solvent (e.g., the fluorous solvent) is selected from a perfluorohexane/diethyl ether mixture, a methoxy nonafluorobutane and ethyl acetate mixture with a non-polar solvent, a perfluorohexane and ether mixture, a perfluorobutyl methyl ether and ethyl acetate mixture, an ether, a fluorocarbon derivative of THF FC 75, a decafluoromethoxy trifluoromethyl pentane, a hexafluoro propanol, a nonafluorobutyl methyl ether, a perfluoromethyl cyclohexane, a perfluoroalkane, a perfluorohexane, and a methoxy nonafluorobutane.

In some embodiments, the method of preparing a hyperpolarized substrate further comprises isolating the hyperpolarized substrate. The hyperpolarized substrate can be isolated by any suitable method. For example, the hyperpolarized substrate can be isolated by extraction, filtration, column chromatography, distillation, crystallization, or a combination thereof.

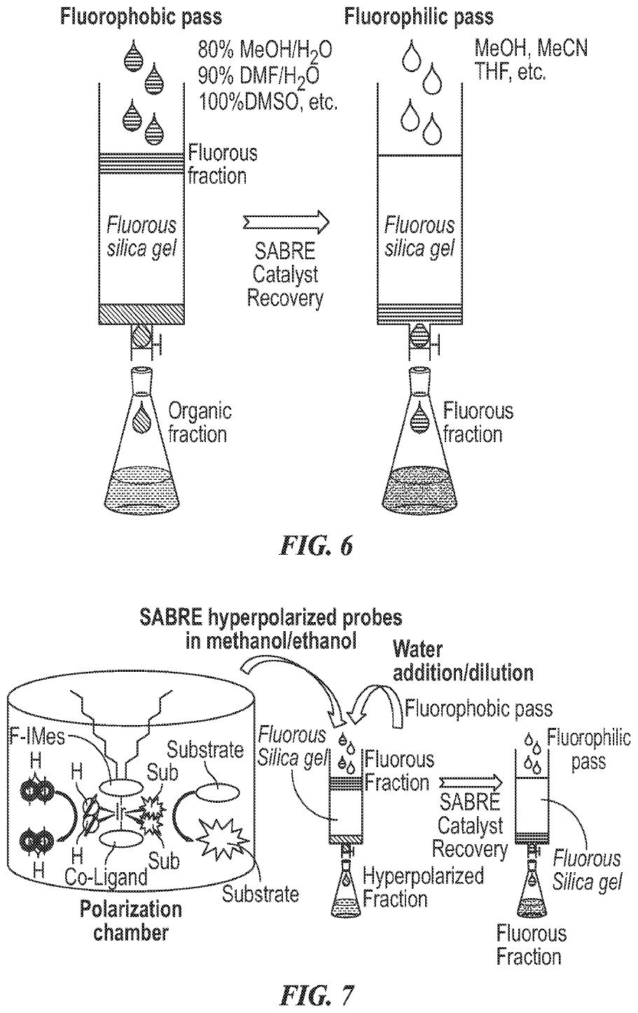

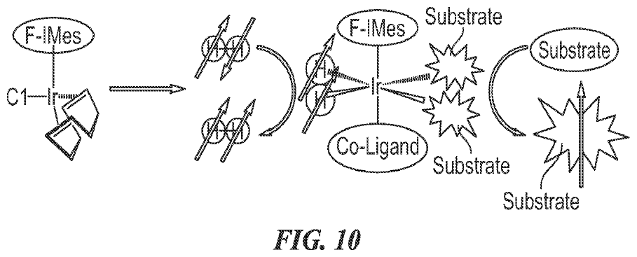

In some embodiments, the hyperpolarized substrate is isolated by treating the reaction mixture with a solid phase adsorbent to adsorb the perfluorinated SABRE catalyst, and recovering a liquid containing the hyperpolarized substrate, wherein the liquid is free (i.e., undetectable) or substantially free (e.g., less than 100 ppm, less than 50 ppm, less than 10 ppm, less than 5 ppm, or less than 1 ppm) of the perfluorinated SABRE catalyst. See, for example, FIG. 10. The solid phase adsorbent can be any suitable adsorbent capable of preferentially adsorbing the perfluorinated SABRE catalyst over the hyperpolarized substrate. For example the solid phase adsorbent can be a fluorous solid phase adsorbent, a reverse phase adsorbent (e.g., C18 adsorbents or the like), and polyethylene-based filters (e.g., ultrahigh molecular weight polyethylene). See, for example, FIGS. 6 and 7. In certain embodiments, the method of preparing a hyperpolarized substrate further comprises passing a fluorophobic solvent over the adsorbent and recovering an eluate containing the hyperpolarized substrate, wherein the eluate is free (i.e., undetectable) or substantially free (e.g., less than 100 ppm, less than 50 ppm, less than 10 ppm, less than 5 ppm, or less than 1 ppm) of the perfluorinated SABRE catalyst. The fluorophobic solvent can be any suitable solvent capable of preferentially washing the hyperpolarized substrate off of the solid phase adsorbent relative to the perfluorinated SABRE catalyst. For example, the fluorophobic solvent can comprise water and one or more of methanol, ethanol, acetonitrile, and dimethylformamide. Alternatively, or additionally, the method of preparing a hyperpolarized substrate can further comprise passing a fluorophilic solvent (e.g., a solvent comprising an organic solvent selected from methanol, ethanol, acetonitrile, THF, ethyl acetate, a chlorinated solvent (e.g., chlorinated alkanes such as methylene chloride, chloroform, and ethylene dichloride), and a combination thereof) over the adsorbent, for example, to recover the perfluorinated SABRE catalyst. Exemplary fluorophilic solvent systems include a perfluorohexane/diethyl ether mixture, a methoxy nonafluorobutane and ethyl acetate mixture, a perfluorohexane and diethyl ether mixture, a perfluorobutyl methyl ether and ethyl acetate mixture, or a diethyl ether.

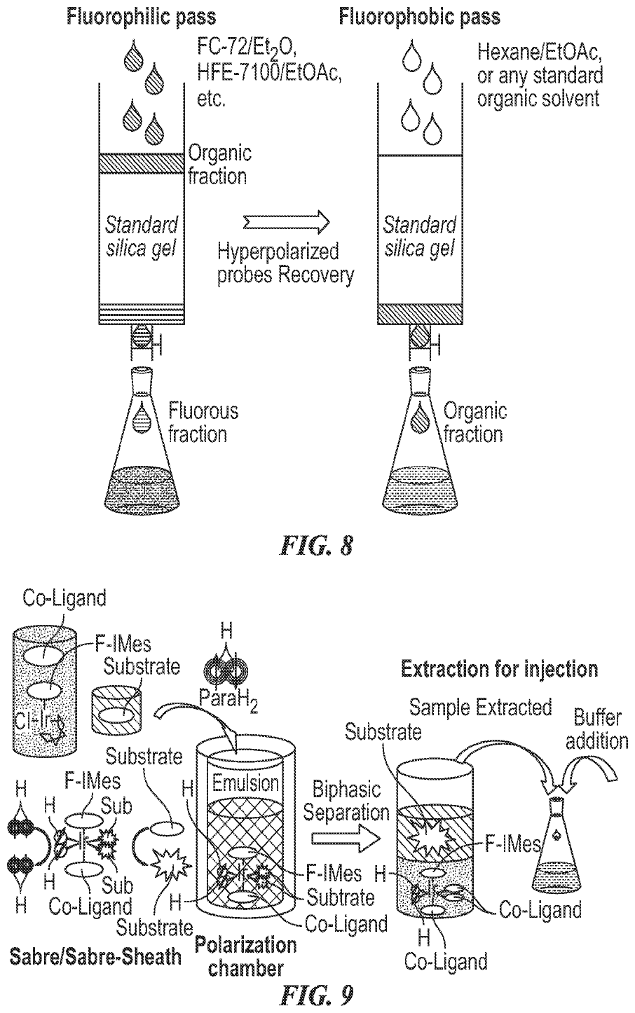

In some embodiments, the hyperpolarized substrate is isolated by treating the reaction mixture with a solid phase adsorbent to adsorb the hyperpolarized substrate, and recovering a liquid containing the perfluorinated SABRE catalyst, wherein the liquid is free (i.e., undetectable) or substantially free (e.g., less than 100 ppm, less than 50 ppm, less than 10 ppm, less than 5 ppm, or less than 1 ppm) of the hyperpolarized substrate. The solid phase adsorbent can be any suitable adsorbent capable of preferentially adsorbing the hyperpolarized substrate over the perfluorinated SABRE catalyst. For example the solid phase adsorbent can be a normal phase adsorbent such as, for example silica, alumina, or the like. See, for example, FIG. 8. In certain embodiments, the method of preparing a hyperpolarized substrate further comprises passing a fluorophilic solvent over the adsorbent and recovering an eluate containing the perfluorinated SABRE catalyst, wherein the eluate is free (i.e., undetectable) or substantially free (e.g., less than 100 ppm, less than 50 ppm, less than 10 ppm, less than 5 ppm, or less than 1 ppm) of the hyperpolarized substrate. The fluorophilic solvent can be any suitable solvent capable of preferentially washing the perfluorinated SABRE catalyst off of the solid phase adsorbent relative to the hyperpolarized substrate. For example, the fluorophobic solvent can comprise a perfluorohexane/diethyl ether mixture, a methoxy nonafluorobutane and ethyl acetate mixture, a perfluorohexane and diethyl ether mixture, a perfluorobutyl methyl ether and ethyl acetate mixture, or a diethyl ether. Alternatively, or additionally, the method of preparing a hyperpolarized substrate can further comprise passing a fluorophobic solvent (e.g., a solvent comprising water, methanol, ethanol, acetonitrile, dimethylformamide, or a combination thereof) over the adsorbent, for example, to recover the hyperpolarized substrate.

In any of the embodiments disclosed herein, the perfluorinated SABRE catalyst and/or the hyperpolarized substrate can exist in a monophasic or a biphasic mixture. The monophasic or biphasic mixture can comprise any combination of solvents described herein. For example, the biphasic mixture can comprise a polar solvent (e.g., water, methanol, and ethanol) in combination with a non-polar solvent (e.g., an organic solvent or a fluorous solvent). In embodiments, where the perfluorinated SABRE catalyst and/or the hyperpolarized substrate exists in a biphasic solvent, the perfluorinated SABRE catalyst and/or the hyperpolarized substrate can be isolated by a liquid/liquid extraction. See, for example, FIG. 9. Thus, in some embodiments, the hyperpolarized substrate is isolated by a liquid/liquid extraction, for example, by partitioning the perfluorinated SABRE catalyst and the hyperpolarized substrate between a methanolic mixture and a fluorous solvent or partitioning the perfluorinated SABRE catalyst and the hyperpolarized substrate between a methanolic mixture and an organic solvent.

In some embodiments, the hyperpolarized substrate is isolated by precipitating the perfluorinated SABRE catalyst and filtering and removing the precipitated perfluorinated SABRE catalyst from the hyperpolarized substrate. Typically, the perfluorinated SABRE catalyst is precipitated by addition of solvents in which the perfluorinated SABRE catalyst is not soluble (e.g., hexane, pentane, water, ethanol, or the like). In certain embodiments, the perfluorinated SABRE catalyst is precipitated by the addition of water.

The perfluorinated SABRE catalyst and/or the hyperpolarized substrate can be dried or concentrated (e.g., under reduced pressure, using a desiccant, heating, or a combination thereof). Alternatively, or additionally, the perfluorinated SABRE catalyst and/or the hyperpolarized substrate can be diluted or reconstituted with a solvent (e.g., water) to provide a desired concentration. For example, the perfluorinated SABRE catalyst can be isolated and re-used as a hyperpolarization catalyst. Similarly, the hyperpolarized substrates can be dried or concentrated to remove organic solvents and reconstituted in water for administration to a subject.

The substrate can be any compound comprising a 12 spin nucleus or nuclei. For example, the substrate can comprise ¹H, ¹³C, ¹⁵N, ¹⁹F, ³¹P, ²⁹Si, or a combination thereof. In some embodiments, the substrate further comprises ²D. Thus, the methods described herein can be used to enhance the signal of ¹H, ¹³C, ¹⁵N, ¹⁹F, ³¹P and/or ²⁹Si response of a target substrate. Generally, the spin polarization transfer described herein is based on the SABRE effect; however the methods can be extended to parahydrogen-induced polarization (PHIP).

In some embodiments, the substrate is selected from ketoglutarate, pyruvate, N-acetyl cysteine, and salts or esters thereof. In certain embodiments, the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

In some embodiments, the substrate is of Formula (II):

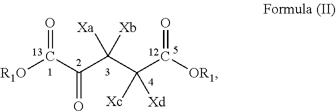

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

Each R₁ may be independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl. In some embodiments, each R₁ is independently selected from a C₁-C₆ alkyl, for example, each R₁ can be methyl, ethyl, propyl (e.g., isopropyl or n-propyl), butyl (e.g., isobutyl, n-butyl, tert-butyl, or sec-butyl), pentyl, or hexyl. In some embodiments, each R₁ is independently selected from hydrogen, deuterium, and a cation. In embodiments where R₁ is a cation, it will be readily understood by a person of ordinary skill in the art that the compound of Formula (II) is a salt (e.g., a pharmaceutically acceptable salt) where the negative charge on oxygen is balanced by the cation. In certain embodiments, each R₁ independently is a cation or C₁-C₆ alkyl.

The present disclosure further provides a hyperpolarized substrate, or a pharmaceutically acceptable salt, obtained from any of the methods described herein, or a pharmaceutical composition comprising a hyperpolarized substrate, or a pharmaceutically acceptable salt, and a pharmaceutically acceptable carrier. In other words, the present invention provides imaging medium (e.g., an aqueous imaging composition) with enhanced sensitivity on a water-soluble compound comprising a hyperpolarizable nucleus or hyperpolarizable nuclei, which imaging medium is particularly well suited for nuclear magnetic resonance (NMR) spectroscopy and/or magnetic resonance imaging (MRI).

The present disclosure further provides a method of obtaining a magnetic resonance image of a tissue in a subject having or suspected to have a cancer or an adverse vascular condition comprising administering to the subject a hyperpolarized substrate described herein, or a pharmaceutical composition thereof, and imaging the subject by magnetic resonance imaging. In an aspect, the subject has a cancer such as, for example, a cancer is selected from breast cancer, colon cancer, rectal cancer, bladder cancer, endometrial cancer, kidney cancer, lung cancer, melanoma, non-Hodgkin lymphoma, pancreatic cancer, prostate cancer, and thyroid cancer. In another aspect, the subject has an adverse vascular condition such as, for example, a vascular condition selected from myocardial infarction, stroke, and pulmonary disease (e.g., COPD, lung fibrosis, long-term COVID-19 symptom, and combinations thereof).

In some embodiments, the disclosure provides a method of diagnosing or monitoring a patient having or suspected to have a cancer, the method comprising administering a hyperpolarized substrate or a pharmaceutical composition as described above and diagnosing or monitoring the patient by hyperpolarized ¹³C-MRI. For example, a hyperpolarized substrate can be used in the method of diagnosing or monitoring a patient having or suspected to have a cancer. In certain embodiments, the method or use comprises identifying a mutation or mutations responsible for the cancer. In certain embodiments, the method or use identifies an IDH1 mutation as being responsible for the cancer. In other words, the method or use can be used to identify whether the patient has a tumor, for example, an IDH1 mutation.

The figures will now be described to illustrate various aspects of the disclosed MRI probe infusion device. It will be appreciated that the features of any one aspect of the present disclosure are not intended to limit the scope of the other aspects of the present disclosure, and that features from multiple aspects disclosed herein can be combined unless contradicted by the context.

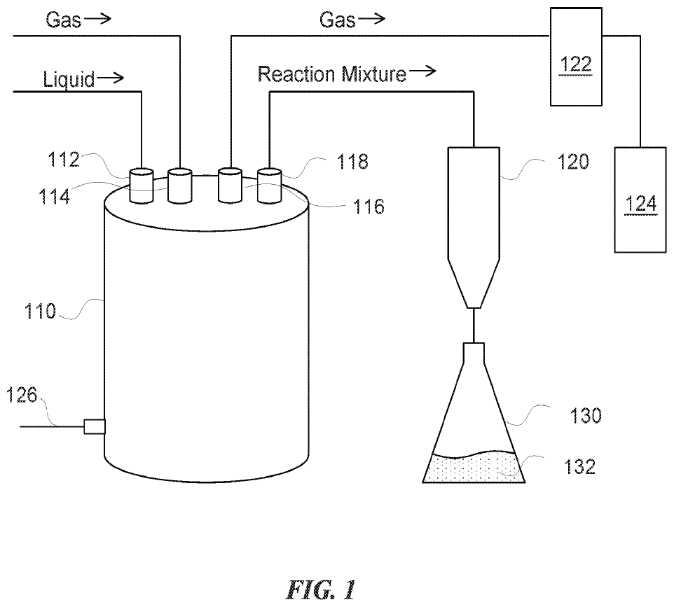

FIG. 1 illustrates the reaction chamber 110 and separator 120 of the hyperpolarized MRI probe infusion device, in accordance with an aspect of the disclosure. As shown in FIG. 1, the reaction chamber 110 includes a number of inlet ports 112, 114, and a number of outlet ports 116, 118. In accordance with an aspect, the reaction chamber 110 includes a first inlet port 112 for receiving a liquid input (e.g., solvent, catalyst, substrate, etc.) and a second inlet port 114 for receiving a gas input (e.g., para-Hydrogen gas). The reaction chamber 110 also includes a first outlet port 116 for venting gas from the reaction chamber 110 and a second outlet port 118 for drawing a reaction mixture from the reaction chamber 110 into the separator 120.

In accordance with an aspect, the first inlet port 112 may introduce a solution including one or more solvents, a catalyst, and a substrate into the reaction chamber. The solvents can include ethanol, a mixture of ethanol and a fluorous solvent, methanol, or a mixture of methanol and a fluorous solvent, for example, the one or more solvents comprise ethanol and water. The substrate can include an element having 12 spin nucleus selected from the group consisting of ¹³C, ¹⁵N, ¹⁹F. ³¹P and ²⁹Si, and deuterated versions thereof. The second inlet port 114 may introduce parahydrogen gas into the reaction chamber. Although not shown, one or more additional inlet ports can be included in the reaction chamber 110 in order to introduce other liquids or gases into the reaction chamber. Alternatively, two or more components can be mixed in a separate container to form an intermediate solution and then introduced to the reaction chamber 110 through one of the inlet ports 112, 114.

In accordance with an aspect, the first outlet port 116 enables venting of gas from the reaction chamber 110. A continuous flow of parahydrogen gas can be connected to the inlet port 114, which is vented from outlet port 116. The outlet port 116 may include a valve incorporated therein or connected in series therewith in order to ensure that a desired pressure is maintained in the reaction chamber 110. In accordance with an aspect, the reaction chamber 110 is configured to withstand a gas pressure of up to 10 bars or more.

In accordance with an aspect, the line connected to the outlet port 116 can be connected to a gas trap 122 and/or a storage tank 124 such that the gas vented from the reaction chamber 110 is not vented to atmosphere in order to implement safety protocols. Although not shown explicitly, a compressor, valves, gauges, or other common air system components can be connected to the vented gas line in order to safely store the gas vented from the reaction chamber 110.

In accordance with an aspect, the second outlet port 118 enables a reaction mixture to be drawn out of the reaction chamber 110 after a reaction to generate the hyperpolarized MRI probe is complete. The second outlet port 118 may include a valve incorporated therein or connected in series therewith such that a vacuum can be formed in the separator 120 and/or the collection vessel 130 that causes the liquid to be drawn up a tube in the reaction chamber 110 and into the separator. The valve can be closed in order to create the vacuum while the reaction is taking place and then opened once the reaction is complete in order to draw the reaction mixture out of the reaction chamber 110.

The separator 120 is configured to receive a reaction mixture containing a perfluorocarbon hyperpolarization transfer SABRE catalyst, solvents, and a hyperpolarized MRI probe from the reaction chamber and separate the hyperpolarized MRI probe from the catalyst. In accordance with an aspect, the separator 120 may include a filter medium, which may include an adsorbent such as fluorous silica gel. The separator 120 can be configured to dilute the reaction mixture with water and/or ethanol prior to filtering the diluted mixture through the filter medium. The catalyst will be absorbed in the fluorous silica gel and separated from the hyperpolarized probe in the solvent. In accordance with another aspect, the filter medium can be omitted and the mixture can be separated by agitating the mixture and extracting the ethanol layer from the other layer containing a fluorous solvent-catalyst mixture. Optionally, the reaction mixture can be diluted with water prior to agitation and extraction such that a water/ethanol layer is extracted from the column. Alternatively, the methods above can be adapted when methanol is used as a solvent by adding a step for concentration and redissolution using water. Finally, the separated reaction mixture 132 including the hyperpolarized MRI probe is collected in a collection vessel 130, which can be, e.g., a vacuum flask or other device.

It will be appreciated that the hyperpolarized MRI probe, when extracted from the low magnetic field inside the reaction chamber 110, should be maintained in a high magnetic field to maintain polarization during separation, concentration, redissolution, and administration. The high magnetic field can be provided by one or more permanent magnets or electromagnets (e.g., coils) in proximity to the separator 120 and collection vessel 130. In some cases, the high magnetic field can be provided by a coil of an MRI machine located proximate the MRI probe infusion device. The strength of the high magnetic field may be determined based on the molecule being hyperpolarized.

Although not shown explicitly in FIG. 1, the reaction chamber 110 can include a number of internal components such as temperature control devices and/or a coil (e.g., solenoid) for generating an internal magnetic field within the reaction chamber 110, which are connected to an external controller via an interface 126. The controller is configured to execute software to control temperature inside the reaction chamber, to control flow of gas and liquid through the device, to monitor safety of the device and/or environment, to administer the desired quantity of the hyperpolarized probe to a patient, and/or to calculate a decay rate of the hyperpolarized probe as a function of the rate of flow of gas and/or liquid. The interface 126 may comprise electrical connections for providing control signals to the internal components and/or for receiving signals from one or more sensors (e.g., temperature sensors, gaussmeters, pressure gauges, etc.) connected to or located in the reaction chamber 110.

Some components of the device can be made of non-magnetic materials or plastics. For example, various connectors, sensors, valves, gauges, or the like can be made of brass or plastic in order to reduce any effects on the magnetic field induced in the reaction chamber.

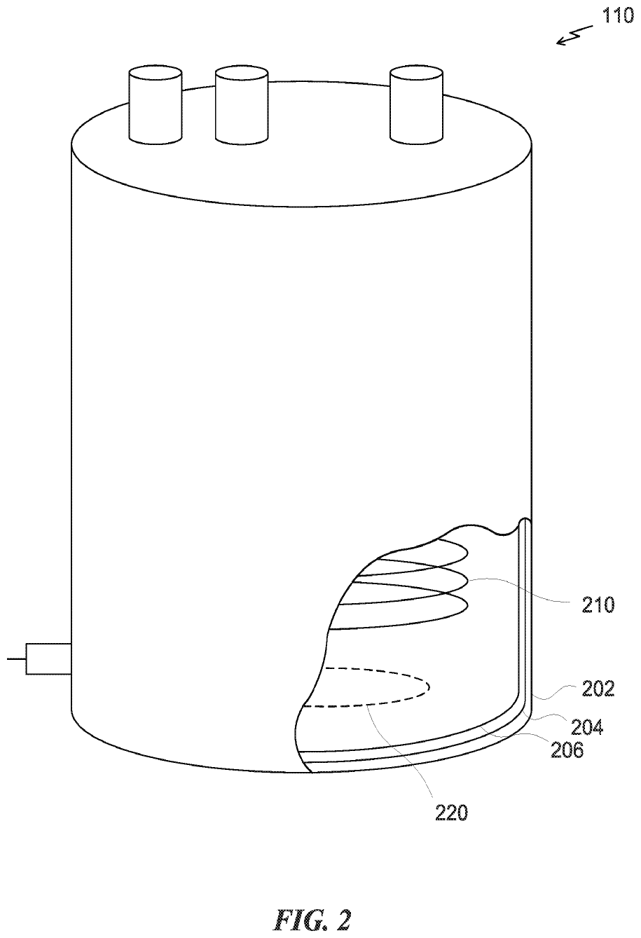

FIG. 2 illustrates the internal components of the reaction chamber 110, in accordance with an aspect of the disclosure. The reaction chamber 110 can comprise one or more layers 202, 204, 206 of metal shaped to form a chamber configured to contain liquid and/or gas at a volume and pressure suitable for performing the disclosed reactions therein. As shown in FIG. 2, the reaction chamber 110 can include three metal layers forming a substantially cylindrical shape. It will be appreciated that the reaction chamber is not limited to the cylindrical shape and that other desired shapes are contemplated to be used as a reaction chamber. In accordance with an aspect, the layers 202, 204, 206 comprise a ferromagnetic alloy. In accordance with some aspects, the one or more layers 202, 204, 206 may comprise a nickel-iron (Ni—Fe) alloy such as Invar, Permalloy, or Mu-Metal. At least one layer 202, 204, 206 is configured to shield the reaction chamber from a magnetic field associated with an external source, such as the Earth's magnetic field or other electrical components that may give off EM radiation. In accordance with an aspect, the layers 202, 204, 206 comprise a Mu-Metal shield that attenuates a magnetic field from the external source to have a strength of less than or equal to a threshold value in the one or more reaction chambers. In accordance with an aspect, the threshold value is 10 nT. In accordance with some aspects, different layers can comprise different materials. For example, an outer layer 202 may comprise a ferromagnetic alloy used to shield the interior of the chamber from external magnetic fields while an inner layer 206 may comprise a separate material that is unreactive with the solvents or chemicals introduced to the reaction chamber.

In accordance with an aspect, the reaction chamber 110 also includes a coil 210 used to generate an electromagnetic field within the reaction chamber in response to a current being passed therethrough. In accordance with some aspects, the coil is a high-homogeneity solenoid. The current can supply a constant or an alternating magnetic field of 0-200 milliTeslas (mT) and/or a radio frequency of 0 to 500 MHz.

In accordance with an aspect, the reaction chamber 110 also includes a temperature control device 220, which can include both a heating element and a cooling element. The temperature control device may include non-magnetic (i.e., nonferrous) material. In accordance with an aspect, the cooling element comprises a liquid nitrogen bath external to at least the inner layer 206 of the reaction chamber such that at least one surface of the layer 206 can be in contact with liquid nitrogen. Cooling is provided due to evaporation of liquid nitrogen as heat is drawn from the reaction chamber through the surface. The resulting nitrogen gas can be vented to the atmosphere and/or vented to the gas trap 122. The heating element can be a resistive heating element, such as a convection coil, that is proximate to the surface of the layer 206 or inserted into the reaction chamber through a port in the reaction chamber. In operation, a temperature controller can be connected to a temperature sensor/probe and configured to operate the heating and cooling elements to regulate a temperature of the reaction chamber 110. In accordance with an aspect, the temperature control device 220 is configured to maintain a temperature of the reaction chamber or reaction chambers between −25° C. to 100° C. In accordance with other aspects, the temperature of the reaction chamber(s) can be cycled between two or more different temperatures up to 10-20 times within a period of 1 to 5 minutes. For example, in operation, liquid nitrogen can be dispensed into the bath to cool the reaction chamber to a desired low temperature. Once the reaction chamber 110 has reached the low temperature and/or the liquid nitrogen has boiled off into nitrogen gas, the heating element can be turned on to heat the reaction chamber 110 to a desired high temperature. This cycle can be repeated a number of times during the reaction.

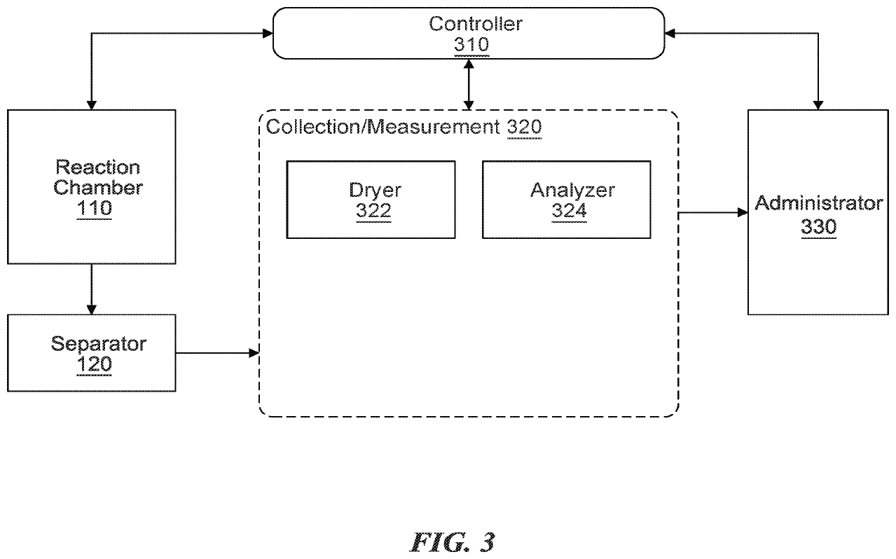

FIG. 3 schematically illustrates the components of the MRI probe infusion device, and their arrangement, in accordance with an aspect of the disclosure. The MRI probe infusion device includes at least one reaction chamber 110, a separator 120, collection and measurement equipment 320, and an MRI probe administrator device 330. The reaction chamber 110, collection and measurement equipment 320, and the MRI probe administrator device 330 are connected to a controller 310. The infusion device is also equipped with a dryer or concentrator 322 and an analyzer 332 for analyzing at least one of a purity or a concentration of the hyperpolarized MRI probe in a solution.

In accordance with an aspect, the controller 310 comprises software and/or hardware for operating the MRI probe infusion device. The controller 310 can include a computer device comprising at least one processor, a memory, and one or more input/output devices. Software, stored in the memory, can be executed by the processor to control the functions of the MRI probe infusion device. In accordance with an aspect, the controller 310 includes a processor configured to execute instructions that cause the processor to: control a flow of gas and/or liquid through the device, monitor a safety metric of the device and/or environment, administer a desired quantity of the hyperpolarized MRI probe to the patient, and/or calculate a decay rate of the hyperpolarized MRI probe as a function of a rate of flow of the gas and/or the liquid.

Although not shown explicitly, the controller 310 can receive inputs from one or more sensors that provide feedback in order to control the operation of the MRI probe infusion device. For example, the sensors can include a temperature sensor for monitoring the temperature of the reaction chamber and controlling the temperature control devices (i.e., the heating and/or cooling elements). The sensors can also include pressure transducers, flow meters, or other types of sensors to monitor the flow of liquid and/or gas flowing into or out of the reaction chamber 110. The sensors can also include a gas leak detector and/or an oxygen level monitor to maintain safety protocols when operating the device. For example, the gas leak detector can monitor the ambient environment around the device to monitor a level of hydrogen gas that could leak from the reaction chamber and/or any related gas lines or storage tanks. The oxygen level monitor can detect the level of oxygen in the room to prevent accidental asphyxiation due to leaking nitrogen gas. It will be appreciated that any other necessary sensors such as encoders, limit switches, proximity sensors, optical sensors, and the like can be included in the device to monitor one or more operating characteristics or the state of any component or actuator incorporated therein.

The controller 310 can also include an interface to enable human input through, e.g., a touchscreen display, keypad or keyboard, switches, buttons, or the like. This interface can allow a technician to operate the device manually. Alternatively, the interface can include a wired or wireless interface such that the controller can communicate with an external terminal (e.g., computer device, server device, tablet device, or the like) used to control the operation of the MRI probe infusion device. For example, the controller 310 can include a network interface that enables the device to connect to another terminal via a wired or wireless interface. The terminal can display a graphical user interface that enables the technician to interact with various menus in order to control operation of the device.

As shown in FIG. 3, in an aspect, the MRI probe infusion device includes collection and measurement equipment 320. In a clinical setting, the mixture containing the hyperpolarized MRI probe can be analyzed and further processed to facilitate automated generation of the hyperpolarized MRI probe and administration to a patient (e.g., human or animal) undergoing treatment. In accordance with an aspect, the collection and measurement equipment 320 can include a dryer 322 configured to concentrate the solution or emulsion containing the hyperpolarized MRI probe received from the one or more MRI probe separators 120 to form a concentrate. The dryer 322 can comprise a spray dryers and/or an evaporative dryer. In an aspect, the one or more evaporative dryers form one or more azeotropes with an inert solvent, resulting in a lower temperature for the evaporative dryers.

For example, a spray dryer can be implemented that comprises a container and a nozzle configured to vaporize the solvent such that the hyperpolarized MRI probe is deposited on the walls of the container. The solvent vapors can be vented to a gas trap and/or storage tank. The solid hyperpolarized MRI probe can be dissolved in a buffer solution (e.g., saline or a mixture of saline and ethanol) to form a solution containing a desired concentration of the hyperpolarized MRI probe. The collection and measurement equipment 320 can also include an analyzer 324 and/or other measurement tools used to analyze the reaction mixture prior to and/or after concentration and dilution in order to determine the efficacy of the reaction within the reaction chamber and/or adjust operating parameters in order to create a buffered solution at the desired concentration of the hyperpolarized MRI probe. For example, after analyzing the buffered solution, the controller 310 can use the measured concentration of the hyperpolarized MRI probe in the buffered solution in order to dispense additional buffer solution to reach a target concentration. In an aspect, the analyzer 324 can comprise a liquid chromatography/mass spectrometer (LC/MS) and/or a nuclear magnetic resonance (NMR) spectrometer.

The administrator 330 can include any equipment necessary for metering and delivery of the hyperpolarized MRI probe to a patient. The administrator 330 is configured to administer a dose of the solution containing the desired concentration of the hyperpolarized MRI probe to an animal or patient in need thereof. In accordance with an aspect, the administrator 330 may include, but is not limited to, peristaltic pumps, fluid lines, and the like. In an aspect, the administrator 330 can include a power injector common for other MRI or X-ray contrast agents. In another aspect, the administrator 330 can include an aerosol delivery attachment for pulmonary administration.

Again, it will be appreciated that the separator 120, collection and measurement equipment 320, and administrator 330 should be maintained in a high magnetic field during extraction and preparation of the hyperpolarized MRI probe prior to delivery to the patient. The magnetic field can be provided by one or more permanent or electromagnets, which may be included proximate the device and/or provided by a separate device such as an MRI machine.

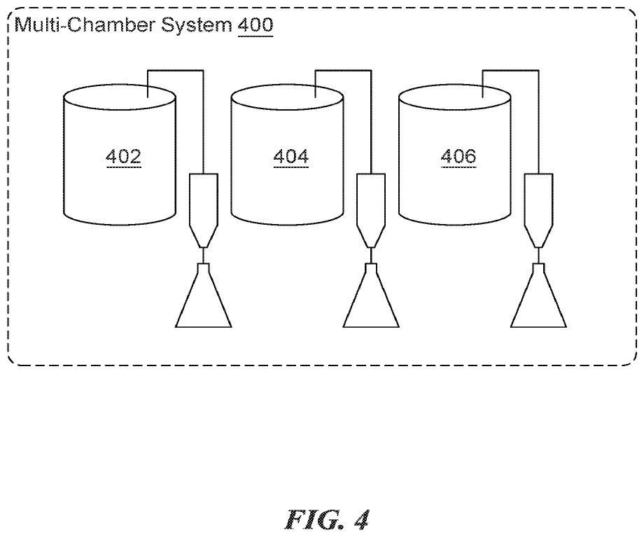

FIG. 4 illustrates an MRI infusion device equipped with multiple reaction chambers and the separation of the hyperpolarization probe from the reaction mixture from each reaction chamber, in accordance with an aspect of the disclosure. As shown in FIG. 4, the MRI infusion device 400 may include two or more reaction chambers 402, 404, 406 in order to facilitate multiple reactions for different hyperpolarized MRI probes substantially simultaneously and/or to increase a total volume of a single hyperpolarized MRI probe through multiple independent reactions performed in parallel. For example, if it takes 10 minutes to generate a single dose of the hyperpolarized MRI probe from a particular reactor, multiple reaction chambers can be used to create a separate dose every 5 or 3.3 minutes, for example, using a 2 or 3 chamber system, respectively. Each reaction chamber 402, 404, 406 can be associated with a separate and distinct separator 120, collection and measurement equipment 320, and/or administrator 330, as described above. Alternatively, multiple reaction chambers can share a single separator 120, collection and measurement equipment 320, and/or administrator 330.

It will be appreciated that, in some cases, the one or more reaction chambers are configured to be operable in series. For example, a reaction mixture created based on a reaction performed in a first reaction chamber can be provided to a second reaction chamber, where a second reaction can be performed. The reactions can be the same reaction or a different reaction (e.g., by introducing a different gas or chemical compound in the second reaction chamber, for example).

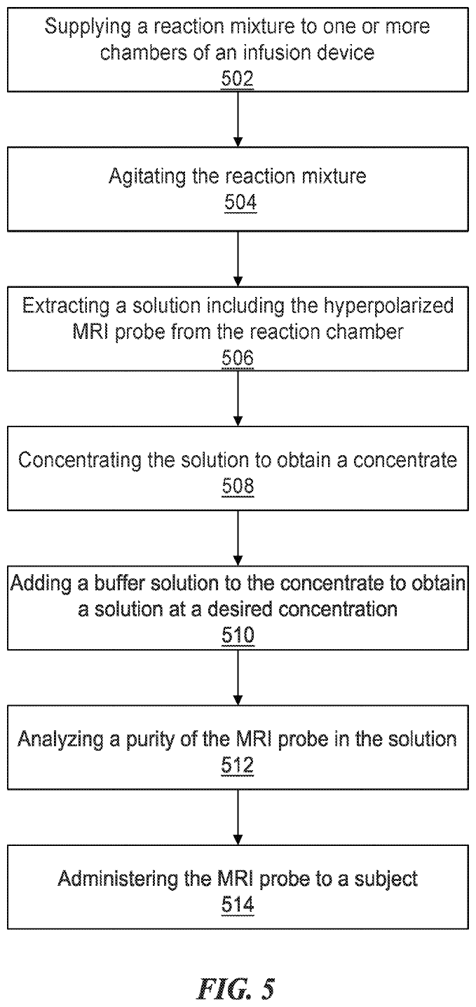

FIG. 5 illustrates some of the steps involved in the method of preparing a hyperpolarized MRI probe for administering to a patient, in accordance with an aspect of the disclosure. At step 502, a reaction mixture is supplied to one or more chambers of an MRI probe infusion device. Again, the reaction mixture can include a perfluorinated SABRE catalyst comprising a d-block element and a perfluorinated ligand, a solvent, a co-ligand, and a substrate to be hyperpolarized into an MRI probe.

In accordance with an aspect of the present disclosure, the substrate may be selected from one of 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

In accordance with an aspect of the present disclosure, the perfluorinated ligand is of Formula (I): [Lₘ-(NHC)—(Y—Z)_{q}] or a salt thereof. In the Formula (I), each L is independently selected from hydrogen, adamantyl, a substituted or unsubstituted aromatic, or a substituted or unsubstituted heteroaromatic group; NHC is a 4 to 7-membered N-heterocyclic carbenyl group where NHC is bound to the d-block element via a carbene; each Y is independently selected from a bond or a spacer group; each Z is a perfluorinated tag; m is an integer from 1 to 4; and q is an integer from 1 to 3. The perfluorinated tag is one of a perfluorinated C₃₋₆₀ group comprising only carbon and fluorine atoms; a perfluorinated C₃₋₄₀ group comprising only carbon and fluorine atoms; or a perfluorinated C₃₋₂₀ group.

In accordance with an aspect of the present disclosure, the perfluorinated ligand is selected from one of

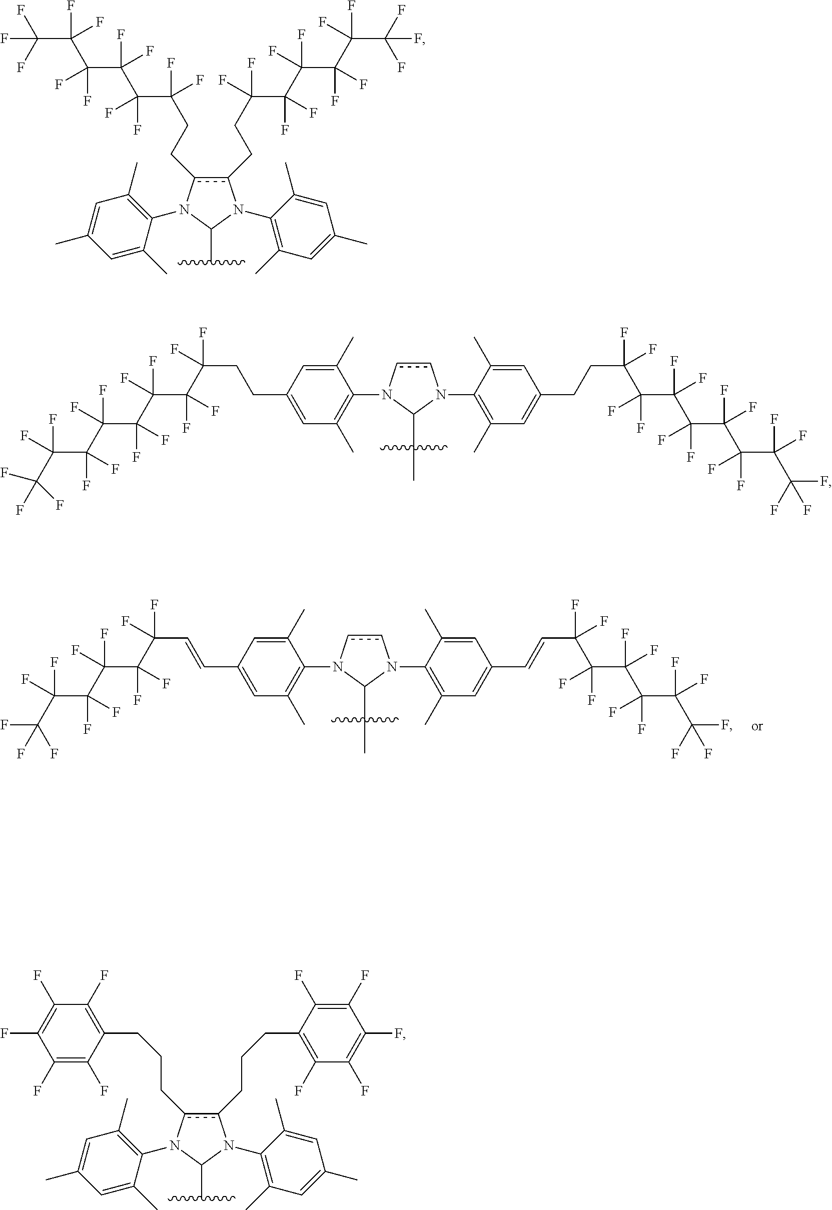

or a salt thereof, and wherein [symbol] is a single bond or a double bond, and [symbol] represents the bond to the d-block element via the carbene.

At 504, the reaction mixture is agitated via the injection of parahydrogen gas or a mixture of parahydrogen and nitrogen gas. For example, the inlet port 114 connected to the parahydrogen gas can be connected to a tube that descends into the bottom of the reaction chamber 110 such that gas entering the reaction chamber bubbles up through the solution containing the solvent, catalyst, and substrate. In accordance with an aspect, the coil 210 and/or the temperature control device 220 can be controlled to maintain a target temperature of the reaction chamber 110, or cycle the temperatures between two or more temperatures, in order to facilitate the reaction to generate the hyperpolarized MRI probe. In accordance with an aspect, a magnetic field is applied within the reaction chamber using the coil 210 that is suitable for hyperpolarization of the perfluorinated SABRE catalyst and the substrate to hyperpolarize the substrate into a hyperpolarized MRI probe.

At step 506, a solution including the hyperpolarized MRI probe is extracted from the reaction chamber. The solution may be separated into at least two parts by separating the hyperpolarized MRI probe from the reaction mixture by at least one of filtration, extraction, or column chromatography to obtain a solution containing the hyperpolarized MRI probe. In accordance with an aspect, the solution is filtered through the separator 120 to separate the catalyst from the hyperpolarized MRI probe dissolved in a solvent comprising ethanol, ethanol and water mixture, methanol, or ethanol and methanol mixture. In accordance with an aspect of the present disclosure, the solvent may be selected from one of a perfluorohexane/diethyl ether mixture, a methoxy nonafluorobutane and ethyl acetate mixture with a non-polar solvent, a perfluorohexane and ether mixture, a perfluorobutyl methyl ether and ethyl acetate mixture, an ether, a fluorocarbon derivative of THF FC 75, a decafluoromethoxy trifluoromethyl pentane, a hexafluoro propanol, a nonafluorobutyl methyl ether, a perfluoromethyl cyclohexane, a perfluoroalkane, a perfluorohexane, or a methoxy nonafluorobutane.

At step 508, the solution is concentrated to obtain a concentrate. The solution can be dried using one or more dryers to evaporate a solvent and increase the concentration of the hyperpolarized MRI probe in the solution. In some cases, all of the solvent are removed such that a powder, a solid residue, or a viscous liquid containing the hyperpolarized MRI probe remains on a surface of the dryer.

At step 510, a buffer solution is added to the concentrate to obtain a solution at a desired concentration. In accordance with an aspect, a saline or saline and ethanol solution can be added to the concentrate in order to create a buffered solution with a desired concentration of the hyperpolarized MRI probe.

At step 512, a purity and/or concentration of the hyperpolarized MRI probe in the buffered solution is analyzed. In some aspects, the analysis can be performed with at least one of a liquid chromatography/mass spectrometer or a NMR spectrometer. In accordance with some aspects, the results of the analysis can be used to further dilute the solution with additional buffer solution to adjust the concentration of the hyperpolarized MRI probe in the solution.

At step 514, the hyperpolarized MRI probe can be administered to a subject (e.g., a patient or animal). The hyperpolarized MRI probe can be administered using the administrator 330 in a desired dose and/or at a desired interval.

Again, it will be appreciated that step 504 is performed in a low magnetic field inside the reaction chamber, and steps 506-512 are performed in a high magnetic field (e.g., orders of magnitude larger than the low magnetic field inside the reaction chamber) provided by one or more permanent magnets and/or electromagnets external to the reaction chamber.

In an aspect, the hyperpolarized MRI probe can be separated from the reaction mixture in the MRI separator by a fluorophobic pass through a filter medium as illustrated in FIG. 6 and further described in Example 8.

FIG. 7 illustrates an aspect of the hyperpolarization, separation of the probe, and recovery of the SABRE catalyst.

In another aspect, the hyperpolarized MRI probe can be separated from the reaction mixture in the MRI separator by a fluorophobic pass, which is preceded by a fluorophilic pass, as illustrated in FIG. 8 and further described in Example 9.

In another aspect, FIG. 9 illustrates a process for separation of the probe and injection to the subject (e.g., animal or patient).

Aspects, including embodiments, of the present disclosure may be beneficial alone or in combination, with one or more other aspects or embodiments. Without limiting the foregoing description, certain non-limiting aspects of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) In an aspect (1), an MRI probe infusion device is provided comprising:
(i) one or more reaction chambers, each reaction chamber comprising:
   (a) a structure configured to attenuate a magnetic field within the reaction chamber from an external source, wherein a strength of the magnetic field within the reaction chamber from the external source is less than a threshold value;
   (b) one or more inlet ports;
   (c) a coil configured to generate an electro-magnetic field within the reaction chamber;
   (d) one or more temperature control devices; and
   (e) one or more outlet ports;
(ii) one or more MRI probe separators configured to receive a reaction mixture containing a perfluorinated SABRE catalyst, a solvent, and a hyperpolarized MRI probe from the one or more reaction chambers and extract the hyperpolarized MRI probe from the reaction mixture; and
(iii) one or more MRI probe collectors configured to form a solution containing a desired concentration of the hyperpolarized MRI probe.

(2) In an aspect (2), the structure (a) is a mu-metal shield that attenuates a magnetic field from the external source to have a strength of less than or equal to 10 nT in the one or more reaction chambers.

(3) In an aspect (3), the one or more inlet ports include one or more gas ports and one or more liquid ports.

(4) In an aspect (4), each reaction chamber is configured to withstand a gas pressure of at least 10 bars.

(5) In an aspect (5), the magnetic field within the reaction chamber induced by the coil is between 0-200 milliTeslas.

(6) In an aspect (6), the one or more temperature control devices comprise a non-magnetic heating element and/or cooling element configured to provide a temperature cycling of the reaction chamber.

(7) In an aspect (7), the one or more temperature control devices provides cooling with nitrogen gas from evaporation of liquid nitrogen, and heating by a convection coil.

(8) In an aspect (8), the one or more temperature control devices are configured to provide a temperature cycling or maintain a temperature of each reaction chamber between −25° C. to 100° C.

(9) In an aspect (9), the one or more reaction chambers are equipped to perform hyperpolarization with a reaction mixture containing the perfluorinated SABRE catalyst, the solvent, and a substrate to be hyperpolarized into the hyperpolarized MRI probe.

(10) In an aspect (10), the solvent is a one phase system or a two phase system comprising water, methanol, ethanol, a fluorous solvent, or a mixture thereof.

(11) In an aspect (11), the one or more MRI probe separators are configured to separate the hyperpolarized MRI probe from the perfluorinated SABRE catalyst by one of filtration; extraction; or column chromatography.

(12) In an aspect (12), the MRI probe infusion device further includes a gas trap, a gas leak detector, and/or an oxygen level monitor.

(13) In an aspect (13), the MRI probe infusion device further includes a processor configured to execute instructions that cause the processor to: control a flow of gas and/or liquid through the device, monitor a safety metric of the device and/or environment, administer a desired quantity of the hyperpolarized MRI probe to the patient, or calculate a decay rate of the hyperpolarized MRI probe as a function of a rate of flow of the gas and/or the liquid.

(14) In an aspect (14), the one or more MRI probe concentrators include one or more dryers.

(15) In an aspect (15), the one or more dryers comprise evaporative dryers that form one or more azeotropes with an inert solvent, resulting in a lower temperature for the evaporative dryers.

(16) In an aspect (16), the one or more reaction chambers include at least two reaction chambers configured to be operable in series or in parallel.

(17) In an aspect (17), components of the MRI probe infusion device are made of non-magnetic materials or plastics.

(18) In an aspect (18), a method of administering a hyperpolarized MRI probe to a patient in need thereof is provided, the method including:
(i) providing an MRI probe infusion device,
(ii) supplying to one or more of the reaction chambers a reaction mixture comprising a perfluorinated SABRE catalyst comprising a d-block element and a perfluorinated ligand, a solvent, a co-ligand, and a substrate to be hyperpolarized into an MRI probe,
(iii) agitating the reaction mixture, wherein the agitation is provided via bubbling parahydrogen gas or a mixture of parahydrogen and nitrogen gas through the reaction mixture,
(iv) applying a magnetic field suitable for hyperpolarization of the perfluorinated SABRE catalyst and the substrate to hyperpolarize the substrate into a hyperpolarized MRI probe,
(v) separating the hyperpolarized MRI probe from the reaction mixture by at least one of filtration, extraction, or column chromatography to obtain a solution containing the hyperpolarized MRI probe,
(vi) concentrating the hyperpolarized MRI probe present in the solution obtained in step (v) to obtain a concentrate and reconstituting the concentrate into a solution of desired concentration of the hyperpolarized MRI probe for administering to the patient;
(vii) analyzing at least one of a purity or a concentration of the hyperpolarized MRI probe present in the solution; and
(viii) administering the hyperpolarized MRI probe to the patient.

(19) In an aspect (19), the solvent comprises water, methanol, ethanol, a fluorous solvent, or a mixture thereof.

(20) In an aspect (20), the solvent is selected from a perfluorohexane/diethyl ether mixture, a methoxy nonafluorobutane and ethyl acetate mixture with a non-polar solvent, a perfluorohexane and ether mixture, a perfluorobutyl methyl ether and ethyl acetate mixture, an ether, a fluorocarbon derivative of THF FC 75, a decafluoromethoxy trifluoromethyl pentane, a hexafluoro propanol, a nonafluorobutyl methyl ether, a perfluoromethyl cyclohexane, a perfluoroalkane, a perfluorohexane, and a methoxy nonafluorobutane.

(21) In an aspect (21), the substrate comprises ¹H, ¹³C, ¹⁵N, ¹⁹F, ³¹P, ²⁹Si, or a combination thereof.

(22) In an aspect (22), the substrate further comprises ²D.

(23) In an aspect (23), the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-N₂-hydrazide), ¹³C₂,¹⁵N₃-metronidazole, ¹⁵N₂-1-aminoisoquinoline (1-AIQ), deuterated versions thereof, and salts thereof.

(24) In an aspect (24), the substrate is of Formula (II):

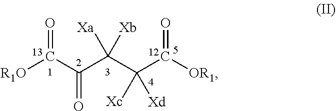

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and wherein Xa, Xb, Xc, and Xd are each independently hydrogen or deuterium, provided that at least one of Xa, Xb, Xc, and Xd is deuterium, or a pharmaceutically acceptable salt thereof.

(25) In an aspect (25), the perfluorinated ligand is of Formula (I): [Lₘ-(NHC)—(Y—Z)_{q}] or a salt thereof, and wherein: each L is independently selected from hydrogen, adamantyl, a substituted or unsubstituted aromatic, or a substituted or unsubstituted heteroaromatic group, NHC is a 4 to 7-membered N-heterocyclic carbenyl group where NHC is bound to the d-block element via a carbene, each Y is independently selected from a bond or a spacer group, each Z is a perfluorinated tag, m is an integer from 1 to 4, and q is an integer from 1 to 3.

(26) In an aspect (26), NHC is a 5-membered N-heterocyclic carbenyl group.

(27) In an aspect (27), the 5-membered N-heterocyclic carbenyl group is imidazole-based, imidazoline-based, or thiazole-based.

(28) In an aspect (28), NHC is a 4,5-disubstituted, a 1,3-disubstituted, or a 1,3,4,5-tetrasubstituted imidazole-based or imidazoline-based 5-membered N-heterocyclic carbenyl group.

(29) In an aspect (29), NHC is a 4,5-disubstituted imidazolidinyl, a 1,3-disubstituted imidazolidinyl, a 1,3,4,5-tetrasubstituted imidazolidinyl, a 4,5-disubstituted 2,3-dihydro-imidazolyl, a 1,3-disubstituted 2,3-dihydro-imidazolyl, or a 1,3,4,5-tetrasubstituted 2,3-dihydro-imidazolyl.

(30) In an aspect (30), the perfluorinated ligand is of Formula (Ia) or (Ib):

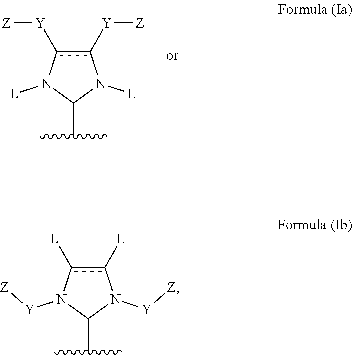

or a salt thereof, and wherein: each L independently is hydrogen, adamantyl, a substituted or unsubstituted aromatic, or a substituted or unsubstituted heteroaromatic group, each Y independently is a bond or a spacer group, each Z independently is a perfluorinated tag, [symbol] is a single bond or a double bond, and [symbol] represents the bond to the d-block element via the carbine.

(31) In an aspect (31), the perfluorinated ligand is of Formula (Ic) or (Id):

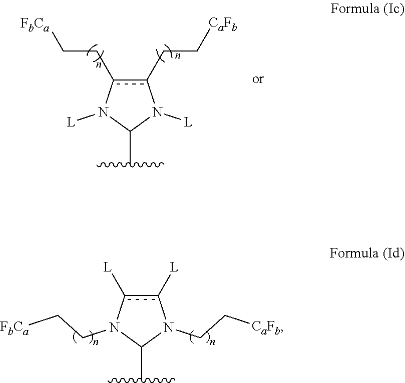

or a salt thereof, and wherein: each L independently is hydrogen, adamantyl, a substituted or unsubstituted aromatic, or a substituted or unsubstituted heteroaromatic group, a is 4 to 20, b=2a+1 or b=a−1, each n independently is an integer from 0 to 4, [symbol] is a single bond or a double bond, and [symbol] represents the bond to the d-block element via the carbine.

(32) In an aspect (32), the perfluorinated ligand is of Formula (Ie) or (If):

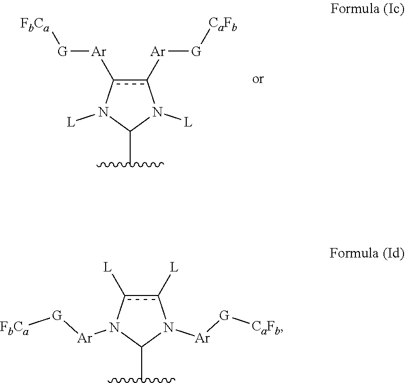

or a salt thereof, and wherein: each L independently is hydrogen, adamantyl, a substituted or unsubstituted aromatic, or a substituted or unsubstituted heteroaromatic group, each Ar independently is a substituted or unsubstituted aromatic, or a substituted or unsubstituted heteroaromatic group, each G independently is a bond C₁₋₆ alkyl, C₁₋₆ alkenyl, or C₁₋₆ heteroalkyl, a is 4 to 20, b=2a+1 or b=a−1, [symbol] is a single bond or a double bond, and [symbol] represents the bond to the d-block element via the carbine.

(33) In an aspect (33), each L independently is hydrogen, adamantyl, 2-methylphenyl, 3-methylphenyl, 4-methylphenyl, 2,4-dimethylphenyl, 2,5-dimethylphenyl, 2,6-dimethylphenyl, 3,5-dimethylphenyl, 2,4,6-trimethylphenyl, 2-ethylphenyl, 3-ethylphenyl, 4-ethylphenyl, 2,4-diethylphenyl, 2,5-diethylphenyl, 2,6-diethylphenyl, 3,5-diethylphenyl, 2,4,6-triethylphenyl, 2-npropylphenyl, 3-npropylphenyl, 4-npropylphenyl, 2,4-di-npropylphenyl, 2,5-di-npropylphenyl, 2,6-di-npropylphenyl, 3,5-di-npropylphenyl, 2,4,6-tri-npropylphenyl, 2-isopropylphenyl, 3-isopropylphenyl, 4-isopropylphenyl, 2,4-di-isopropylphenyl, 2,5-di-isopropylphenyl, 2,6-di-isopropylphenyl, 3,5-di-isopropylphenyl, 2,4,6-tri-isopropylphenyl, 2-isobutylphenyl, 3-isobutylphenyl, 4-isobutylphenyl, 2,4-di-isobutylphenyl, 2,5-di-isobutylphenyl, 2,6-di-isobutylphenyl, 3,5-di-isobutylphenyl, 2,4,6-tri-isobutylphenyl, 2-secbutylphenyl, 3-secbutylphenyl, 4-secbutylphenyl, 2,4-di-secbutylphenyl, 2,5-di-secbutylphenyl, 2,6-di-secbutylphenyl, 3,5-di-secbutylphenyl, 2,4,6-tri-secbutylphenyl, 2-tbutylphenyl, 3-tbutylphenyl, 4-tbutylphenyl, 2,4-di-tbutylphenyl, 2,5-di-tbutylphenyl, 2,6-di-tbutylphenyl, 3,5-di-tbutylphenyl, 2,4,6-tri-tbutylphenyl, 2-cyclohexylphenyl, 3-cyclohexylphenyl, 4-cyclohexylphenyl, 2,4-di-cyclohexylphenyl, 2,5-di-cyclohexylphenyl, 2,6-di-cyclohexylphenyl, 3,5-di-cyclohexylphenyl, or 2,4,6-tri-cyclohexylphenyl.

(34) In an aspect (34), each L independently is hydrogen or 2,4,6-trimethylphenyl.

(35) In an aspect (35), each Y independently is a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group.

(36) In an aspect (36), the perfluorinated tag is a perfluorinated C₃₋₆₀ group comprising only carbon and fluorine atoms.

(37) In an aspect (37), the perfluorinated tag is a perfluorinated C₃₋₄₀ group comprising only carbon and fluorine atoms.

(38) In an aspect (38), the perfluorinated tag is a perfluorinated C₃₋₂₀ group.

(39) In an aspect (39), the perfluorinated tag is selected from a C₄F₉ group, a C₅F₁₁ group, a C₆F₁₃ group, a C₇F₁₅ group, a C₈F₁₇ group, a C₉F₁₉ group, a C₁₀F₂₁ group, a C₆F₅ group, C₄F₇ group, a C₅F₉ group, a C₆F₁₁ group, a C₇F₁₃ group, a C₈F₁₅ group, a C₉F₁₇ group, and a C₁₀F₁₉ group.

(40) In an aspect (40), the perfluorinated ligand is one of:

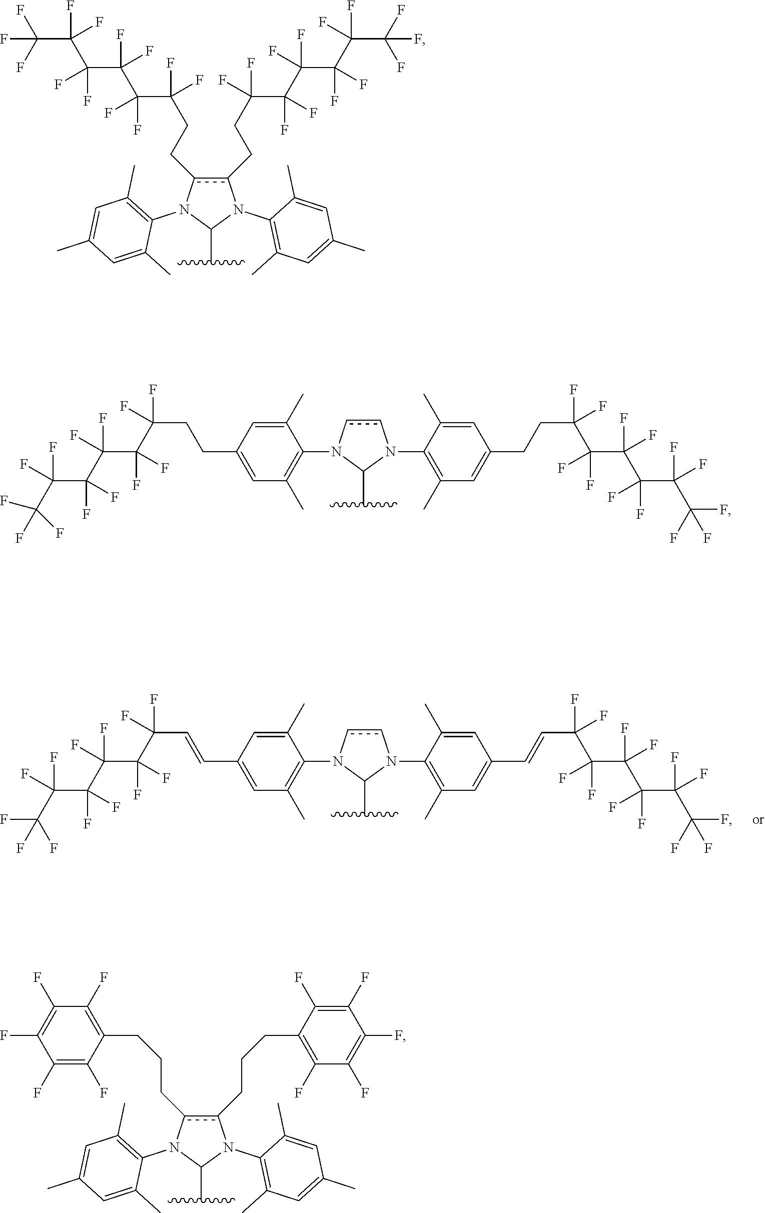

or a salt thereof, and wherein: [symbol] is a single bond or a double bond, and [symbol] represents the bond to the d-block element via the carbene.

(41) In an aspect (41), the d-block element is a transition metal.

(42) In an aspect (42), the d-block element is one of Co, Rh, Ir, Ru, Pd, Pt, or Mt.

(43) In an aspect (43), the method further includes diagnosing stages of a disease or monitoring treatment progress of the patient having the disease using the hyperpolarized MRI probe.

(44) In an aspect (44), the disease is cancer or an adverse vascular condition.

(45) In an aspect (45), the cancer is selected from breast cancer, colon cancer, rectal cancer, bladder cancer, endometrial cancer, kidney cancer, lung cancer, melanoma, non-Hodgkin lymphoma, pancreatic cancer, prostate cancer, and thyroid cancer.

(46) In an aspect (46), the adverse vascular condition is selected from myocardial infarction, stroke, and pulmonary disease.

(47) In an aspect (47), the pulmonary disease is selected from COPD, lung fibrosis, long-term COVID-19 symptom, or a combination thereof.

The following examples further illustrate various aspects of the present disclosure but, of course, should not be construed as in any way limiting its scope.

### Example 1

This example illustrates a method of preparing 1H,1H,2H,2H-perfluorooctyl-N,N′-bis(2,4,6-trimethylphenyl)-9,10-diamine:

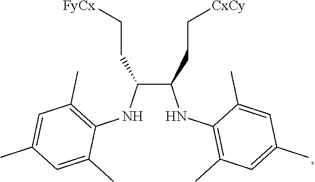

wherein x=6 and y=13.

A 1.7 M solution of tert-butyl lithium in pentane (5 mL, 9 mmol, 8 equiv.) was added to a solution of 1H,1H,2H,2H-perfluorooctyl iodide (2 g, 4 mmol, 4 equiv.) in dry Et₂O (60 mL) at −78° C. After the mixture had been stirred for 20 min at −78° C., the solid N,N′-dimesitylethanediimine (0.30 g, 1.03 mmol, 1 equiv.) was added portion wise. The reaction mixture was stirred for 4 h. The reaction was slowly warmed to −30° C. and quenched with a saturated solution of ammonium chloride (0.6 mL).

Water (20 mL) was added, the organic layer was separated, and the aqueous layer was extracted with diethyl ether (3×15 mL). The combined organic layers were dried over anhydrous MgSO₄ and concentrated. The crude product was purified by flash column chromatography (4:1 hexane/dichloromethane (DCM)) to yield diamine, diimine, and threo-diamine (0.6 g, 60% yield, white solid). ¹H NMR (400 MHz, CDCl₃) δ 1.64-1.82 (m, 2H, CH₂CHHCH(NHAr)), 2.00 (s, 12H, o-CH₃), 2.07-2.27 (m, 4H, CHHCHHCH(NHAr)), 2.33 (s, 6H, p-CH₃), 2.67-2.91 (m, 2H, CHHCH₂CH(NHAr)), 2.95 (br s, 2H, NH), 3.23 (br d, ³J_{H-H}=10 Hz, 2H, CH), 6.82 (s, 4H, Ar—CH) ppm. ¹⁹F NMR (282.23 MHz, CDCl₃) δ −83.8 (t, ³J_{F-F}=10 Hz, 6F, CF₃), −114.4 (m, 4F, CF₂CH₂), −121.9 (m, 4F, CF₂CF₂CH₂), −122.8 (m, 4F, CF₃CF₂CF₂CF₂), −123.4 (m, 4F, CF₃CF₂CF₂), −126.1 (m, 4F, CF₃CF₂) ppm. ¹³C NMR (400 MHz, CDCl₃) δ 18.3 (o-CH₃), 20.3 (p-CH₃), 21.7 (m, CF₂CH₂CH₂), 29.2 (t, ²J_{F-C}=22.2 Hz, CF₂CH₂), 57.1 (CH), 107-115 (m, 8 CF₂), 117.4 (qt, ¹J_{F-C}=288.3 Hz, ²J_{F-C}=33.3 Hz, CF₃), 118.7 (tt, ¹J_{F-C}=255.1 Hz, ²J_{F-C}=31.0 Hz, CF₂CH₂), 128.6 (Ar—C), 129.9 (Ar—CH), 131.4 (Ar—C), 140.4 (Ar—C) ppm. MS (ESI), m/z (%): 990 [M+H]⁺ (100).

### Example 2

This example illustrates the synthesis of trans-4,5-bis(3,3,4,4,5,5,6,6,7,7,8,8,8-tridecafluorooctyl)-1,3-bis(2,4,6-trimethylphenyl)-4,5-dihydroimidazolium tetrafluoroborate:

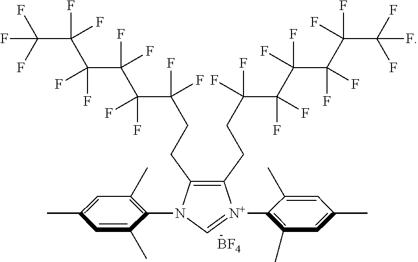

A mixture of 1H,1H,2H,2H-Perfluorooctyl-N,N′-bis(2,4,6-trimethylphenyl)-9,10-diamine (0.250 g, 0.25 mmol) from Example 1, ammonium tetrafluoroborate (˜10% molar excess) (0.030 g, 0.25 mmol), and triethyl orthoformate (0.5 mL) was heated to 125° C. and stirred for 15 h. After cooling to room temperature, the solution was evaporated and the solid was triturated with diethyl ether (6×3 mL). The residue was redissolved in acetone, filtered, and concentrated to yield to the dihydroimidazolium tetrafluoroborate. (0.2 g, 70% yield, yellowish solid). ¹H NMR (400 MHz, acetone-d₆) δ 2.15-2.70 (m, 8H, CF₂CH₂CH₂), 2.34 (s, 6H, p-CH₃), 2.51 (s, 6H, o-CH₃), 2.94 (br s, 2H, NH), 5.16 (m, 2H, CH₂CH₂CH), 7.17 (s, 4H, Ar—CH), 9.06 (s, 1H, N—CH═N) ppm. ¹⁹F NMR (400 MHz, acetone-d₆) δ −81.6 (t, ⁴J_{F-F}=10 Hz, 6F, CF₃), −115.1 (m, 4F, CF₂CF₂CH₂), −122.5 (m, 4F, CF₂CF₂CH₂), −123.6 (m, 4F, CF₃CF₂CF₂CF₂), −124.5 (m, 4F, CF₃CF₂CF₂), −126.9 (m, 4F, CF₃CF₂CF₂), −150.9 (4F, BF₄) ppm. ¹³C NMR (400 MHz, acetone-d₆) δ 17.4 (o-CH₃), δ 17.7 (o-CH₃), 19.9 (p-CH₃), 24.4 (m, CF₂CH₂CH₂), 26.2 (t, ²J_{F-C}=22.2 Hz, CF₂CH₂), 67.9 (CH), 105-118 (m, 8 CF₂), 118 (qt, ¹J_{F-C}=288.3 Hz, ²J_{F-C}=33.3 Hz, CF₃), 118.9 (tt, ¹J_{F-C}=254 Hz, ²J_{F-C}=31.0 Hz, CF₂CH₂), 129.5 (Ar—C), 130.1 (Ar—CH), 130.5 (Ar—CH), 135.8 (Ar—C), 136 (Ar—C), 140.8 (Ar—C) ppm, 159.7 (N—C═N) ppm. MS (ESI) m/z (%): 999 [M+H]⁺ (100).

### Example 3

This example illustrates the synthesis of trans-4,5-Bis(3,3,4,4,5,5,6,6,7,7,8,8,8-tridecafluorooctyl)-1,3bis(2,4,6-trimethylphenyl)-4,5-dihydroimidazolium chloride:

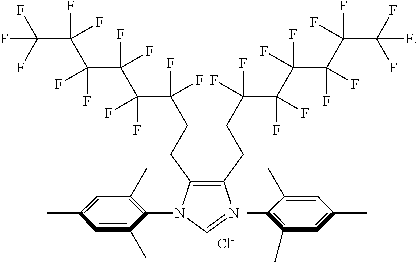

Dihydroimidazolidium tetrafluoroborate salt (0.5 g, 0.46 mmol) was dissolved in MeOH (1.5 mL) and passed through a short column of ion exchange resin Amberlite 400. The column was washed with MeOH until no spot was visible via TLC under UV. The solvent was removed, and the resulting yellowish solid was dried with a vacuum pump to yield product (0.48 g, 99%). ¹H NMR (400 MHz, acetone-d₆) δ 2.15-2.70 (m, 8H, CF₂CH₂CH₂), 2.34 (s, 6H, p-CH₃), 2.51 (s, 6H, o-CH₃), 2.94 (br s, 2H, NH), 5.16 (m, 2H, CH₂CH₂CH), 7.17 (s, 4H, Ar—CH), 9.06 (s, 1H, N—CH═N) ppm. ¹⁹F NMR (400 MHz, acetone-d₆) δ −81.8 (t, ⁴J_{F-F}=10 Hz, 6F, CF₃), −115.1 (m, 4F, CF₂CF₂CH₂), −122.5 (m, 4F, CF₂CF₂CH₂), −123.6 (m, 4F, CF₃CF₂CF₂CF₂), −124.5 (m, 4F, CF₃CF₂CF₂), −127 (m, 4F, CF₃CF₂CF₂) ppm. ¹³C NMR (400 MHz, acetone-d₆) δ 17.4 (o-CH₃), δ 17.7 (o-CH₃), 19.9 (p-CH₃), 24.4 (m, CF₂CH₂CH₂), 26.2 (t, ²J_{F-C}=22.2 Hz, CF₂CH₂), 67.9 (CH), 105-118 (m, 8 CF₂), 118 (qt, ¹J_{F-C}=288.3 Hz, ²J_{F-C}=33.3 Hz, CF₃), 118.9 (tt, ¹J_{F-C}=254 Hz, ²J_{F-C}=31.0 Hz, CF₂CH₂), 129.5 (Ar—C), 130.1 (Ar—CH), 130.5 (Ar—CH), 135.8 (Ar—C), 136 (Ar—C), 140.8 (Ar—C) ppm, 159.7 (N—C═N) ppm. MS (ESI) m/z (%): 999 [M+H]⁺ (100).

### Example 4

This example illustrates a method of synthesis of a fluorinated SABRE catalyst containing a transition metal in accordance with an aspect of the invention.

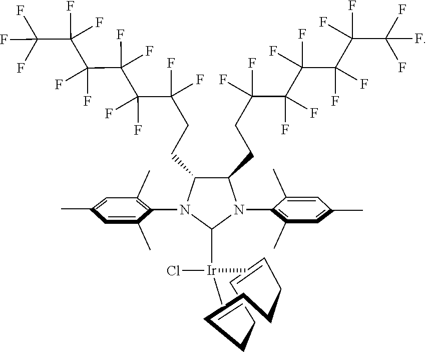

Potassium tert-butoxide (112 mg, 1.00 mmol, 2.5 eq.) was added to a stirred solution of trans-4,5-bis(3,3,4,4,5,5,6,6,7,7,8,8,8-tridecafluorooctyl)-1,3-bis(2,4,6-trimethylphenyl)-4,5-dihydroimidazolium chloride (320 mg, 0.88 mmol, 2.2 eq.) from Example 3 in tetrahydrofuran (10 mL) at room temperature in a glove box. The resulting suspension was stirred for 30 minutes. A solution of [Ir(COD)Cl]₂ (268 mg, 0.40 mmol, 1.0 eq.) was added and the resulting solution was stirred at room temperature overnight (Cowley et al., J. Am. Chem. Soc., 133, 6134-6137 (2011)). The solvent was removed under reduced pressure to give the crude product and dried overnight under vacuum. This sample was dissolved in hexane and added to a 60 mL filter packed with SiO₂ gel in hexane. The crude solution was absorbed on the top of the silica gel, then hexane was added, to elute the compound, to give 104 mg (40% yield). ¹H NMR (400 MHz, CDCl₃) δ 1.04, 1.18, 1.49, 1.76, 2.13, 2.22, 2.25, 2.4, 2.48, 2.66, 2.94, 3.73, 4.01, 4.2, 6.84, 6.91 6.97 ppm. ¹⁹F NMR (400 MHz, CDCl₃) δ−81.08, −114.33, −122.05, −123.07, −124.02, −126.39 ppm. ¹³C NMR (100 MHz, CDCl₃) δ 18.7, 20.95, 21.07, 21.34, 26.45, 27.21, 29.83, 30.74, 31.42, 49.93, 55.12, 68.11, 68.47, 83.26, 86.69, 108.32, 111.0, 112.95, 115.85, 117.75, 118.69, 129.02, 129.3, 130.36, 130.55, 134.71, 134.8, 135.32, 136.33, 137.92, 138.28, 138.45, 138.5, 206.72 ppm. MS (ESI) m/z (%): 1299 [M+H]⁺ (100).

### Example 5

This example illustrates a method of hyperpolarizing a [1-¹³C]pyruvate in accordance with an aspect of the disclosure, as shown in Scheme 1.

Scheme 1. Hyperpolarization of [1-¹³C]Pyruvate with Phenyl Trifluoromethyl Sulfoxide as Co-Ligand.

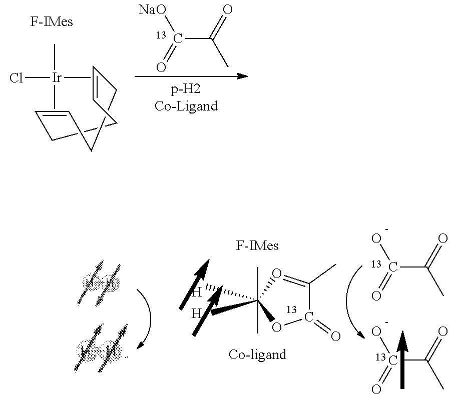

wherein co-ligand=phenyl trifluoromethyl sulfoxide.

In the reaction scheme above, hyperpolarization of [1-¹³C]pyruvate was performed using SABRE in SHield Enabled Alignment Transfer to Heteronuclei (SABRE-SHEATH) (Theis et al., J. Am. Chem. Soc., 137, 1404-1407 (2015) and Truong et al., J. Phys. Chem. C, 119, 8786-8797 (2015)) tailored for the 13C nucleus (Barskiy et al., ChemPhysChem, 18, 1493-1498 (2017)) using the co-ligand approach developed by Duckett and co-workers (Iali et al., Angew. Chemie—Int. Ed., 58, 10271-10275 (2019)). Sodium [1-¹³C]-pyruvate and deuterated methanol-d₄ solvent were purchased from Sigma-Aldrich and used without any further purification. The [IrCl(COD)(F-IMes)] SABRE catalyst used for this Example was prepared according to Example 4. The active catalyst used herein was prepared with a fixed ratio of substrate to Ir(F-IMes) SABRE catalyst of Example 4, and phenyl trifluoromethyl sulfoxide (PTFSO) in 0.6 mL of methanol-d₄ in a 5 mm NMR tube.

Parahydrogen was generated using a Gas-Delivery Manifold. Ultra-high-purity hydrogen gas (Airgas) was fed into a ParaHydrogen flow cryostat (Xeus technology LTD) and enriched to about 50% parahydrogen in the presence of a spin-exchange catalyst (Fe₂O₃) at liquid nitrogen temperature (77K). The p-H₂ flow was directed via PTFE tubing to a mass flow controller (MFC, Sierra Instruments SmartTrak 100 series) set at 90 sec/m and directed to a conventional 5 mm NMR tube (Norell) to allow bubbling through the sample. The entire pH₂ line was pressurized to 100 psi.

The magnetic shield condition was as follows. Magnetic fields near or below ˜1 μT were achieved with an apparatus consisting of a solenoid coil placed inside a mu-metal shield (Magnetic Shield Corporation, model No. ZG-206). The shield was degaussed using internal homebuilt coils driven by a Variac when necessary. The solenoid had a 41 mm diameter (40 mm core, 20 cm long windings with 220 turns AWG20 (0.9 mm) Cu wire and with 220Ω resistor in series. The solenoid coil was driven by commercial 1.5V batteries with a variable-resistance decade box in series to provide finer control of the internal magnetic field inside the shield. Typical values of the field within the shield were between ±1.2 μT, with SABRE SHEATH experiments typically between −0.7 μT and +0.8 μT in the sample region. The values were monitored between SABRE experiments using a Lakeshore Cryotronics Gaussmeter (Model No. 475 DSP with HMMA-2512-VR Hall Probe).

MR experiments were performed using a 1 T Magritek Spinsolve benchtop NMR spectrometer. All ¹³C NMR spectra were taken with ¹H decoupling turned off throughout the duration of the experiment. Time required to manually transfer the sample from the shield region to the magnet for low-field NMR acquisition was usually <5 s.

The efficient hyperpolarization transfer from nascent p-H₂-derived hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(PTFSO)₂(F-IMes)], [1-¹³C]pyruvate) and p-H₂ in deuterated methanol. FIG. 6 depicts, in the top curve, a single-scan HP ¹³C spectrum obtained for the hyperpolarized probe. The bottom curve shows a single-scan thermally polarized ¹³C signal from 4 M sodium [1-¹³C]acetate using similar acquisition parameters. Enhancement is ε-9000 and polarization is about P(¹³C)˜1%.

All experiments were performed with the solution containing fluorinated catalyst, co-ligand (phenyl trifluoromethyl sulfoxide) and [1-¹³C]Pyruvate in 0.6 mL CD₃OD. The ratio and concentration can be further adjusted to attain better enhancement and polarization. The results were obtained for 8 mM fluorinated catalyst, 16 mM phenyl trifluoromethyl sulfoxide (PTFSO) and 30 mM [1-¹³C]Pyruvate in 0.6 mL CD₃OD. The experiments were performed at room temperature, ˜100 scc/m p-H₂ flow rate and 96 PSI p-H₂ overpressure. The parahydrogen used in this example came from a low-cost 50% p-H₂ generator. Each experiment, the p-H₂ bubbling was applied for ˜1 min, the sample was quickly transferred to the 1 T NMR spectrometer for detection and the sample was then returned to the mu-metal shield to continue p-H₂ bubbling for the next experiment. The ¹³C signal enhancement was computed by comparing HP signal area-undercurve (AUC) to external ¹³C signal thermal signal reference (4M sodium [1-¹³C]acetate) using Eq. 1:

$$ε {(}^{13} C ) = \frac{{S}_{HP}}{{S}_{REF}} \cdot \frac{{C}_{REF}}{{C}_{HP}} \cdot \frac{{A}_{REF}}{{A}_{HP}} , \qquad ( 1 )$$

where S_{HP} and S_{REF} are ¹³C signals from HP [1-¹³C]pyruvate and thermal signal reference [1-¹³C]acetate, C_{REF} and C_{HP} are concentrations of thermal signal reference [1-¹³C]acetate (4 M) and of HP [1-¹³C]pyruvate, respectively, and A_{REF} and A_{HP} are effective cross-sections of the NMR tubes for the thermal signal reference [1-¹³C]acetate and HP [1-¹³C]pyruvate samples.

### Example 6

This example illustrates a method of hyperpolarizing a [1-¹³C]pyruvate in accordance with an aspect of the invention, as shown in Scheme 2.

Scheme 2. Hyperpolarization of [1-¹³C]Pyruvate Using the Perfluorinated SABRE Catalyst of Example 4 with Dimethyl Sulfoxide as Co-Ligand.

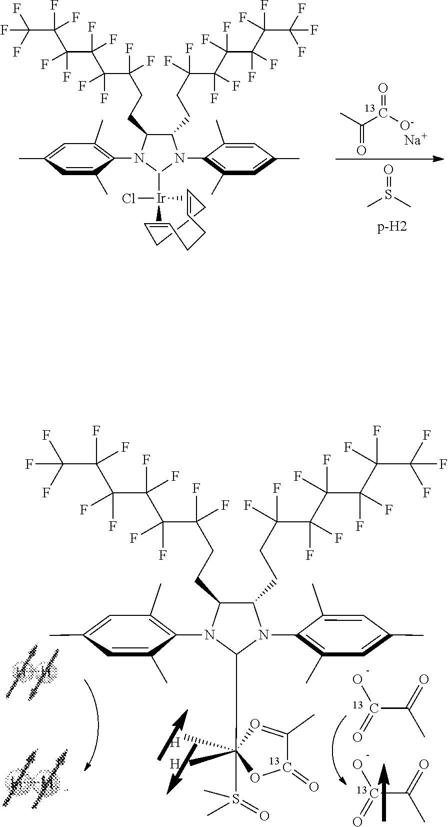

In the reaction scheme above, hyperpolarization of [1-¹³C]pyruvate was performed using SABRE in SHield Enables Alignment Transfer to Heteronuclei (SABRE-SHEATH) (Theis et al., J. Am. Chem. Soc., 137, 1404-1407 (2015) and Truong et al., J. Phys. Chem. C, 119, 8786-8797 (2015)) tailored for the ¹³C nucleus (Barskiy et al., ChemPhysChem, 18, 1493-1498 (2017)) using the co-ligand approach developed by Duckett and co-workers (Iali et al., Angew. Chemie—Int. Ed., 58, 10271-10275 (2019)). Sodium [1-¹³C]-pyruvate and deuterated methanol-d₄ solvent were purchased from Sigma-Aldrich and used without any further purification. The [IrCl(COD)(F-IMes)] SABRE catalyst used for this Example was prepared according to Example 4. The active catalyst used herein was prepared with a fixed ratio of substrate [1-¹³C]pyruvate, Ir(F-IMes) SABRE catalyst of Example 4, and co-ligand dimethyl sulfoxide (DMSO), in 0.6 mL of methanol-d₄ in a 5 mm NMR tube.

Parahydrogen enriched to about 70 to 95% was used and directed via PTFE tubing to a mass flow controller (MFC, Sierra Instruments SmartTrak 100 series) set between 50 to 120 scc/m into a medium wall 5 mm NMR tube (Norell) to allow bubbling through the sample. The entire pH₂ line was pressurized values between 50 and 110 psi.

The polarization transfer magnetic field was established as follows. Magnetic fields near or below ˜1 μT were achieved with an apparatus consisting of a solenoid coil placed inside a three-layered mu-metal shield (6 in. ID & 15 in. in length, part number ZG-206, Magnetic Shield Corp., Bensenville, IL). The magnetic field was created using a custom-built solenoid coil and a triple independent channel DC power supply (KEITHLEY 2231A-30-3). The solenoid had a 41 mm diameter (40 mm core, 20 cm long windings with 220 turns AWG20 (0.9 mm) Cu wire and with 220Ω resistor in series. The solenoid coil was driven with a variable-resistance decade box in series to provide finer control of the internal magnetic field inside the shield. Typical values of the field within the shield were between ±1.2 μT, with SABRE SHEATH experiments typically between −0.7 μT and +0.8 μT in the sample region.

MR experiments were performed using an 80 MHz Magritek Spinsolve benchtop NMR spectrometer. The following acquisition parameters were used:
spectra width (SW)=5 kHz;
dwell time (DT)=150 μs;
number of scans (ns)=1,
receiver gain=16;
excitation pulse angle (a)=90°;
¹³C resonance frequency=20.25232790 MHz.

All ¹³C NMR spectra were taken with ¹H decoupling turned off throughout the duration of the experiment. Time required to manually transfer the sample from the shield region to the magnet for low-field NMR acquisition was usually <5 s.

The hyperpolarization transfer from p-H₂-derived iridium hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate, co-ligand (dimethyl sulfoxide) and p-H₂ in 0.5 mL deuterated or non-deuterated methanol.

The ¹³C signal enhancement was computed by comparing HP signal area-undercurve (AUC) to external ¹³C signal thermal signal reference (4M sodium [1-¹³C]acetate) using Eq.:

$$ε {(}^{13} C ) = \frac{{S}_{HP}}{{S}_{REF}} \cdot \frac{{C}_{REF}}{{C}_{HP}} \cdot \frac{{A}_{REF}}{{A}_{HP}} , \qquad ( 1 )$$

where S_{HP} and S_{REF} are ¹³C signals from HP [1-¹³C]pyruvate and thermal signal reference [1-¹³C]acetate, C_{REF} and C_{HP} are concentrations of thermal signal reference [1-¹³C]acetate (4 M) and of HP [1-¹³C]pyruvate, respectively, and A_{REF} and A_{HP} are effective cross-sections of the NMR tubes for the thermal signal reference [1-¹³C]acetate and HP [1-¹³C]pyruvate samples. The percentage of ¹³C polarization (% P¹³_{C}) was computed by multiplying the signal enhancement (ε¹³_{C}) by thermal ¹³C nuclear spin polarization at 1.81 T (1.5681*10⁻⁴%) in accordance with Equation S2: (% P¹³_{C}), =ε¹³_{C}*1.56181*10⁻⁶*100.

The fluorinated SABRE catalyst activation took less than 15 minutes, with the ¹³C polarization percentage shown in FIG. 7, and is performed by bubbling ˜95% p-H₂ at a flow rate of 90 standard cubic centimeters per minute (scc/m) at 8 atm p-H₂ partial pressure, which leads to the formation of Complex 2, Complex 3a, Complex 3b, and pyruvate, as depicted in FIG. 8, in accord with the notation introduced by Duckett and co-workers (Iali et al., Angew. Chemie—Int. Ed., 58, 10271-10275 (2019)). Without wishing to be bound by any particular theory, it is believed that Complex 3B is the primary SABRE-active species.

### Example 7

This example demonstrates the effects on hyperpolarization of [1-¹³C]pyruvate, exhibited by changes in parahydrogen pressure and flow rate, as well as the effect of magnetic transfer field, temperature, and concentration of the fluorinated catalyst and DMSO. In addition, the relaxation dynamics of the [1-¹³C]pyruvate were also studied.

Hyperpolarization of [1-¹³C]pyruvate was repeated using SABRE in SHield Enables Alignment Transfer to Heteronuclei (SABRE-SHEATH), as described in Example 6 above, and the effects of parahydrogen pressure and flow rate, as well as the effect of magnetic transfer field, temperature, and concentration of the fluorinated catalyst and DMSO, were studied.

p-H₂ parameters such as the pressure and flow rate were evaluated and the polarization percentage results are set forth in FIGS. 9A and 9B. The NMR samples contained in 30 mM sodium [1-¹³C]pyruvate, 2.6 mM fluorinated SABRE catalyst, and 40 mM dimethyl sulfoxide (DMSO), the mixing field was at 0.4 μT and temperature at 0° C. As is apparent from the results set forth in FIGS. 9A and 9B, as the parahydrogen flow rate and pressure increased, the polarization percentage also increased.

The temperature and magnetic field in the micro Tesla regime were evaluated and the 13C polarization level and polarization transfer magnetic field at 0° C. are set forth in FIGS. 10A and 10B, respectively. The NMR samples contained in 30 mM sodium [1-¹³C]pyruvate, 2.6 mM fluorinated SABRE catalyst, and 40 mM dimethyl sulfoxide (DMSO), p-H₂ flow and pressure 70 scc/m and 100 PSI. As is apparent from the results set forth in FIGS. 10A and 10B, the best polarization transfer occurs at temperatures between −20° C. and 5° C. and a mixing field between 0.3 μT and 0.5 μT. The optimum temperature is −7.24° C. and the optimum mixing field is 0.4 μT.

The perfluorinated SABRE catalyst and DMSO concentrations were evaluated at a temperature of 0° C., a magnetic transfer field of 0.4 μT, a p-H₂ flow of 90 scc/m, and a p-H₂ pressure of 110 PSI, and the polarization percentages are set forth in FIGS. 11A and 11B. As is apparent from the results set forth in FIG. 11A, the polarization percentage increases as the perfluorinated SABRE catalyst concentration increases. However, the polarization percentage remains relatively consistent at concentrations above 20 mM.

As is apparent from the results set forth in FIGS. 12A and 12B, the relaxation dynamics of [1-¹³C]-pyruvate show that the total P_{13C} (bound+free) build-up time (T_{b}=6.6±3.0 s) is substantially shorter than the corresponding T₁ value of 16.1±0.9 s, which allows to reach P_{13C} levels up to 13.48%. In addition, relaxation dynamics at earth field and 1.8 T are about the same as a non-fluorinated SABRE catalyst T₁=28.9±1.6 s and 66.5±7 s, respectively.

The simultaneous exchange of p-H₂ and [1-¹³C]pyruvate on activated Ir(F-IMes) catalyst leads to buildup of ¹³C hyperpolarization. In that respect, FIG. 13 shows a representative spectrum of ¹³C-hyperpolarized [1-¹³C]-pyruvate with signal enhancement ε of ˜86500 fold, corresponding to P_{13C} of ˜13.48% obtained via comparison of the NMR signal intensity with a reference sample. The NMR samples contained in 20 mM sodium [1-¹³C]pyruvate, 2.6 mM fluorinated SABRE catalyst, and 40 mM dimethyl sulfoxide (DMSO), the mixing field was at 0.4 μT and temperature at 0° C. with a parahydrogen pressure and flow at 110 PSI and 90 scc/m, respectively.

Temperature has a profound effect on the exchange rates of [1-¹³C]pyruvate on Complex 3b of FIG. 7. In recent work, Adelabu et al. (ChemPhysChem, 23, e202100839 (2022)) showed that the monotonic disappearance of free HP resonance at low temperatures happened due to the slow exchange rate of Complex 3b into the free state. At room temperature (e.g., 22° C.), the exchange of [1-¹³C]pyruvate with the polarization transfer complex was faster, leading to hyperpolarization of both free and bound 3b species in the expected pyruvate:pre-catalyst ratio. In order to rapidly release the HP pyruvate from 3b, the HP solution was rapidly warmed up then the sample was inserted in the NMR detector (TomHon et al., J. Am. Chem. Soc., 144(1) 282-287 (2022)). FIG. 14 shows a variable temperature SABRE-SHEATH experiment using the saturated perfluorinated SABRE catalyst of Example 4. The NMR samples (in deuterated methanol) contained in 25 mM sodium [1-¹³C]pyruvate, 6 mM perfluorinated SABRE catalyst, and 47 mM dimethyl sulfoxide (DMSO), wherein the mixing field was at 0.4 μT, and the parahydrogen pressure and flow rate were set at 110 PSI and 90 scc/m, respectively.

As demonstrated by FIG. 14, the exchange rate of Complex 3b into the free state is faster even at low temperature, such as −10° C., where most of the HP [1-¹³C]pyruvate is in a free state. By decreasing the temperature of ¹³C SABRE-SHEATH to 0° C., P_{13C} maximum is achieved. The exchange remains fast enough to build-up preferentially the “free” HP [1-¹³C]pyruvate over Complex 3b.

### Example 8

This example illustrates an exemplary method for isolating hyperpolarized sodium [1-¹³C]pyruvate, which includes extraction and filtration.

Hyperpolarization of sodium [1-¹³C]pyruvate was performed using SABRE in SHield Enables Alignment Transfer to Heteronuclei (SABRE-SHEATH) tailored for the ¹³C nucleus using dimethyl sulfoxide (DMSO) as a co-ligand, [IrCl(COD)(F-IMes)] SABRE catalyst, and parahydrogen enriched to about 70 to 95% in deuterated methanol-d₄ solvent, as described in Examples 6 and 7. The SABRE samples were prepared in 0.5 mL CD₃OD, using 30 mM sodium [1-¹³C]pyruvate, 2.6 mM perfluorinated SABRE catalyst of Example 4, and 35 mM dimethyl sulfoxide (DMSO). The parahydrogen flow rate was established at 90 scc/m and pressurized to 8 bars, the mixing field was 0.4 μT, and the temperature was 0° C.

After the hyperpolarization procedure was completed, the sample was rapidly removed from the 0.40 μT field, depressurized, and 20% in volume (125 μL) of D₂O was added to the solution to precipitate the perfluorinated SABRE catalyst. The resulting mixture was transferred into a 1 mL plastic syringe mounted to a Luer-locked filter (Waters Oasis Prime HLB Plus Light Cartridge (Part Number: 186008866), and the aqueous solution was guided through the filter into a 5 mm NMR tube, already located into the adjacent 1.8 T benchtop NMR spectrometer. The whole procedure took about 1.15 to 1.30 minutes and no HP ¹³C signal was observed in any sample. The relaxation study presented above indicates that [1-¹³C]-T₁ relaxation time of pyruvate at earth field was substantially shorter (e.g., [1-¹³C]-T₁=28.9±1.6 s at Earth's field). Automation and faster solution transfer should allow the observation of HP [1-¹³C]-pyruvate signal in aqueous solutions.

### Example 9

This example illustrates an exemplary method for isolating hyperpolarized sodium [1-¹³C]pyruvate, which includes extraction by precipitation with organic solvent.

Precipitation and redissolution of HP [1-13C]pyruvate, which takes place in the same NMR tube where hyperpolarization, was performed (Schmidt et al., ACS Sensors, 7(11), 3430-3439 (2022)). The SABRE samples were prepared in CD₃OD, using between 20 and 30 mM sodium [1-¹³C]pyruvate, 7.5 mM fluorinated SABRE catalyst, and 50 mM dimethyl sulfoxide (DMSO), as described in Examples 6 and 7. 100 μL of this solution was transferred into 5 mm NMR tube and exposed to the SABRE-SHEATH hyperpolarization conditions with the same set-up and optimum conditions described in Examples 6 and 7. The solution was located inside a 3-layer mu-metal of 3″ I.D. and 9″ depth to shield external magnetic fields, combined with a custom-made solenoid to generate a static magnetic field B₀ of 0.4 μT. The NMR tubes were pressurized (110 psi, i.e., approximately 8 bar total pressure) with p-H₂ bubbling through the solution at a flow of 90 scc/m to activate the catalyst and to ¹³C-hyperpolarize the sodium [1-¹³C]pyruvate solution. Activation of the catalyst took place for 15 min at ambient temperature and magnetic field. For polarization build-up, the sample was placed in the static magnetic field (typically about 0.4 μT) and a water bath to regulate the reaction temperature at 0° C. After polarization, the NMR tube was rapidly transferred inside the NMR spectrometer at 1.8 T and kept at room temperature. The precipitation of pyruvate is performed after depressurization by adding 400 μL of ethyl acetate (EtOAc) to the HP solution and redissolved by adding 300 μL D₂O to reconstitute the pyruvate in water.

The NMR spectrum was acquired immediately after reconstitution in water using a 1.8 T benchtop NMR, and the results are set forth in the top spectrum of FIG. 15. In addition, the bottom spectrum of FIG. 15 shows a single-scan thermally polarized ¹³C signal from 4 M sodium [1-¹³C]acetate using similar acquisition parameters.

In addition, the ¹³C-pyruvate concentrations were determined by LCMS using a calibration curve for naturally occurring isotopic pyruvate. The pyruvate aqueous samples were further analyzed by ICP-MS (inductively coupled plasma Multi-Element Scan) for Iridium elemental content after the reconstitution SABRE-SHEATH methodology. The Iridium content was determined to be only about 150 ppb to about 300 ppm.

### Example 10

This example illustrates a method of the synthesis of 2,2,2-trifluoro-N-(4-iodo-2,6-dimethylphenyl)acetamide:

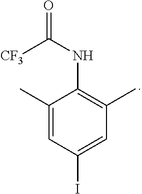

To a stirring solution of 2,6-diisopropylaniline (4.92 mL, 40.00 mmol, 1.0 equiv) in diethyl ether (50 mL) were added iodine (11.17 g, 44.00 mmol, 1.1 equiv) and a saturated sodium bicarbonate solution (30 mL). The solution was stirred at room temperature for 3 hours and gas evolution was observed. Excess iodine was destroyed by addition of sodium thiosulfate (1.33 g, 8.40 mmol). The phases were separated, and the aqueous phase was further extracted with diethyl ether (2×20 mL). The combined organic phases were washed with water (200 mL) and saturated solution of sodium thiosulfate. The organic phase was evaporated to dryness in vacuo to yield the product as a brown oil which slowly became solid under vacuum.

Hexane was added to dissolve product and the solution is filtered with Celite and evaporated and dried under vacuum. No further purification was necessary. ¹H NMR (400 MHz, CDCl₃): δ=7.14 (s, 2H), 4.45 (s, 2H), 2.04 (s, 6H).

### Example 11

This example illustrates a method of synthesis of N-(2,6-dimethyl-4-(3,3,4,4,5,5,6,6,7,7,8,8,9,9,10,10,10-tridecafluorooct-1-en-1-yl)phenyl)-2,2,2-trifluoroacetamide:

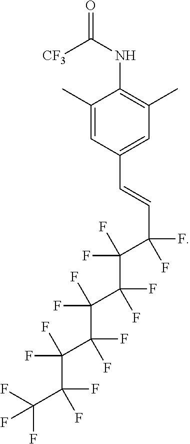 [SMILES: Cc1cc(/C=C/C(F)(F)C(F)(F)C(F)(F)C(F)(F)C(F)(F)C(F)(F)C(F)(F)C(F)(F)F)cc(C)c1NC(=O)C(F)(F)F]

In a glove box, palladium(II) acetate (0.16 g, 0.051 equiv, 0.71 mmol), sodium acetate (1.72 g, 1.50 equiv, 21.0 mmol), tricyclohexylphosphane (660 mg, 0.168 equiv, 2.35 mmol), tetrabutylammonium bromide (30 mg, 0.0067 equiv, 93 μmol) and 2,2,2-trifluoro-N-(4-iodo-2,6-dimethylphenyl)acetamide (4 g, 0.8 equiv, 0.01 mol) were added to a Schlenk flask with 10 mL of dimethyl formamide. The Schlenk flask was warmed to 90° C., then 3,3,4,4,5,5,6,6,7,7,8,8,9,9,10,10,10-tridecafluorooct-1-ene (4.84 g, 1 equiv, 14.0 mmol) was added to the reaction mixture and further heated to 120° C. The solution was stirred for 19 hours. After cooling, the mixture was filtered through Celite and washed with diethyl ether (50 mL). Water (50 mL) and diethyl ether (30 mL) were added, the organic phase was separated, and aqueous phase was extracted with Et₂O (3×15 mL). The combined organic layers were dried over anhydrous MgSO₄ and concentrated under reduced pressure to afford the crude product. Purification by column chromatography (hexane/DCM 4:1) gave fluoroamide N-(2,6-dimethyl-4-(3,3,4,4,5,5,6,6,7,7,8,8,9,9,10,10,10-tridecafluorooct-1-en-1-yl)phenyl)-2,2,2-trifluoroacetamide, which was further crystallized (5 g, 70%, white needles).

### Example 12

This example illustrates a synthesis of N-(2,6-dimethyl-4-(3,3,4,4,5,5,6,6,7,7,8,8,9,9,10,10,10-tridecafluorooctyl)phenyl)-2,2,2-trifluoroacetamide:

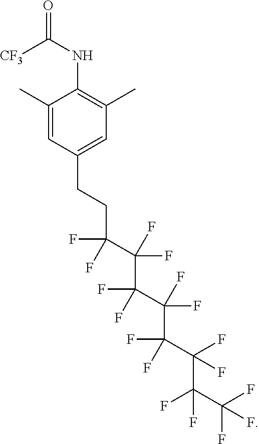 [SMILES: Cc1cc(CCC(F)(F)C(F)(F)C(F)(F)C(F)(F)C(F)(F)C(F)(F)C(F)(F)C(F)(F)F)cc(C)c1NC(=O)C(F)(F)F]

To a solution of N-(2,6-dimethyl-4-(3,3,4,4,5,5,6,6,7,7,8,8,9,9,10,10,10-tridecafluorooct-1-en-1-yl)phenyl)-2,2,2-trifluoroacetamide (0.40 g, 0.70 mmol) in ethyl acetate (10 mL) in a glass autoclave, 10% Pd/C (0.08 g, 0.07 mmol) was added. The autoclave was evacuated, filled with hydrogen gas to 500 kPa and stirred for 5 hours at room temperature. The mixture was filtered through Celite and washed with EtOAc (30 mL). The solvent was removed on vacuum rotary evaporator to afford N-(2,6-dimethyl-4-(3,3,4,4,5,5,6,6,7,7,8,8,9,9,10,10,10-tridecafluorooctyl)phenyl)-2,2,2-trifluoroacetamide.

### Example 13

This example illustrates a synthesis of 2,6-dimethyl-4-(3,3,4,4,5,5,6,6,7,7,8,8,9,9,10,10,10-tridecafluorooctyl)aniline:

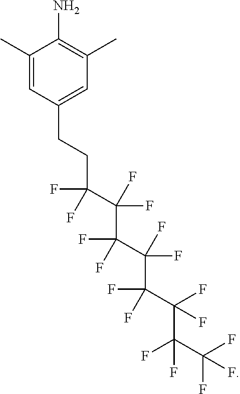 [SMILES: Cc1cc(CCC(F)(F)C(F)(F)C(F)(F)C(F)(F)C(F)(F)C(F)(F)C(F)(F)C(F)(F)F)cc(C)c1N]

To a solution of N-(2,6-dimethyl-4-(3,3,4,4,5,5,6,6,7,7,8,8,9,9,10,10,10-tridecafluorooctyl)phenyl)-2,2,2-trifluoroacetamide (0.14 g, 250 gmol) in n-butanol (1.5 mL), sodium hydroxide (0.10 g, 250 mmol) was added and the reaction mixture was heated to 110° C. for 21 hours. After cooling to room temperature, water (4 mL) and ethyl acetate (4 mL) were added and the organic phase was separated, washed with 1M solution of HCl (4 mL), saturated solution of NaHCO₃ (4 mL), and brine (4 mL). The aqueous phase was neutralized and extracted with ethyl acetate (3 times×4 mL). The organic layers were combined and dried over anhydrous magnesium sulfate and concentrated under reduced pressure to afford 2,6-dimethyl-4-(3,3,4,4,5,5,6,6,7,7,8,8,9,9,10,10,10-tridecafluorooctyl)aniline (0.50 g, 92%, brown crystals).

### Example 14

This example illustrates a synthesis of N,N′-Bis[2,6-dimethyl-4-(3,3,4,4,5,5,6,6,7,7,8,8,9,9,10,10,10-tridecafluorooctyl)phenyl]ethane-1,2-diimine:

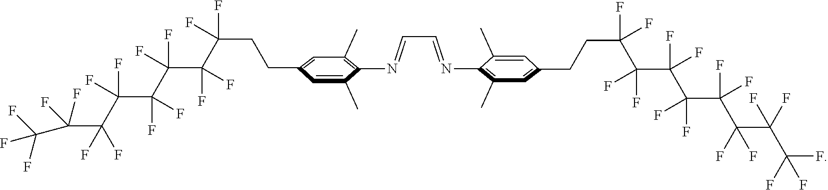

To the solution of 2,6-dimethyl-4-(3,3,4,4,5,5,6,6,7,7,8,8,9,9,10,10,10-tridecafluorooctyl)aniline (0.5 g, 1 mmol) in ethanol (5 mL), 40% aqueous solution of glyoxal (0.1 mL, 1 mmol) and catalytic amount of formic acid (few drops) were added. The reaction mixture was stirred for 20 hours at room temperature, during which yellow precipitate was formed. The solid was filtered and washed with cold ethanol (3×5 mL) to give clean N,N-bis(2,6-dimethyl-4-(3,3,4,4,5,5,6,6,7,7,8,8,9,9,10,10,10-tridecafluorooctyl)phenyl)ethane-1,2-diimine (0.2 g, 20%, yellow powder).

### Example 15

This example illustrates a synthesis of 1,3-bis(2,6-dimethyl-4-(3,3,4,4,5,5,6,6,7,7,8,8,9,9,10,10,10-tridecafluorooctyl)phenyl)-1H-imidazol-3-ium-2-ide, chloride salt:

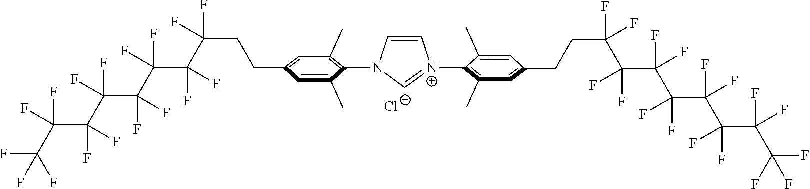

A flask was charged with N,N-bis(2,6-dimethyl-4-(3,3,4,4,5,5,6,6,7,7,8,8,9,10,10,10-tridecafluorooctyl)phenyl)ethane-1,2-diimine (2.40 g, 2.08 mmol) and tetrahydrofuran (80 mL). The mixture was cooled to 0° C. and suspension of paraformaldehyde (262 mg, 2.91 mmol) and conc. HCl (112 mg, 3.12 mmol) in dioxane (0.78 mL) was slowly added. The mixture was heated to reflux overnight. Purification was carried out by column chromatography. ¹H NMR (400 MHz, CDCl₃) δ 2.07, 2.55, 2.92, 7.37, 8.28, 9.65. MS (ESI) m/z (%): 1168.8 [M-TFA]¹ (100).

### Example 16

This example illustrates a synthesis of a fluorinated SABRE catalyst in accordance with an aspect of the invention.

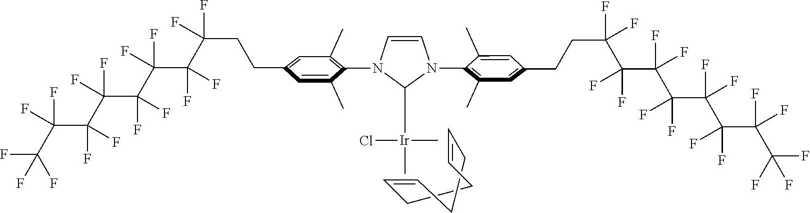

Potassium tert-butoxide (2.5 eq.) was added to a stirred solution of 1,3-bis(2,6-dimethyl-4-(3,3,4,4,5,5,6,6,7,7,8,8,9,9,10,10,10-tridecafluorooctyl)phenyl)-1H-imidazol-3-ium-2-ide, chloride (2.2 equiv) in tetrahydrofuran at room temperature in a glove box. The resulting suspension was stirred for 30 min. A solution of [Ir(COD)Cl]₂ (1.0 eq.) was added and the resulting solution was stirred at room temperature for 2 hours. The solvent was removed under reduced pressure to give the crude product and dried overnight under vacuum. This sample was purified with flash chromatography DCM/hexane (4:1) to obtain the fluorinated SABRE catalyst. MS (ESI) m/z (%): 1469 [M-Cl]⁺ (100).

### Example 17

This example illustrates the hyperpolarization of sodium pyruvate using the SABRE catalyst in accordance with an aspect of the invention, as shown in Scheme 3.

Scheme 3. Hyperpolarization of [1-¹³C]Pyruvate Using the Perfluorinated SABRE Catalyst of Example 16 with Dimethyl Sulfoxide as Co-Ligand.

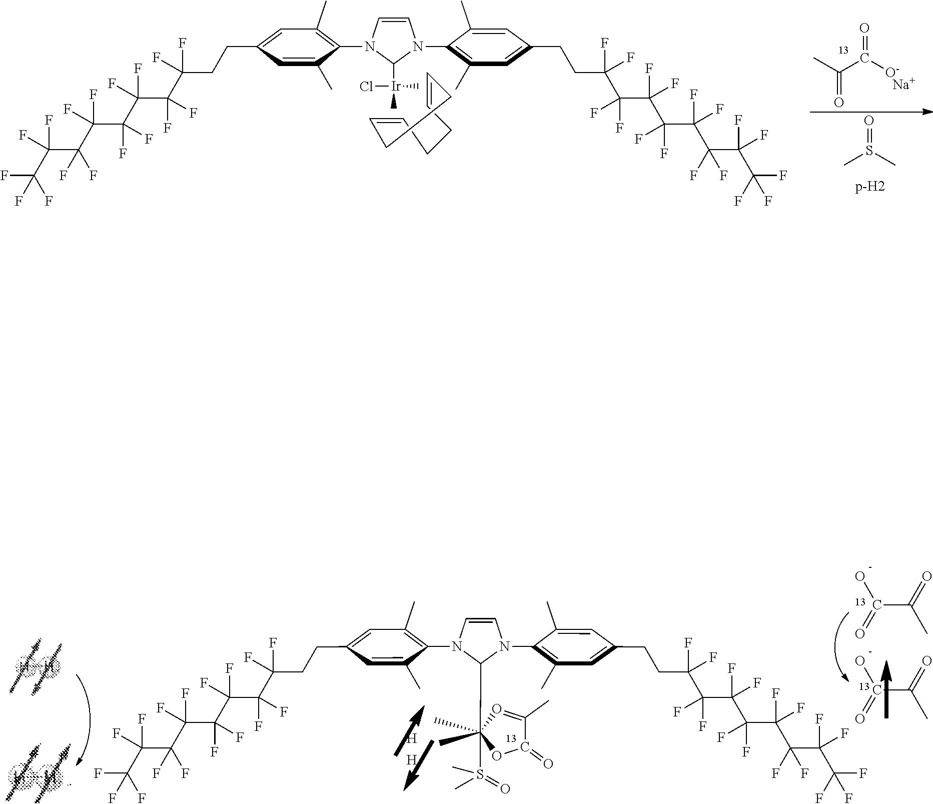

In the reaction scheme above, the efficient hyperpolarization transfer from p-H₂-derived hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate) and p-H₂ in deuterated methanol.

The SABRE samples were prepared in CD₃OD, using 40 mM sodium [1-¹³C]pyruvate, 6.6 mM perfluorinated SABRE catalyst of Example 16, and 50 mM dimethyl sulfoxide (DMSO), as described in Examples 6 and 7. The SABRE samples were exposed to the SABRE-SHEATH hyperpolarization conditions with the same set-up and optimum conditions described in Examples 6 and 7. The NMR tubes were pressurized (110 psi, i.e., approximately 8 bar total pressure) with p-H₂ bubbling through the solution at a flow of 90 scc/m to activate the catalyst and to ¹³C-hyperpolarize the sodium [1-¹³C]pyruvate solution. Activation of the catalyst took place for about 15 minutes at ambient temperature and magnetic field. For polarization build-up, the sample was placed in the magnetic field (typically about 0.4 μT) and a water bath at 5° C. to regulate the reaction temperature. The spectrum was acquired immediately following manual sample transfer to a 1.8 T benchtop NMR after 5 seconds, and the results are set forth in the top spectrum of FIG. 16. In addition, the bottom spectrum of FIG. 16 shows a single-scan thermally polarized ¹³C signal from 4 M sodium [1-¹³C]acetate using similar acquisition parameters. As is apparent from the results set forth in FIG. 16, the signal enhancement is ε˜16900 and polarization is about P(¹³C)˜2.17%.

### Example 18

This example illustrates a method of synthesis of N-(2,6-dimethyl-4-(3,3,4,4,5,5,6,6,7,7,8,8,8-tridecafluorooct-1-en-1-yl)phenyl)-2,2,2-trifluoroacetamide:

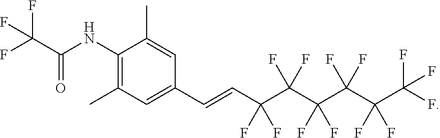 [SMILES: Cc1cc(/C=C/C(F)(F)C(F)(F)C(F)(F)C(F)(F)C(F)(F)C(F)(F)F)cc(C)c1NC(=O)C(F)(F)F]

In a glove box, palladium(II) acetate (0.16 g, 0.051 equiv, 0.71 mmol), sodium acetate (1.72 g, 1.50 equiv, 21.0 mmol), tricyclohexylphosphane (660 mg, 0.168 equiv, 2.35 mmol), tetrabutylammonium bromide (30 mg, 0.0067 equiv, 93 μmol) and 2,2,2-trifluoro-N-(4-iodo-2,6-dimethylphenyl)acetamide (4 g, 0.8 equiv, 0.01 mol) were added to a Schlenk flask with 10 mL of dimethyl formamide. The Schlenk flask was warmed to 90° C., then 3,3,4,4,5,5,6,6,7,7,8,8,8-tridecafluorooct-1-ene (4.84 g, 1 equiv, 14.0 mmol) was added to the reaction mixture and further heated to 120° C. The solution was stirred for 19 hours. After cooling, the mixture was filtered through Celite and washed with diethyl ether (50 mL). Water (50 mL) and diethyl ether (30 mL) were added, the organic phase was separated, and aqueous phase was extracted with Et₂O (3×15 mL). The combined organic layers were dried over anhydrous MgSO₄ and concentrated under reduced pressure to afford the crude product. Purification by column chromatography (hexane/DCM 4:1) gave fluoroamide N-(2,6-dimethyl-4-(3,3,4,4,5,5,6,6,7,7,8,8,8-tridecafluorooct-1-en-1-yl)phenyl)-2,2,2-trifluoroacetamide, which was further crystallized (5 g, 70%, white needles).

### Example 19

This example illustrates a synthesis of 2,6-dimethyl-4-(3,3,4,4,5,5,6,6,7,7,8,8,8-tridecafluorooctyl)aniline:

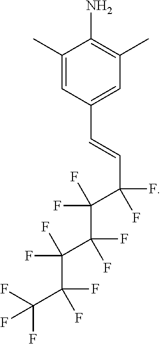 [SMILES: Cc1cc(/C=C/C(F)(F)C(F)(F)C(F)(F)C(F)(F)C(F)(F)C(F)(F)F)cc(C)c1N]

To a solution of N-(2,6-dimethyl-4-(3,3,4,4,5,5,6,6,7,7,8,8,8-tridecafluorooct-1-en-1-yl)phenyl)-2,2,2-trifluoroacetamide (0.14 g, 250 μmol) in n-butanol (1.5 mL), sodium hydroxide (0.10 g, 250 mmol) was added and the reaction mixture was heated to 110° C. for 21 hours. After cooling to room temperature, water (4 mL) and ethyl acetate (4 mL) were added and the organic phase was separated, washed with 1M solution of HCl (4 mL), saturated solution of NaHCO₃ (4 mL), and brine (4 mL). The aqueous phase was neutralized and extracted with ethyl acetate (3 times×4 mL). The organic layers were combined and dried over anhydrous magnesium sulfate and concentrated under reduced pressure to afford 2,6-dimethyl-4-(3,3,4,4,5,5,6,6,7,7,8,8,8-tridecafluorooctyl)aniline (0.50 g, 92%, brown crystals).

### Example 20

This example illustrates a synthesis of N,N′-Bis[2,6-dimethyl-4-(3,3,4,4,5,5,6,6,7,7,8,8,8-tridecafluorooct-t-en-t-yl)phenyl]ethane-1,2-diimine:

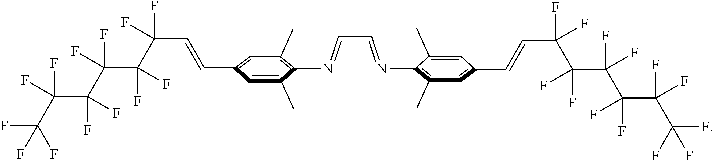

To the solution of 2,6-dimethyl-4-(3,3,4,4,5,5,6,6,7,7,8,8,8-tridecafluorooct-1-en-1-yl)aniline (0.5 g, 1 mmol) in ethanol (5 mL), 40% aqueous solution of glyoxal (0.1 mL, 1 mmol) and catalytic amount of formic acid (few drops) were added. The reaction mixture was stirred for 20 hours at room temperature, during which yellow precipitate was formed. The solid was filtered and washed with cold ethanol (3×5 mL) to give clean N,N-bis(2,6-dimethyl-4-(3,3,4,4,5,5,6,6,7,7,8,8,8-tridecafluorooctyl)phenyl)ethane-1,2-diimine (0.2 g, 20%, yellow powder).

### Example 21

This example illustrates a synthesis of 1,3-bis(2,6-dimethyl-4-(3,3,4,4,5,5,6,6,7,7,8,8,8-tridecafluorooct-1-en-1-yl)-1H-imidazol-3-ium-2-ide, chloride salt:

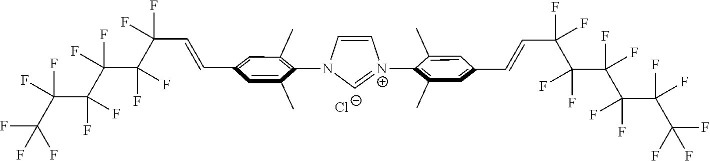

A flask was charged with N,N-bis(2,6-dimethyl-4-(3,3,4,4,5,5,6,6,7,7,8,8,8-tridecafluorooct-1-en-1-yl)phenyl)ethane-1,2-diimine (2.40 g, 2.08 mmol) and tetrahydrofuran (80 mL). The mixture was cooled to 0° C. and suspension of paraformaldehyde (262 mg, 2.91 mmol) and conc. HCl (112 mg, 3.12 mmol) in dioxane (0.78 mL) was slowly added. The mixture was heated to reflux overnight. Purification was carried out by column chromatography. ¹H NMR (400 MHz, CDCl₃) δ 2.27, 6.71, 7.32, 7.36, 7.67, 8.16, 9.59 ppm. ¹⁹F NMR (400 MHz, CDCl₃) δ−82.43, −112.52, −122.59, −123.89, −124.17, −127.33 ppm. MS (ESI) m/z (%): 964.8 [M+H]⁺ (100).

### Example 22

This example illustrates a synthesis of a fluorinated SABRE catalyst in accordance with an aspect of the invention.

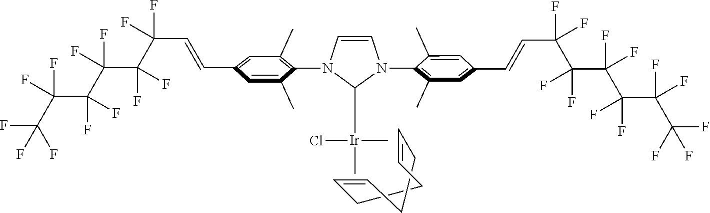

Potassium tert-butoxide (2.5 eq.) was added to a stirred solution of 1,3-bis(2,6-dimethyl-4-(3,3,4,4,5,5,6,6,7,7,8,8,8-tridecafluorooct-1-en-1-yl)phenyl)-1H-imidazol-3-ium-2-ide, chloride (2.2 equiv) in tetrahydrofuran at room temperature in a glove box. The resulting suspension was stirred for 30 min. A solution of [Ir(COD)Cl]₂ (1.0 eq.) was added and the resulting solution was stirred at room temperature for 2 hours. The solvent was removed under reduced pressure to give the crude product and dried overnight under vacuum. This sample was purified with flash chromatography DCM/hexane (4:1) to obtain the fluorinated SABRE catalyst. MS (ESI) m/z (%): 1266 [M-Cl]⁺ (100).

### Example 23

This example illustrates the hyperpolarization of sodium pyruvate using the SABRE catalyst in accordance with an aspect of the invention, as shown in Scheme 4.

Scheme 4. Hyperpolarization of [1-¹³C]Pyruvate Using the Shown Perfluorinated SABRE Catalyst with Dimethyl Sulfoxide as Co-Ligand.

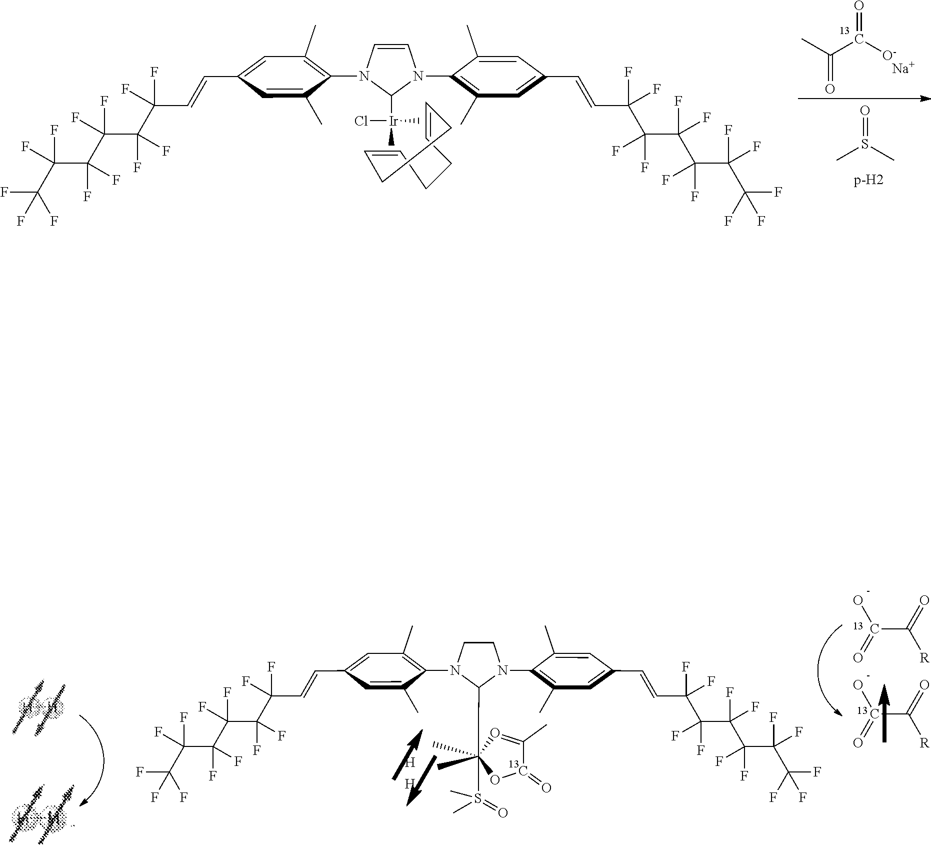

In the reaction scheme above, the efficient hyperpolarization transfer from p-H₂-derived hydrides to the ¹³C nuclear spin of [1-¹³C] pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate) and p-H₂ in deuterated methanol.

The SABRE samples were prepared in CD₃OD, using 20 mM sodium [1-¹³C]pyruvate, 7.6 mM perfluorinated SABRE catalyst shown in Scheme 4, and 50 mM dimethyl sulfoxide (DMSO), as described in Examples 6 and 7. The SABRE samples were exposed to the SABRE-SHEATH hyperpolarization conditions with the same set-up and optimum conditions described in Examples 6 and 7. The NMR tubes were pressurized (110 psi, i.e., approximately 8 bar total pressure) with p-H₂ bubbling through the solution at a flow of 90 scc/m to activate the catalyst and to ¹³C-hyperpolarize the sodium [1-¹³C]pyruvate solution. Activation of the catalyst took place for about 15 minutes at ambient temperature and magnetic field. For polarization build-up, the sample was placed in the magnetic field (typically about 0.4 μT) and a water bath at 5° C. to regulate the reaction temperature. The spectrum was acquired immediately following manual sample transfer to a 1.8 T benchtop NMR after 5 seconds, and the results are set forth in the top spectrum of FIG. 17. In addition, the bottom spectrum of FIG. 17 shows a single-scan thermally polarized ¹³C signal from 4 M sodium [1-¹³C]acetate using similar acquisition parameters. As is apparent from the results set forth in FIG. 17, the signal enhancement is ε˜19000 and polarization is about P(¹³C)˜4.91%.

### Example 24

This example illustrates a method of hyperpolarizing a [1-¹³C]pyruvate in accordance with an aspect of the invention, using a fluorous mixture instead of only deuterated methanol.

The hyperpolarization procedure of Example 6 was repeated using a mixture of nonafluorobutyl methyl ether (NFBME) and deuterated methanol instead of only deuterated methanol.

The fluorinated SABRE catalyst activation took less than 25 minutes, with the 13C polarization percentage shown in FIG. 18, and is performed by bubbling ˜95% p-H₂ at a flow rate of 90 standard cubic centimeters per minute (scc/m) at 8 atm p-H₂ partial pressure. The NMR samples contained about 25 mM sodium [1-¹³C]pyruvate, 7.4 mM perfluorinated SABRE catalyst of Example 4, and 40 mM dimethyl sulfoxide (DMSO) in 0.3 mL NFBME and 0.2 mL MeOD with the mixing field at 0.4 μT and a temperature of 0° C.

### Example 25

This example demonstrates the effects on hyperpolarization of [1-¹³C]pyruvate in a fluorous mixture, exhibited by changes in parahydrogen pressure and flow rate, as well as the effect of magnetic transfer field. In addition, the relaxation dynamics of the [1-¹³C]pyruvate were also studied.

Hyperpolarization of [1-¹³C]pyruvate was repeated using SABRE in SHield Enables Alignment Transfer to Heteronuclei (SABRE-SHEATH), as described in Example 24 above, and the effects of parahydrogen flow rate, as well as the effect of magnetic transfer field, were studied.

p-H₂ flow rate was evaluated and the polarization percentage results are set forth in FIG. 19. The NMR samples contained about 22 mM sodium [1-¹³C]pyruvate, 7.4 mM perfluorinated SABRE catalyst of Example 4, and 45 mM dimethyl sulfoxide (DMSO) in 0.3 mL NFBME and 0.2 mL MeOD with the mixing field at 0.4 T and a temperature of 0° C. As is apparent from the results set forth in FIG. 19, as the parahydrogen flow rate increased, the polarization percentage also increased.

The magnetic field in the micro Tesla regime was evaluated and the ¹³C polarization transfer magnetic field at 0° C. is set forth in FIG. 20. The NMR samples contained 22 mM sodium [1-¹³C]pyruvate, 7.4 mM perfluorinated SABRE catalyst of Example 4, and 46 mM dimethyl sulfoxide (DMSO) in 0.2 mL NFBME and 0.2 mL MeOD. The p-H₂ flow rate and pressure were 50 ssc/m and 110 PSI, respectively. As is apparent from the results set forth in FIG. 20, the best polarization transfer occurs at a mixing field between 0.3 μT and 0.5 μT. The optimum mixing field is 0.4 μT.

As is apparent from the results set forth in FIGS. 21A and 21B, the relaxation dynamics of [1-¹³C]-pyruvate show that the total P_{13C} (bound+free) build-up time (T_{b}=3.0±0.8 s) is substantially shorter than the corresponding T₁ value of 11.3±1.3 s, which allows to reach P_{13C} levels up to 6.02%. In addition, relaxation dynamics at earth field and 1.8 T are about the same as a non-fluorinated SABRE catalyst T₁=9.0±1.9 s and 16.0±1.4 s, respectively.

The simultaneous exchange of p-H₂ and [1-¹³C]pyruvate on activated Ir(F-IMes) catalyst leads to buildup of ¹³C hyperpolarization. In that respect, the top spectrum of FIG. 22 shows a representative spectrum of ¹³C-hyperpolarized [1-¹³C]-pyruvate with signal enhancement F of ˜38600 fold, corresponding to P_{13C} of ˜6.02% obtained via comparison of the NMR signal intensity to a reference sample (i.e., the bottom spectrum of FIG. 22, which shows a single-scan thermally polarized ¹³C signal from 4 M sodium [1-¹³C] acetate using similar acquisition parameters). The NMR samples contained 23 mM sodium [1-¹³C]pyruvate, 7.4 mM perfluorinated SABRE catalyst of Example 4, and 46 mM dimethyl sulfoxide (DMSO) in 0.2 mL NFBME and 0.2 mL MeOD. The p-H₂ flow rate and pressure were 90 ssc/m and 110 PSI, respectively, with the mixing field at 0.4 μT and a temperature of 0° C.

Temperature has a profound effect on the exchange rates of [1-¹³C]pyruvate on Complex 3b of FIG. 7. In recent work, Adelabu et al. (ChemPhysChem, 23, e202100839 (2022)) showed that the monotonic disappearance of free HP resonance at low temperatures happened due to the slow exchange rate of Complex 3b into the free state. At room temperature (e.g., 22° C.), the exchange of [1-¹³C]pyruvate with the polarization transfer complex was faster, leading to hyperpolarization of both free and bound 3b species in the expected pyruvate:pre-catalyst ratio. In order to rapidly release the HP pyruvate from 3b, the HP solution was rapidly warmed up then the sample was inserted in the NMR detector (TomHon et al., J. Am. Chem. Soc., 144(1) 282-287 (2022)). FIG. 23 shows a variable temperature SABRE-SHEATH experiment using the saturated perfluorinated SABRE catalyst of Example 4. The NMR samples (in nonafluorobutyl methyl ether (NFBME) and deuterated methanol) contained in 23 mM sodium [1-¹³C]pyruvate, 7.4 mM perfluorinated SABRE catalyst of Example 4, and 46 mM dimethyl sulfoxide (DMSO), wherein the mixing field was at 0.4 μT, and the parahydrogen pressure and flow rate were set at 110 PSI and 90 scc/m, respectively.

As demonstrated by FIG. 23, the exchange rate of Complex 3b into the free state is faster even at low temperature, such as −10° C., where most of the HP [1-¹³C]pyruvate is in a free state. By decreasing the temperature of ¹³C SABRE-SHEATH to 0° C., P_{13C} maximum is achieved. The exchange remains fast enough to build-up preferentially the “free” HP [1-¹³C]pyruvate over Complex 3b.

### Example 26

This example illustrates an exemplary method for isolating hyperpolarized sodium [1-¹³C]pyruvate, which includes biphasic extraction with an aqueous phase and a fluorinated phase.

The hyperpolarization procedure of Example 6 was repeated using a mixture of nonafluorobutyl methyl ether (NFBME) and deuterated methanol instead of only deuterated methanol. The SABRE sample was prepared with 23 mM sodium [1-¹³C]pyruvate, 7.4 mM perfluorinated SABRE catalyst of Example 4, and 46 mM dimethyl sulfoxide (DMSO) in 0.2 mL NFBME and 0.2 mL MeOD, wherein the mixing field was at 0.4 μT, and the parahydrogen pressure and flow rate were set at 110 PSI and 90 sec/m, respectively.

After the hyperpolarization procedure was completed, the sample was rapidly removed from the 0.40 μT field and transferred inside the NMR spectrometer at 1.8 T at room temperature. The NMR sample was depressurized and 400 μL of D₂O was added to the solution in order to drive the hyperpolarized sodium [1-¹³C]pyruvate into the aqueous phase. The pyruvate signal in the aqueous phase was collected, but the separation led to the formation of an emulsion, indicating that both bound and free pyruvate are present. See FIG. 24. In that respect, the top spectrum of FIG. 24 shows a representative spectrum of ¹³C-hyperpolarized [1-¹³C]-pyruvate with signal enhancement F of ˜10800 fold, corresponding to P_{13C} of ˜1.68% obtained via comparison of the NMR signal intensity to a reference sample (i.e., the bottom spectrum of FIG. 24, which shows a single-scan thermally polarized ¹³C signal from 4 M sodium [1-¹³C]acetate using similar acquisition parameters).

The aqueous phase containing the sodium [1-¹³C]pyruvate was evacuated and tested for iridium content and pyruvate concentration. The ICP-MS study showed a content of 637 ppb of iridium and the LCMS showed about 50-75% of the sodium [1-¹³C]pyruvate concentration was collected after filtration.

The fluorous mixture containing the perfluorinated SABRE catalyst in NFBME and MeOD was re-used for further sodium [1-¹³C]pyruvate hyperpolarization. After evacuation of the water phase, a solution containing 23 mM sodium [1-¹³C]pyruvate and 47 mM dimethyl sulfoxide in 0.2 mL CD₃OD was added to the fluorous mixture containing the perfluorinated SABRE catalyst from 4 days earlier. The hyperpolarization of sodium [1-¹³C]pyruvate was repeated, and showed a polarization of the [1-¹³C]pyruvate with a signal enhancement ε of ˜3090 fold, corresponding to P_{13C} of ˜0.48%, which was obtained via comparison of the NMR signal intensity to a reference sample, as shown in FIG. 25. The polarization of [1-¹³C]pyruvate is repeatable and showed about the same polarization level, demonstrating that the perfluorinated SABRE catalyst remains active for at least 4 days after initial use.

### Example 27

This example illustrates a synthesis of 1,3-dimesityl-4,5-bis(2-(perfluorophenyl)propyl)-4,5-dihydro-1H-imidazol-3-ium, triflate salt:

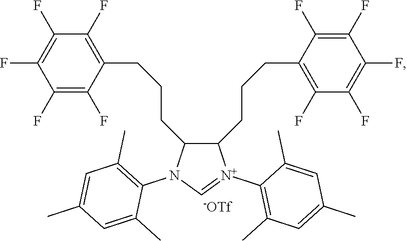 [SMILES: Cc1cc(C)c(N2C=[N+](c3c(C)cc(C)cc3C)C(CCCc3c(F)c(F)c(F)c(F)c3F)C2CCCc2c(F)c(F)c(F)c(F)c2F)c(C)c1 ; "-OTf"]

which was prepared in accordance with the synthesis sequence set forth in FIG. 26.

4-Pentafluorophenylbutanal (3). To a solution of the alcohol (2) (4.8 g, 20.0 mmol) in DCM (40 mL), was added Dess-Martin periodinane (16.96 g, 40.0 mmol) portionwise at 0° C. while being stirred under argon (5 minutes). The reaction was continued until the starting alcohol was consumed (2 hours). The resulting solution was concentrated to about 10.0 mL and adsorbed onto 10.0 g of silica and dried to a free flowing powder. The silica with the crude product was applied to a silica column (120.0 g), and elution with 5% ethyl acetate in hexanes yielded the product as colorless oil. Yield: 3.33 g (70%). ¹H NMR (CDCl₃): ¹H NMR (400 MHz, CDCl₃) δ 9.71 (t, J=1.3 Hz, 1H), 2.69 (tt, J=7.9, 1.8 Hz, 2H), 2.44 (td, J=7.3, 1.2 Hz, 2H), 1.86 (p, J=7.3 Hz, 2H). ¹³C NMR (101 MHz, CDCl₃) δ 200.89, 42.81, 21.49.

1,2,3,4,5-Pentafluoro-6-(4-iodobutyl)benzene (4). An ice-cooled solution of the alcohol (2) (16.0 g, 66.67 mmol) was stirred with imidazole (5.89 g, 86.67 mmol) and triphenylphosphine (20.96 g, 80.0 mmol) in DCM (100 mL) under argon. Iodine (20.32 g, 80.0 mmol) was added portionwise over a period of 15 minutes at 0° C. while being vigorously stirred until a slight yellow color persisted. The reaction mixture was diluted with hexanes (300 mL) and filtered. The filtrate was washed with saturated sodium thiosulfate (3×50 mL), water (3×100 mL), and dried with sodium sulfate. The clear solution was filtered and concentrated to an oil that was chromatographed over silica gel (220 g). Elution with hexanes yielded compound (4) as colorless oil. Yield: 21.0 g (90%). ¹H NMR (400 MHz, cdcl₃) δ 3.20 (t, J=6.8 Hz, 1H), 2.73 (tt, J=7.5, 1.8 Hz, 1H), 1.86 (dq, J=8.6, 6.8 Hz, 1H), 1.76-1.66 (m, 1H). ¹³C NMR (101 MHz, cdcl₃) δ 37.84, 30.01, 21.27, 5.39.

Iodo-(4-(pentafluorophenyl)butyl)triphenyl-λ⁵-phosphane (5). A solution of the iodobutane (4) (21.0 g, 60.0 mmol) and triphenylphosphine (17.29 g, 66.0 mmol) was refluxed under argon for 24 hours. The precipitated solid was filtered and washed with anhydrous ether and dried under high vacuum for 20 hours. Yield: 33.78 g (92%). ¹H NMR (400 MHz, CDCl₃) δ 7.73 (m, 15H), 3.92, 3.86 (m, 2H), 2.76, 2.74 (m, 2H), 2.07, 2.025 (m, 2H), 1.6 (m, 2H). ¹³C NMR (101 MHz, CDCl₃) δ 135.23, 135.20, 133.75, 133.65, 130.65, 130.53, 118.32, 117.46, 29.43, 29.27, 23.05, 22.55, 21.78, 21.74, 21.66.

Compound 6 To a solution of the phosphonium salt (5) (6.79 g, 11 mmol) in anhydrous THF (40 mL) was added potassium tert-butoxide in THF (2M, 12.5 mL, 25.0 mmol) dropwise and stirred under argon for 30 minutes. Aldehyde (3) (2.2 g, 9.24 mmol) in THF (5 mL) was added dropwise at room temperature and stirred for 3 hours. The solution was concentrated, and purified on flash silica (120 g). Elution with hexanes yielded the product as a colorless oil as mixture of cis/trans isomers (90:10). Yield: 3.2 g (72%). ¹H NMR (400 MHz, CDCl₃) δ 5.41 (m, 2H), 2.72, 2.68 (m, 4H), 2.12, 2.10, 2.06 (m, 4H), 1.64 (m, 4H). ¹³C NMR (101 MHz, CDCl₃) δ 129.46, 29.12, 26.76, 21.94

Compound 7. Olefin (6) (3.6 g, 8.1 mmol) in acetone (20 mL) and water (0.5 mL) was cooled to 0° C., and dibromamine-T (2.93 g, 8.91 mmol, Org. Biomol. Chem., 8, 1424-1430 (2010)) was added with vigorous stirring. After the addition, the solution was brought to room temperature and stirred until the starting material disappeared (30 minutes). The solution was quenched with solid sodium thiosulfate (2.0 g) and stirred until the color of bromine was completely discharged. The mixture was concentrated under reduced pressure, and the residue was diluted with water (50 mL) and extracted with EtOAc (3×30 mL). The combined organic layers were dried (MgSO₄), filtered, and concentrated under reduced pressure. The residue was chromatographed over flash silica (80 g) and eluted with 10% EtOAc in hexanes to yield the bromohydrin (7) as mixture of diastereomers and as colorless oil. Yield: 2.41 g (55%). ¹H NMR (400 MHz), CDCl₃) δ 4.02 (m, 1H), 3.48 (m, 1H), 2.73 (4H, CH₂—C₆F₅), 1.6-2.0 (m, 9H, —CH₂— and OH)). ¹³C NMR (101 MHz, CDCl₃) δ 143.76, 138.35, 129.62, 126.82, 59.74, 57.12, 35.29, 34.50, 27.39, 25.48, 21.74, 21.34.

Compound 8. The bromohydrin (7) (2.2 g, 4.06 mmol) was dissolved in DCM (10.0 mL) and stirred with 5.0 g of molecular sieves. Dess-Martin reagent (3.44 g, 8.12 mmol) was added and stirred until the starting material was completely consumed (30 minutes). The whole mixture was adsorbed onto flash silica (20.0 g) and dried to a free flowing powder. The silica with the crude product was loaded onto a flash silica (80.0 g) column and eluted with 0 to 20% EtOAc in hexanes over 20 minutes (80.0 mL/min; elution rate). Bromoketone (8) was eluted at 5% EtOAc in hexanes as a colorless syrup. Yield: 1.82 g (83%). ¹H NMR (400 MHz, CDCl₃) δ 4.25 (dd, J=8.3, 6.0 Hz, 1H), 2.94-2.81 (m, 1H), 2.80-2.68 (m, 4H), 2.67-2.52 (m, 1H), 2.09-1.86 (m, 4H), 1.86-1.59 (m, 2H). ¹³C NMR (101 MHz, CDCl₃) δ 202.53, 52.19, 38.18, 32.31, 26.87, 23.21, 21.62, 21.45.

Compound 9. Sodium bicarbonate (0.56 g, 6.68 mmol) was added to a stirred solution of bromoketone (8) (1.8 g, 3.34 mmol) in anhydrous acetonitrile (10.0 mL). Trimethylaniline formamidine (0.94 g, 3.34 mmol) was then added to the stirred solution as a solid, and the reaction mixture was heated to 60° C. with the exclusion of moisture. After the consumption of the bromoketone (70 hours), the reaction mixture was filtered, concentrated under reduced pressure, and chromatographed over flash silica (80 g). Elution with 20% DCM in hexanes yielded the ketoamidine (10) as a colorless syrup. Yield: 1.2 g (49%). ¹H NMR δ (CDCl₃) 6.91 9s, 1H), 6.81 (s, 1H), 6.71 (s, 1H), 6.68 (s, 2H), 4.53 (m, 1H), 3.08 (m, 1H), 2.57 (m, 2h), 2.43 (m, 2H), 2.35 (m, 2H), 2.36 (s, 3h), 2.2 (s, 3H), 2.13 (s, 3H), 2.01 (s, 3H), 1.92 (s, 6H), 1.8 (m, 2H). ¹³C NMR (101 MHz, CDCl₃) δ 208.19, 151.90, 138.19, 131.45, 129.47, 128.74, 64.33, 60.38, 41.49, 28.39, 25.50, 22.96, 20.56, 18.44, 18.41, 18.23, 14.18. MS: 739.1 [M+H].

Compound 11 (triflate salt). To an ice-cooled solution of the keto amidine (0.994 g, 1.35 mmol) in ethanol, lithium borohydride (2M in THF, 0.675 ml, 1.35 mmol) was added and stirred under argon for 24 h at 0-5° C. The reaction mixture was poured into 50 ml of water and extracted with ethyl acetate (3×50 ml). The combined organic layers were dried (sodium sulfate), and filtered, and concentrated to a paste at room temperature. The above paste was dissolved in 5.0 ml of anhydrous benzene and cooled to 0° C. DIPEA (0.523 g, 4.05 mmol) was added and stirred under argon. Trifluoromethanesulfonic anhydride (1.104 g, 1.34 mmol) was then added dropwise and stirring was continued for 1 h. LC/MS indicated the complete consumption of peak corresponding to 741. The mixture was adsorbed onto 10.0 g of silica gel and dried to a free flowing powder. The silica with the crude product was loaded onto an empty loading column placed on the top of a 80.0 g silica column and chromatographed. Elution with 4% MeOH in DCM yielded the product as a mixture of cis/trans isomers and as brown paste. Yield: 0.338 g (33%). ¹H NMR (CDCl₃) δ 8.82 (s, 1H), 6.93 (s, 4H), 4.15 (m, 2H), 2.61 (m, 4H), 2.25, 2.23, 2.21 (3S, 18H), 1.77 (m, 4H), 1.41 (m, 4H). ¹³C NMR (101 MHz, CDCl₃) δ 159.41, 141.00, 135.66, 134.43, 130.60, 129.96, 128.58, 68.76, 66.17, 32.48, 26.91, 24.65, 21.71, 20.97, 18.38, 18.11. MS: 723.0 [M+H].

All references, including publications, patent applications, and patents, cited herein are hereby incorporated by reference to the same extent as if each reference were individually and specifically indicated to be incorporated by reference and were set forth in its entirely herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An MRI probe infusion device comprising:
(i) one or more reaction chambers, each reaction chamber comprising:
(a) a structure configured to attenuate a magnetic field within the reaction chamber from an external source, wherein a strength of the magnetic field within the reaction chamber from the external source is less than a threshold value;
(b) one or more inlet ports;
(c) a coil configured to generate an electro-magnetic field within the reaction chamber;
(d) one or more temperature control devices; and
(e) one or more outlet ports;
(ii) one or more MRI probe separators configured to receive a reaction mixture containing a perfluorinated SABRE catalyst, a solvent, and a hyperpolarized MRI probe from the one or more reaction chambers and extract the hyperpolarized MRI probe from the reaction mixture; and
(iii) one or more MRI probe collectors configured to form a solution containing a desired concentration of the hyperpolarized MRI probe.

2. The MRI probe infusion device of claim 1, wherein the structure is a mu-metal shield that attenuates a magnetic field from the external source to have a strength of less than or equal to 10 nT in the one or more reaction chambers.

3. The MRI probe infusion device of claim 1, wherein the one or more inlet ports include one or more gas ports and one or more liquid ports.

4. The MRI probe infusion of device of claim 1, wherein each reaction chamber is configured to withstand a gas pressure of at least 10 bars.

5. The MRI probe infusion device of claim 1, wherein the magnetic field within the reaction chamber induced by the coil is between 0-200 milliTeslas.

6. The MRI probe infusion device of claim 1, wherein the one or more temperature control devices comprise a non-magnetic heating element and/or cooling element configured to maintain a temperature of the reaction chamber between −25° C. to 100° C.

7. The MRI probe infusion device of claim 1, wherein the one or more reaction chambers are equipped to perform hyperpolarization with a reaction mixture containing the perfluorinated SABRE catalyst, the solvent, and a substrate to be hyperpolarized into the hyperpolarized MRI probe.

8. The MRI probe infusion device of claim 7, wherein the solvent is a one phase system or a two phase system comprising water, methanol, ethanol, a fluorous solvent, or a mixture thereof.

9. The MRI probe infusion device of claim 1, wherein the one or more MRI probe separators are configured to separate the hyperpolarized MRI probe from the perfluorinated SABRE catalyst by one of:
filtration;
extraction; or
column chromatography.

10. The MRI probe infusion device of claim 1, further comprising a gas trap, a gas leak detector, and/or an oxygen level monitor.

11. The MRI probe infusion device of claim 1, further comprising a processor configured to execute instructions that cause the processor to:
control a flow of gas and/or liquid through the device,
monitor a safety metric of the device and/or environment,
administer a desired quantity of the hyperpolarized MRI probe to the patient, or
calculate a decay rate of the hyperpolarized MRI probe as a function of a rate of flow of the gas and/or the liquid.

12. The MRI probe infusion device of claim 1, wherein the one or more MRI probe collectors include one or more dryers.

13. The MRI probe infusion of device of claim 1, wherein the one or more reaction chambers include at least two reaction chambers configured to be operable in series or in parallel.

14. The MRI probe infusion device of claim 1, wherein components of the device are made of non-magnetic materials or plastics.

15. A method of administering a hyperpolarized MRI probe to a patient in need thereof, the method comprising:
(i) providing an MRI probe infusion device according to claim 1,
(ii) supplying to one or more of the reaction chambers a reaction mixture comprising a perfluorinated SABRE catalyst comprising a d-block element and a perfluorinated ligand, a solvent, a co-ligand, and a substrate to be hyperpolarized into an MRI probe,
(iii) agitating the reaction mixture, wherein the agitation is provided via bubbling parahydrogen gas or a mixture of parahydrogen and nitrogen gas through the reaction mixture,
(iv) applying a magnetic field suitable for hyperpolarization of the perfluorinated SABRE catalyst and the substrate to hyperpolarize the substrate into a hyperpolarized MRI probe,
(v) separating the hyperpolarized MRI probe from the reaction mixture by at least one of filtration, extraction, or column chromatography to obtain a solution containing the hyperpolarized MRI probe,
(vi) concentrating the hyperpolarized MRI probe present in the solution obtained in step (v) to obtain a concentrate and reconstituting the concentrate into a solution of desired concentration of the hyperpolarized MRI probe for administering to the patient;
(vii) analyzing at least one of a purity or a concentration of the hyperpolarized MRI probe present in the solution; and
(viii) administering the hyperpolarized MRI probe to the patient.

16. The method of claim 15, wherein the solvent is selected from a perfluorohexane/diethyl ether mixture, a methoxy nonafluorobutane and ethyl acetate mixture with a non-polar solvent, a perfluorohexane and ether mixture, a perfluorobutyl methyl ether and ethyl acetate mixture, an ether, a fluorocarbon derivative of THF FC 75, a decafluoromethoxy trifluoromethyl pentane, a hexafluoro propanol, a nonafluorobutyl methyl ether, a perfluoromethyl cyclohexane, a perfluoroalkane, a perfluorohexane, and a methoxy nonafluorobutane.

17. The method of claim 15, wherein the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

18. The method of claim 15, wherein the perfluorinated ligand is of Formula (I): [Lₘ-(NHC)—(Y—Z)_{q}] or a salt thereof, and wherein:
each L is independently selected from hydrogen, adamantyl, a substituted or unsubstituted aromatic, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4, and
q is an integer from 1 to 3.

19. The method of claim 18, wherein the perfluorinated tag is one of:
a perfluorinated C₃₋₆₀ group comprising only carbon and fluorine atoms;
a perfluorinated C₃₋₄₀ group comprising only carbon and fluorine atoms; or
a perfluorinated C₃₋₂₀ group.

20. The method of claim 15, wherein the perfluorinated ligand is selected from one of: 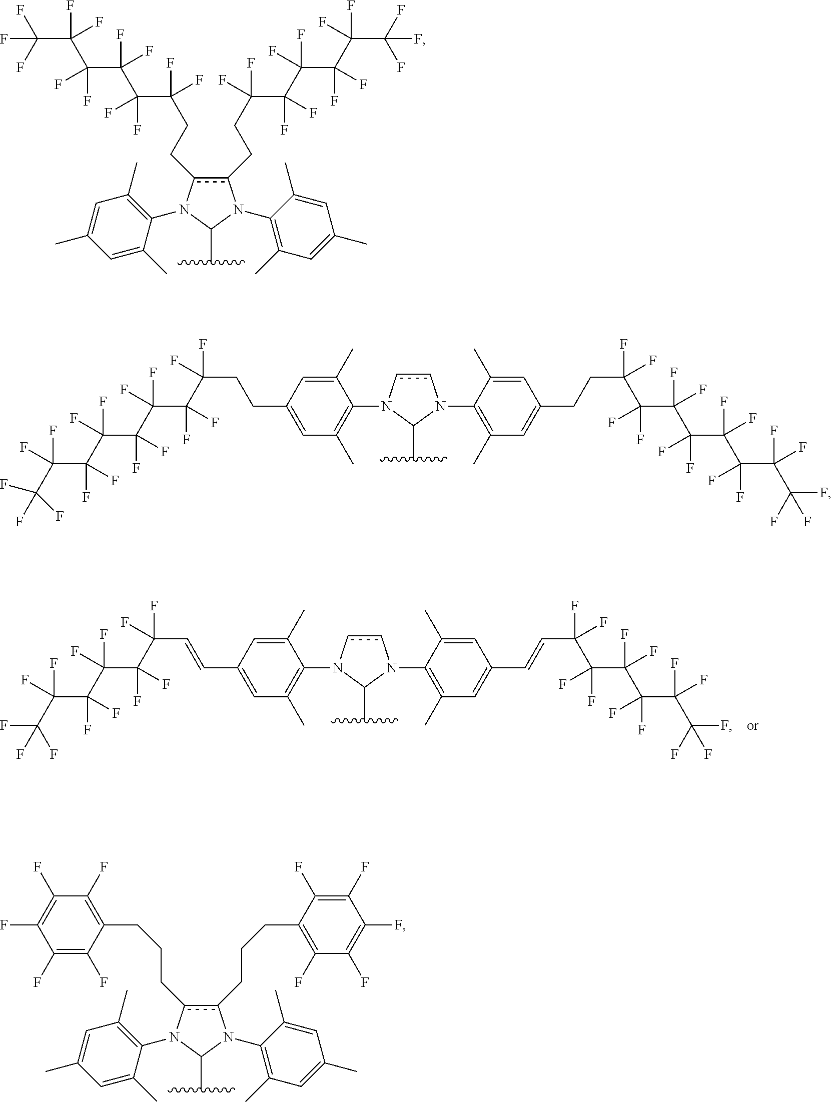
or a salt thereof, and wherein: [symbol]
is a single bond or a double bond, and [symbol]
represents the bond to the d-block element via the carbene.
